(19) United States
(12) Patent Application Publication
(10) Pub. No.: US 2014/0024683 A1
(43) Pub. Date: Jan. 23, 2014

(54) CHLORIDE CHANNEL AND CHLORIDE TRANSPORTER MODULATORS FOR THERAPY IN SMOOTH MUSCLE DISEASES

(71) Applicants: Charles William EMALA, Woodcliff Lake, NJ (US); Peter YIM, Fort Lee, NJ (US); George GALLOS, Tenafly, NJ (US); Donald LANDRY, New York, NY (US)

(72) Inventors: Charles William EMALA, Woodcliff Lake, NJ (US); Peter YIM, Fort Lee, NJ (US); George GALLOS, Tenafly, NJ (US); Donald LANDRY, New York, NY (US)

(21) Appl. No.: 14/035,463

(22) Filed: Sep. 24, 2013

Related U.S. Application Data
(63) Continuation-in-part of application No. PCT/US2012/030201, filed on Mar. 22, 2012.
(60) Provisional application No. 61/467,739, filed on Mar. 25, 2011.

(51) Int. Cl. A61K 31/44; A61K 45/06; A61K 31/196

(52) U.S. Cl. CPC A61K 31/44; A61K 31/196; A61K 45/06
USPC 514/352; 514/562

(57) ABSTRACT

The present invention provides, inter alia, methods and pharmaceutical compositions for preventing, treating, or ameliorating the effects of a disease characterized by altered smooth muscle contractility, such as e.g., asthma and chronic obstructive pulmonary disease.

### CROSS-REFERENCE TO RELATED APPLICATIONS

This application is a continuation-part of and claims benefit to International Application No. PCT/US2012/030201 filed Mar. 22, 2012, which claims priority to U.S. Provisional Patent Application No. 61/467,739, filed Mar. 25, 2011. The entire content of the above applications is hereby incorporated by reference as if recited in full herein.

### GOVERNMENT FUNDING

This invention was made with government support under GM065281 and GM008464 awarded by the National Institutes of Health. The government has certain rights in the invention.

### FIELD OF THE INVENTION

The present invention relates to, inter alia, pharmaceutical compositions, and methods for modulating calcium-activated chloride channel (CaCC) activity or both CaCC and sodium-potassium-chloride co-transporter (NKCC) activity.

### BACKGROUND OF THE INVENTION

The National Heart, Lung and Blood Institute of the National Institutes of Health estimate that over 22 million adults and 6 million children in the United States have asthma. The worldwide prevalence of asthma is between 7-10%, and it is implicated in 1/250 deaths (Masoli et al., 2004). It is the leading medical cause for school absenteeism in children and the world-wide incidence of asthma is increasing (Duan, 2009). The incidence of asthma continues to increase globally with an associated increase in the morbidity and mortality associated with this common disease (Duan, 2009). Despite this pandemic of asthma, the pharmacologic approach to asthma has changed little in several decades, relying primarily on inhaled β₂-adrenoceptor agonists and anti-inflammatory therapies. Although acutely effective, chronic therapy with long-acting β₂-agonists (LABA) is associated with an increased death rate from asthma (Kazani et al., 2010). The pathogenesis of asthma involves chronic airway inflammation, increased mucous secretion, irritability of airway parasympathetic nerves, and hypertrophy and hyperresponsiveness of airway smooth muscle. Relaxation of airway smooth muscle remains an important goal in the acute treatment of bronchospasm, although no new classes of drugs have been developed in the past few decades. Thus, there is an urgent need for alternative therapies due to patients who are refractory to this therapy and the increased mortality associated with the use of long-acting β₂-adrenoceptor agonists (Kazani et al., 2010).

Furthermore, treatment options for patients with bronchospasm and other hyperreactive airway are limited. For example, bronchospasm in association with intubation and airway suctioning are common clinical problems for anesthesiologists and intensivists. Perioperative bronchospasm refractory to first line agents such as β-agonists continues to challenge healthcare professionals. Bronchospasm comprises 2% of claims in the closed-claims database, but 90% of bronchospasm involved severe brain injury or deaths (Woods et al., 2009). Bronchospasm is precipitated by noxious stimuli such as instrumentation (tracheal intubation), inhaled irritants, drugs or perioperative complications (aspiration, infection, trauma). The incidence of intraoperative bronchospasm has been reported to be up to 20% in active asthmatics, and poorly controlled asthma is a major risk factor (Liccardi et al., 2012). β-adrenoceptor agonists have been in the mainstay of therapy for several decades with little progress in identifying improved novel therapies. There is a need for novel therapeutics to treat acute bronchospasm and control severe asthma as many patients are resistant to current therapies.

Asthma involves a complex interplay with many cell types including nerves, inflammatory cells, airway epithelium and airway smooth muscle (ASM). ASM is recognized as one important regulator of airway tone in asthma. Recent interest has re-focused on the role of ASM in asthma as airway remodeling, including increased ASM mass, which is now recognized as an important component of chronic asthma. Moreover, one of the more recent innovative therapies for asthma is directly related to the reduction of ASM mass from moderate sized airways through thermoablation techniques (Rubin, 2010). Although current anti-inflammatory, anti-cholinergic, and β₂-agonist therapies all target different phenotypic aspects of ASM function, no new approaches directed at ASM have been identified in many decades.

Although the requirement for calcium in smooth muscle contraction has been recognized for a long time, elevation of calcium alone does not dictate the magnitude of smooth muscle contraction: calcium sensitivity of contractile proteins is modulated by the phosphorylation state of contractile proteins and their regulatory kinases/phosphatases. This may explain the long recognized finding that bradykinin receptors greatly increase calcium in ASM but cause rather weak contractions and the very recent discovery of bitter taste receptors in ASM that actually increase calcium but induce smooth muscle relaxation (Deshpande et al., 2010). Emerging evidence in ASM research suggests that calcium entry following membrane depolarization activates RhoA (Liu et al., 2005), a classic upstream modulator of the phosphorylation state of myosin phosphatase and in turn myosin light chain, a final determinant of contractile sensitivity to calcium. There is even evidence in other cell types that a change in membrane potential alone may directly activate RhoA (independent of calcium entry), a fundamental paradigm shift in the understanding of ASM contraction.

Contractile agonists classically couple via Gq proteins to the synthesis of inositol phosphates and the release of calcium from intracellular sarcoplasmic reticulum stores, but the sensitivity of the contractile apparatus to this calcium (Liu et al., 2006) is another important level of contractile regulation. The importance of calcium sensitivity relative to cytosolic calcium levels is highlighted by the observation that elevation of intracellular calcium alone (e.g. bradykinin or bitter taste receptor activation) is insufficient to induce contraction. Traditionally the importance of extracellular calcium and membrane potential in ASM has been thought to be less important than in other smooth muscle subtypes (e.g. vascular smooth muscle). However, the recent identification of the role of voltage-sensitive transient receptor potential (TRP) channels which contribute to the refilling of intracellular calcium stores (White et al., 2006) and the demonstration that membrane potential can modulate calcium sensitivity via the small G protein rho A (Liu et al., 2005; Janssen et al., 2004), has reaffirmed an important role for both extracellular calcium and membrane potential in the control of ASM contraction.

Chloride channels are a potential novel target on airway smooth muscle. Chloride currents are important in airway smooth muscle contraction, as activation leads to chloride efflux from the cell, which leads to depolarization of the membrane that further triggers a voltage-dependent calcium influx and further depolarization and contraction (Janssen et al., 2006). Chloride channel modulators have shown promise as a potential novel class of therapeutics to relax airway smooth muscle (Townsend et al, 2012). Modulation of the ligand-gated GABA_{A} (Gallos et al., 2012) and glycine channel (Yim et al., 2011), calcium activated chloride channels (CaCCs) (Huang et al., 2012; Zhang et al., 2013), and the sodium potassium chloride cotransporter (NKCC) (Bianco et al., 1988; Iwamoto et al., 2001; Stevens et al., 1992) have all been shown to be important in airway smooth muscle relaxation. Chloride channels are ubiquitous, and blockers are currently being investigated as possible therapeutic targets in vascular smooth muscle for hypertension, kidney for diuretics, gastrointestinal smooth muscle for diarrhea and for possible use against tumors in cancer therapy (Verkman et al., 2009).

The efflux of chloride via calcium-activated chloride channels (CaCCs) is known to contribute to the depolarization of the plasma membrane following exposure of contractile agonists (Hirota et al., 2006; Janssen et al., 1995), such as acetylcholine and caffeine. Although the efflux of chloride through the plasma membrane has been identified as the major contributor to plasma membrane depolarization, the fundamental importance of ASM membrane potential in contractile tone has been questioned. Previous attempts to relax ASM via individual blockade of only these channels have shown limited effects. For example, unlike vascular smooth muscle, dihydropyridine therapies directed against voltage dependent L-type calcium channels, one of the earliest discovered voltage-dependent channels in ASM (Kotlikoff et al., 1992), were ineffective at treating asthma (Gupta et al., 1993, Talwar et al., 1993). It was recognized that the range of membrane potential required for activation of this channel was not commonly achieved during depolarization of ASM.

However, at least six findings continue to support a mechanistic importance for membrane potential in the control of ASM tone: (1) a component of β-adrenoceptoragonist relaxation of ASM involves potassium efflux through calcium activated potassium channels (K_{Ca}) inducing relative plasma membrane hyperpolarization; (2) refilling of sarcoplasmic reticulum with calcium following a classic contractile agonist exposure involves opening of voltage-sensitive plasma membrane calcium channels (stored operated calcium entry (SOCE)) likely including non-selective cation channels of the transient receptor potential (TRP) family of which both TRP—C (Corteling et al., 2004) and TRP-V (Jia et al., 2004) members are recently identified on ASM; (3) the recent discovery of the expression of both GABA (Gallos et al., 2008) and glycine-modulated (Yim et al., 2011) chloride channels on ASM that modulate contractile tone; (4) the recent discovery that bitter taste receptors relax ASM despite an increase in intracellular calcium through a hypothesized opening of K_{ca} (a membrane hyperpolarizing event) (Deshpande et al., 2010); (5) changes in membrane potential have been shown to activate both M3 muscarinic receptors (Gq-coupled) in ASM cells (Billups et al., 2006) and M2 muscarinic receptors (Gi-coupled) (Ben-Chaim et al., 2006) independent of receptor occupancy by ligand; and (6) the characterization of T-type calcium channels in ASM (Yamakage et al., 2001) which are activated within membrane potential ranges achieved during agonist-induced ASM contraction and depolarization (Janssen, 1997). Moreover, evidence that inositol phosphates can in turn regulate membrane potential (Zhang et al., 1993; Gromada, 1996) suggests that second messenger regulation of intracellular SR calcium release and plasma membrane potential do not operate in isolation.

In view of the foregoing, improved compositions and methods for modulating CaCC and NKCC activity in vivo are needed. The present invention is directed to meeting these, and other, needs.

### SUMMARY OF THE INVENTION

In the present invention, it has been found that depolarization of the plasma membrane and intracellular concentrations of chloride are decreased by simultaneous blockade of CaCCs and the NKCCs. This results in (1) impaired refilling of sarcoplasmic reticulum calcium stores due to inadequate intracellular chloride available to influx into the SR to balance charge generation (Janssen, 2002) during calcium refilling and (2) a decrease in membrane depolarizing-dependent activation of rhoA (Janssen et al., 2004), a key modulator of smooth muscle calcium sensitivity. The simultaneous blockade of CaCCs and the NKCCs interrupts the plasma membrane's ability to effectively cycle chloride in and out of the cell, leading to direct relaxation of human ASM. Additionally, in the present invention, it has been found that blockade of CaCC causes membrane hyperpolarization, and that the simultaneous blockade of NKCC shifts the equilibrium potential of chloride, thereby attenuating depolarization.

In the present invention, it has also been found that combining two pharmacologic therapies, originally evaluated separately, have profound effects on the ability of human ASM to constrict to acetylcholine, a classic endogenous or exogenous constrictor of ASM. Both of these pharmacologic tools target different aspects of chloride handling in ASM cells which then modulate both sarcoplasmic reticulum refilling of calcium as well as the sensitivity of the contractile apparatus to calcium: two fundamental cellular aspects of smooth muscle contraction.

An especially exciting aspect of these findings is that members of both drug classes (e.g., talnifumate as a pro-drug of niflumic acid (CaCC inhibitor) (Walker et al., 2006) used for anti-inflammatory properties and e.g., bumetanide and furosemide (NKCC inhibitors) as a diuretic, for example) have been previously used clinically for other therapies, encouraging rapid clinical translation of these studies. Thus, the present invention includes a novel therapeutic approach to the treatment of bronchospastic airway diseases that may circumvent the current limitations of β₂-adrenoceptor agonist therapy.

For example, one embodiment of the present invention is a method of treating or ameliorating the effects of a disease characterized by altered smooth muscle contractility. This method comprises administering to a patient suffering from such a disease an effective amount of a calcium-activated chloride channel (CaCC) modulator and a sodium-potassium-chloride co-transporter (NKCC) modulator.

Another embodiment of the present invention is a pharmaceutical composition for treating or ameliorating the effects of a disease characterized by altered smooth muscle contractility. This composition comprises a pharmaceutically acceptable carrier, a CaCC modulator, and a NKCC modulator.

Yet another embodiment of the present invention is a method of relaxing airway smooth muscle. This method comprises administering to a patient in need thereof an effective amount of a CaCC modulator and a NKCC modulator.

An additional embodiment of the present invention is a method of preventing the effects of a disease characterized by altered smooth muscle contractility. This method comprises administering to a patient suffering from such a disease an effective amount of a calcium-activated chloride channel (CaCC) inhibitor and a sodium-potassium-chloride co-transporter (NKCC) inhibitor.

Another embodiment of the present invention is a pharmaceutical composition for preventing the effects of a disease characterized by altered smooth muscle contractility. This composition comprises a pharmaceutically acceptable carrier, a CaCC inhibitor, and a NKCC inhibitor.

A further embodiment of the present invention is a method of blocking the onset of an airway contraction. This method comprises administering to a patient in need thereof an effective amount of a CaCC inhibitor and a NKCC inhibitor.

Another embodiment of the present invention is a pharmaceutical composition for treating or ameliorating the effects of a disease characterized by altered smooth muscle contractility. This pharmaceutical composition comprises a pharmaceutically acceptable carrier and a CaCC inhibitor selected from the group consisting of

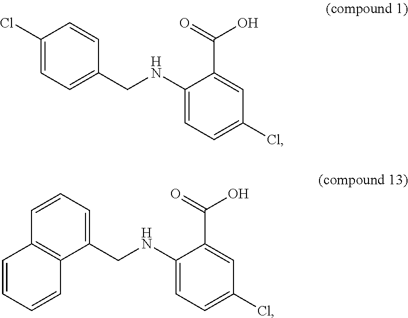 [SMILES: O=C(O)c1cc(Cl)ccc1NCc1ccc(Cl)cc1 ; O=C(O)c1cc(Cl)ccc1NCc1cccc2ccccc12]

a pharmaceutically acceptable salt thereof, and combinations thereof.

An additional embodiment of the present invention is a method for treating or ameliorating the effects of a disease characterized by altered smooth muscle contractility. This method comprises administering to a patient suffering from such a disease an effective amount of a CaCC inhibitor selected from the group consisting of

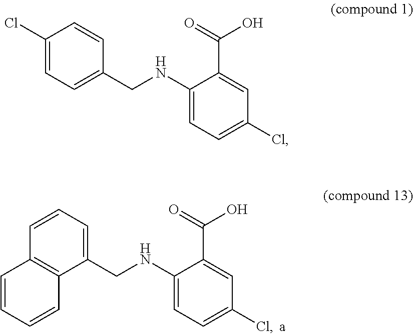 [SMILES: O=C(O)c1cc(Cl)ccc1NCc1ccc(Cl)cc1 ; O=C(O)c1cc(Cl)ccc1NCc1cccc2ccccc12]

pharmaceutically acceptable salt thereof, and combinations thereof.

A further embodiment of the present invention is a method of treating or ameliorating the effects of a disease characterized by altered smooth muscle contractility. This method comprises administering to a patient suffering from such a disease an effective amount of a sodium-potassium-chloride co-transporter (NKCC) inhibitor and a calcium-activated chloride channel (CaCC) inhibitor selected from the group consisting of

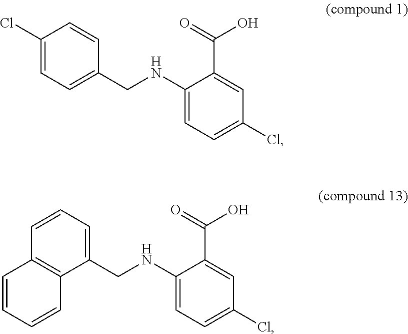 [SMILES: O=C(O)c1cc(Cl)ccc1NCc1ccc(Cl)cc1 ; O=C(O)c1cc(Cl)ccc1NCc1cccc2ccccc12]

a pharmaceutically acceptable salt thereof, and combinations thereof.

Another embodiment of the present invention is a method of treating or ameliorating the effects of a disease characterized by altered smooth muscle contractility. This method comprises administering to a patient suffering from such a disease an effective amount of a CaCC modulator, which is:

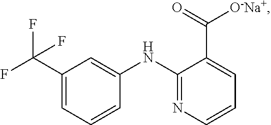

and a NKCC modulator.

An additional embodiment of the present invention is a pharmaceutical composition for treating or ameliorating the effects of a disease characterized by altered smooth muscle contractility. This composition comprises a pharmaceutically acceptable carrier, a CaCC modulator, which is

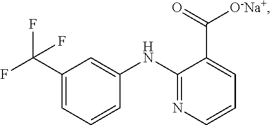

and a NKCC modulator.

### BRIEF DESCRIPTION OF THE DRAWINGS

FIG. 1 is a graph showing that simultaneous blockade of the CaCC and NKCC with 100 μM each of niflumic acid and bumetanide blocks repetitive acetylcholine (Ach)-induced contractions in human airway smooth muscle. Note the progressive decrease in the magnitude of the acetylcholine-induced contractions following 4 repetitive pretreatments with niflumic+bumetanide (top tracings) but not with other pretreatments. Progressive decrease with repetitive contractions is consistent with depletion of calcium from intracellular stores. Tracings from only pretreatments #2, #4 are shown for figure clarity. The tracings shown are representative of 8 airways from 4 patients.

FIG. 2. is a bar graph showing acetylcholine-induced contractions in human airway smooth muscle strips following pretreatment with 100 μM niflumic acid, 100 μM bumetanide, or both. Asterisk (*) indicates p<0.05. N=3-5.

FIG. 3A is a bar graph showing organ bath muscle force studies using guinea pig tracheal rings. Tetraethylammonium contractions were induced and muscle force was measured after treatments with 100 μM niflumic acid (CaCC blocker), 10 μM nifedipine, or 300 μM furosemide (NKCC blocker). N=4-5. Asterisk (*) indicates p<0.001 compared to basal. Double asterisks (**) indicate p<0.001 compared to basal. Triple asterisks (***) indicate p<0.001 compared to basal. FIG. 3B is a bar graph showing organ bath muscle force studies using guinea pig tracheal rings. Tetraethylammonium contraction (% acetycholine contraction control) comparing normal to decreased bath chloride concentration. Sodium gluconate was used to correct osmolarity. Muscle forces were proportional to the extracellular chloride concentration. N=4-6. Asterisk (*) indicates p<0.05. Double asterisks (**) indicate p<0.001 compared to 124 mM [Cl].

FIG. 4 shows in vivo airway pressure measurements in guinea pig. Representative pulmonary inflation in guinea pigs pretreated 12 minutes before administration of acetylcholine with either vehicle (100 μl DMSO, intravenous (i.v.)) or niflumic acid (5 mg, i.v.)+furosemide (5 mg, i.v.). Acetylcholine (14 μg/kg, i.v.) was then injected 6 times at 30 second intervals. Airway responses to injections 1, 3, and 5 are shown for clarity. Note the sequential decrease in the magnitude of airway constriction in response to acetylcholine following pretreatment with niflumic acid+furosemide (representative of 2 animals).

FIG. 5A-B show representative membrane potential measurements in human airway smooth muscle cells by (FIG. 5A) relative fluorescent unit (RFU) changes of FLIPR potentiometric probe or (FIG. 5B) eletrophysiologic recordings of voltage changes under current damp in whole cell configuration. Niflumic acid (100 μM) hyperpolarized the cell membrane while bumetanide (10 μM) was without effect. The measurements shown are representative of 4 independent recordings.

FIG. 6A-6B show representative tracings of intracellular chloride in human airway smooth muscle cells following blockade of CaCC (100 μM niflumic acid) (FIG. 6A) or blockade of NKCC (10 μM bumetanide) (FIG. 6B). Blockade of CaCC increases intracellular chloride quenching MQAE fluorescence while blockade of NKCC blocks chloride refilling unquenching MQAE fluorescence. Tracings shown are representative of 3 measurements in separate cell populations.

FIGS. 7A and 7B are bar graphs showing membrane potential (current clamp, whole cell) (FIG. 7A) and intracellular calcium (fluo4-AMfluorescence) (FIG. 7B) in human airway smooth muscle cells in response to 10 mM tetraethylammonium (TEA)-Cl or 60-75 mM potassium (K)-gluconate. TEA and K gluconate depolarized the cell, but only K gluconate increased calcium. Asterisk (*) indicates p<0.05 compared to control. N=3-6.

FIG. 8 is a bar graph showing RhoA activation in human airway smooth muscle cells. Primary cultures of cells were treated for 2 minutes with 10 mM tetraethylammonium (TEA)-acetate (Ac), or for various times with 60 mM potassium (K)-gluconate before cell solubilization and isolation of activated (GTP-bound) rhoA by rhotekin-binding pull down assay and detection by immunoblot.

FIG. 9 shows representative tracings of force measurements on human airway smooth muscle strips. Control contractions were performed with an EC₅₀ concentration of acetylcholine. Each strip was either treated with 100 μM niflumic acid and 10 μM bumex or 0.1% ethanol control and contracted with an EC₅₀ of acetylcholine. The strips were then thoroughly washed and recontracted.

FIG. 10 is a bar graph showing an analysis of force measurements on human airway smooth muscle strips. All measurements were normalized as percentages of the control EC₅₀ acetylcholine contraction. Control vs. the bumex/niflumic treatment group shows significant blockade of a control EC₅₀ acetylcholine contraction. Asterisk (*) indicates p<0.05. N=3.

FIG. 11A shows a representative intracellular whole cell tracing of a single guinea pig airway smooth muscle cell, treated with 10 mM TEA (K channel blocker). The cell was voltage clamped in a step protocol, with voltages ranging from about 40 to 100 mV recorded at intervals of 10 mV. The equilibrium potential was recoded at 2.2 mV. The recoding is linear around 0 mV with an exponential rise in the positive mV range consistent with a Cl current. FIG. 11B shows whole cell intracellular voltage clamp recordings of a single guinea pig smooth muscle at a holding potential of about 60 mV. After treatments with TEA, spontaneous transient inward currents (STIC's) were enhanced, ranging from 30-50 pA. Subsequent bath application of about 100 μM niflumic acid (calcium activated chloride channel blocker) abolishes STIC's. Insert in FIG. 11B shows an enlarged STIC signal before niflumic acid.

FIG. 12 is a graph showing intracellular quenching of MQAE fluorescence by chloride in human airway smooth muscle cells. Bumetanide (10 μM) alone decreases [Cl⁻]ᵢ. Niflumic acid (100 μM) added with bumetanide reduces the bumetanide effect but the net effect is still reduced [Cl⁻]ᵢ concentration from baseline levels. Results shown are representative of 5 trials.

FIG. 13 is a graph showing intracellular calcium concentrations in human airway smooth muscle cells. Cells were treated with thapsigargin to block SR Ca²⁺-ATpase mediated refilling in the absence of extracellular calcium. The re-introduction of 2 mM CaCl₂ induces a rapid increase in [Ca²⁺]ᵢ, indicative of store-operated calcium entry (SOCE). Reduced concentrations of extracellular Cl⁻ concentrations (replaced with gluconate), resulting in depletion of intracellular Cl⁻ concentrations, decreased the magnitude of SOCE. Measurements shown are representative of 4 trials.

FIG. 14 is a graph showing intracellular quenching of MQAE fluorescence by chloride in human airway smooth muscle cells. Depolarization induced by tetraethylammonium (TEA)-acetate is accompanied by an efflux of chloride causing reduced quenching of MQAE fluorescence. The tracings shown are representative of 5 trials.

FIG. 15 is a cartoon showing the mechanistic hypotheses (dashed lines) of airway smooth muscle relaxation by simultaneous blockade of calcium activated chloride channel (CaCC) and Na⁺—K⁺—Cl⁻ transporter (NKCC). Bumetanide blockade of NKCC blocks intracellular Cl⁻ refilling resulting in reduced extracellular efflux through niflumic-acid insensitive Cl⁻ channels which decreases membrane depolarization impairing RhoA activation and reduced intracellular Cl⁻ available to balance charge generation during Ca²⁺ refilling of SR. Niflumic acid blockade of CaCC induces hyperpolarization which impairs RhoA activation. SR=sarcoplasmic reticulum; MLC=myosin light chain; CaM=Ca²⁺ calmodulin dependent protein; MLCK=myosin light chain kinase; ROCK=Rho associated protein kinase.

FIG. 16A-16E show airway lumen area measured in peripheral small airways in rat lung slices. FIG. 16A shows representative light micrographs of the same peripheral airway under baseline (Rest) and following contraction induced by acetylcholine (ACh), potassium chloride (KCl), or TEA. FIG. 16B shows a real-time measurement of lumen area from images in FIG. 16A. Arrows indicate time that each image from FIG. 16A was captured. FIG. 16C is a bar graph showing percent decrease in lumen area after treatment with ACh, KCl, or TEA as compared to the sample at rest. All three contractile agonists result in a significant decrease in lumen area. Asterisk (*) indicates p<0.01. n=6. FIG. 16D shows a real-time measurement of lumen area after treatment with KCl, TEA, and niflumic acid (NFA). FIG. 16E is a bar graph showing the percent decrease in lumen area as a result of TEA-induced contraction and with NFA treatment NFA at 100 μM significantly relaxes the peripheral airway contracted with TEA. Asterisk (*) indicates p<0.01. n=6.

FIG. 17A is a line graph showing membrane potential changes in the potentiometric probe FLIPR Blue in human airway smooth muscle cells. FIG. 17B is a bar graph showing the changes in membrane potential. The chloride channel blocker NFA hyperpolarizes the airway smooth muscle cell plasma membrane, favoring cellular relaxation. The K⁺ channel opener NS1619 was used as a positive control for hyperpolarization while TEA chloride and KCl were used to demonstrate depolarization.

FIG. 18 shows a representative tracing of a whole cell electrophysiologic recording of a human airway smooth muscle cell under current clamp. Following depolarization of the plasma membrane with TEA chloride (favoring contraction), NFA reverses membrane potential (favoring smooth muscle cell relaxation).

FIG. 19 shows the structure of the water-soluble sodium salt form of niflumic acid (NFA).

FIG. 20A shows representative muscle force tracings in an organ bath of guinea pig airway smooth muscle relaxed to a greater extent with low concentration (10 μM) of the water soluble form of NFA compared to the hydrophobic form of the parent compound. FIG. 20B is a bar graph showing muscle force at 60 minutes as a percent of initial force after TEA treatment. The water soluble form demonstrates enhanced potency at relaxing ex vivo guinea pig airway smooth muscle.

FIG. 21 shows that chemically modifying niflumic acid (NFA) as a sodium salt to increase water-solublilty retains its ability to inhibit acetylcholine-induced contractions in human airway smooth muscle. Representative muscle force tracings of ex vivo human airway smooth muscle airway strips are shown. In the upper traces, human airway smooth muscle strips were pretreated with 100 μM water-soluble NFA+10 μM bumetanide before repetitive contractions #2 and #4. In the lower traces, human airway smooth muscle strips were pretreated with a vehicle control. In the upper traces, note the loss of magnitude of acetylcholine-induced contraction following pretreatments that recovers following washout of pretreatments, while the magnitude in the lower traces is unaffected. W=washout of buffer in organ baths between contractile challenges and pretreatments.

FIG. 22 shows in vivo prevention of bronchoconstriction with inhaled pretreatment using a combination of a chloride channel (NFA) and chloride transporter (furosemide) antagonist. Methacholine-induced in vivo lung resistance (forced oscillation technique/Flexivent) in a house dust mite (HDM)-sensitized mouse model was used. Five minutes prior to challenge with various concentrations of aerosolized methacholine, the mice were pretreated with an aerosolized combination of 20 μl of 100 μM niflumic acid (NFA) and 100 μM furosemide or aerosolized PBS. n=4

FIG. 23 shows ex vivo airway smooth muscle force in guinea pig tracheal rings. The chloride channel blocker NPPB and the chloride transporter blocker bumetanide were evaluated for their ability to prevent an initial contraction (n=5) (FIG. 23A) or to relax an existing contraction (n=6) (FIG. 23B). Combined pretreatment with NPPB and bumetanide was required to significantly prevent a contraction, while solo therapy with either NPPB or bumetanide was effective in relaxing an existing contraction. *p<0.05, ***p<0.001 compared to vehicle (0.1% DMSO/0.1% ethanol) control.

FIG. 24A-C show that chloride channel blockers relax a tetraethylammonium chloride (TEA) contraction. FIG. 24A shows representative tracing of tension recordings in organ bath with guinea pig tracheal rings contracted with 10 mM TEA. Rings were treated with vehicle (EtOH) (left panel) or compounds (right panel). Compound 1 completely relaxed TEA contraction within 15 minutes. FIG. 24B shows the structures of Compounds 1 and 13. FIG. 24C shows that 100 μM compound 1 and compound 13 relaxed a TEA contraction (23.8±14.9, 61.0±10.1, respectively percent of TEA contraction at 15 minutes). ** p<0.01, ***p<0.001, n=6-9.

FIG. 25 shows that chloride channel blockers relax potassium gluconate contraction. Chloride channel blockers relaxed 80 mM potassium gluconate contractions (compound 1: 61.5%±6.3, compound 13: 68.2%±4.9, NPPB: 56.7%±10.6, NFA: 47.4%±4.6, percent of potassium gluconate contraction at 30 minutes) *p<0.05, **p<0.01,***p<0.001, n=3-6.

FIG. 26A-B show that pretreatment with chloride channel blockers attenuates the initiation of an acetylcholine contraction in guinea pig tracheal rings. FIG. 26A shows representative tracings of tension recordings in organ bath with guinea pig tracheal rings contracted with Ach EC₅₀ dose. Tracheal rings treated with vehicle (left panel) show no attenuation of contraction, while pre-treatment with compound 13 at 100 μM and bumetanide at 10 μM for 15 minutes (right panel) shows attenuation of the initiation of contraction. FIG. 26B shows that pretreatment with a combination of chloride channel blockers and bumetanide attenuated the initiation of an acetylcholine contraction, while treatment with chloride channel blockers or bumetanide alone failed to attenuate the initiation of acetylcholine contractions. Compound 1 (100 μM)/bumetanide (10 μM): 48.9%±3.6, Compound 13 (100 μM)/bumetanide (10 μM): 37.5%±5.8, NPPB (100 μM)/bumetanide (10 μM): 41.5%±6.3. ***p<0.001, n=5.

FIG. 27A-B show that chloride channel blockers relax the maintenance phase of a pre-established acetylcholine contraction in guinea pig tracheal rings. FIG. 27A shows representative tracings of tension recordings of guinea pig tracheal rings contracted with acetylcholine EC₅₀ dose. Treatment with vehicle (0.2% ethanol, or 0.1% ethanol and 0.1% DMSO) (left panel) showed no relaxation, while treatment with compound 13 at 100 μM and bumetanide at 10 μM (right panel) showed relaxation within 30 minutes. FIG. 27B shows that treatment with chloride channel blockers, bumetanide or a combination of chloride channel blockers and bumetanide relaxed an acetylcholine contraction. Compound 13 (60.1%±5.7) relaxation was significantly increased when combined with bumetanide (27.3%±5.6). N=5-8, *p<0.05, **p<0.01, ***p<0.001, #p<0.05 compared to compound alone.

FIG. 28A-B show that chloride channel blockers relax acetylcholine contraction in human airway smooth muscle. FIG. 28A shows a representative panel of tracings of tension recordings of human ASM strips contracted with Ach EC₅₀ dose. Treatment with compound 13 at 100 μM (bottom left panel) or compound 13 in combination with bumetanide 10 μM (bottom right panel) showed significant relaxation within 30 minutes, while treatment with vehicle (top left panel) or bumetanide 10 μM (top right panel) showed no relaxation. FIG. 28B shows that compound 1 or 13 alone (45.4%±6.3, 52.5%±8.1) and in combination with bumetanide (8.2%±11.1, 41.1%±6.7) relaxed acetylcholine contraction, while vehicle or bumetanide treatment failed to relax an acetylcholine contraction. N=8-13, *p<0.05, **p<0.001, ***p<0.001, #p<0.05 compared to compound alone.

FIG. 29 shows that a combination of 50 μM of compound 13 and 10 μM bumetanide potentiates relaxation of acetylcholine contractions in guinea pig tracheal rings by isoproterenol. The EC₅₀ of isoproterenol-mediated relaxation of an acetylcholine contraction was shifted from 5.99×10⁻⁹ M (vehicle) to 4.77×10⁻¹⁰ M with a combination of 50 μM compound 13 and 10 μM bumetanide (p<0.01). 10 μM bumetanide or 50 μM compound 13 by themselves did not significantly shift relaxation by isoproterenol (2.51×10⁻⁹, 2.7×10⁻⁹ respectively, p>0.05)

FIG. 30A-B show that chloride channel blockers hyperpolarize human airway smooth muscle cells. FIG. 30A shows representative tracing of continuous fluorescence recordings with a FLIPR dye. Potassium gluconate depolarizes, while NS1619 (K⁺-channel opener), compound 1 and niflumic acid (NFA) hyperpolarize human airway smooth muscle (ASM) cells. Potassium gluconate and NS1619 were used as controls as they are known to depolarize and hyperpolarize, respectively. FIG. 30B shows change in relative fluorescent units (RFU) with chloride channel blockers. 100 μM compound 1 (−240.3+/−35), compound 13 (−264.2+/−40) and NFA (−254.7+/−37) all caused a decrease in membrane potential, while 10 μM bumetanide did not cause a significant change. N=12, *p<0.05, **p<0.01, ***p<0.001

FIG. 31A-B show the effects of compound 13 and bradykinin on Ca²⁺-activated chloride channel activity (spontaneous transient inward current, STICs) from cultured human airway smooth muscle cells. FIG. 31A(i) shows a representative tracing of Ca²⁺-activated chloride channel current (STICs) in response to 10 μM compound 13 in human airway smooth muscles cell. FIG. 31A(ii) shows a representative tracing of Ca²⁺-activated chloride channel current (STICs) in response to 10 μM compound 13 and 30 μM bradykinin in human airway smooth muscle cells. FIG. 31B (i and ii) shows two bar graphs summarizing the effects of compound 13 alone or compound 13 after bradykinin on the amplitude (control 1±0.26, compound 130.44±0.09, n=9) and frequency of STICs (control 1±0.29, compound 130.4±0.1 n=9). Bradykinin plus compound 13 groups (n=5). Amplitude (control 1±0.21 bradykinin 1.79±0.11; bradykinin plus compound 130.46±0.13) frequency (control 1±0.43, bradykinin 2.03±0.85, bradykinin plus compound 0.4±0.14). *p<0.05, ** p<0.01 all compared to control groups. Values expressed as fold change with control normalized to 1.

### DETAILED DESCRIPTION OF THE INVENTION

One embodiment of the present invention is a method of treating or ameliorating the effects of a disease characterized by altered smooth muscle contractility. This method comprises administering to a patient suffering from such a disease an effective amount of a calcium-activated chloride channel (CaCC) modulator and a sodium-potassium-chloride co-transporter (NKCC) modulator.

As used herein, in relation to a disease, the term “characterized by” means one of the characteristics or one of the symptoms of the disease. The term “altered” means different from the norm (i.e. the population at large or an individual not suffering from such a disease). The term “smooth muscle” refers to a group of non-striated muscles, generally found in the walls of hollow organs of the body (except the heart), including but not limited to the blood vessels, the respiratory tract, the gastrointestinal tract, the bladder, or the uterus. Preferably, the smooth muscle is airway smooth muscle. The term “contractility” refers to properties associated with the contraction (e.g., of smooth muscle), such as contraction and relaxation of smooth muscles. The contraction and relaxation of smooth muscles is usually not under voluntary control.

As used herein, a “CaCC modulator” is a substance that changes the activity or the opening or the closing of a calcium-activated chloride channel. Preferably, the CaCC modulator of the present invention is a CaCC inhibitor. As used herein, “a CaCC inhibitor” means a substance that acts directly or indirectly on the CaCC to reduce or completely arrest its function, such as, e.g., to close the channel. The CaCC inhibitor may be selected from any known or to be discovered compound or composition having the above described function. Preferably, the CaCC inhibitor is selected from the group consisting of niflumic acid, 5-nitro-2-(3-phenylpropylamino)-benzoate (NPPB), talnifumate, flufenamic acid, 4,4′-diisothiocyanatostilbene-2,2′-disulfonate (DIDS), indanyloxyacetic acid 94 (IAA-94), tamoxifen, 4-acetamido-4′-isothiocyanatostilbene-2,2′-disulfonic acid (SITS), anthracene-9-carboxylic acid (A9C), diphenylamine-2-carboxyl acid (DPC), 6-t-butyl-2-(furan-2-carboxamido)-4,5,6,7-tetrahydrobenzo[b]thiophene-3-carboxylic acid (CaCCᵢₙₕ-A01), 2-hydroxy-4-(4-p-tolylthiazol-2-ylaminobenzoic acid (CaCCᵢₙₕ-B01), morniflumate (Sanofi-Aventis, France), calcium-sensitive chloride channel antagonist (Takeda Pharmaceutical Co. Ltd., Japan),

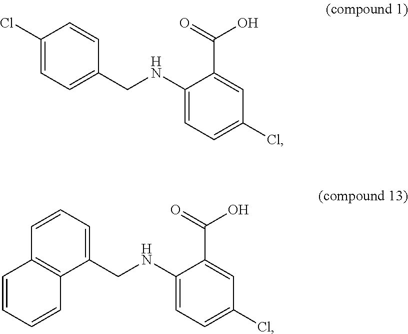 [SMILES: O=C(O)c1cc(Cl)ccc1NCc1ccc(Cl)cc1 ; O=C(O)c1cc(Cl)ccc1NCc1cccc2ccccc12]

a pharmaceutically acceptable salts thereof, and combinations thereof. Other forms of these drugs, including pro-drug forms, whether or not specifically identified herein, are also contemplated. Preferably, the CaCC inhibitor is niflumic acid, or a pharmaceutically acceptable salt thereof. Also preferably, the CaCC inhibitor is NPPB. In another preferred embodiment, the CaCC inhibitor is

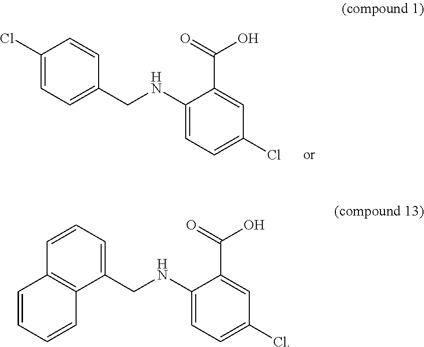 [SMILES: O=C(O)c1cc(Cl)ccc1NCc1ccc(Cl)cc1 ; O=C(O)c1cc(Cl)ccc1NCc1cccc2ccccc12]

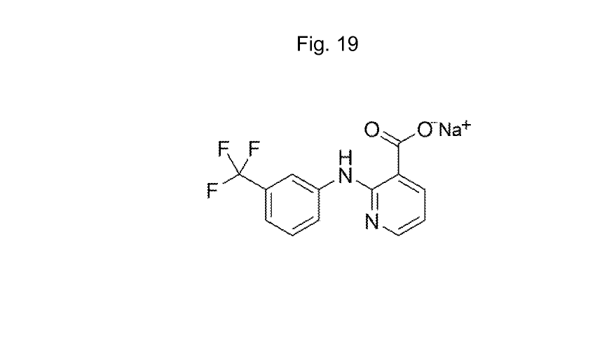

As used herein, a “pharmaceutically acceptable salt” means a salt of the compounds of the present invention which is pharmaceutically acceptable, as defined herein, and which possesses the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. Thus, in one preferred embodiment, the CaCC inhibitor is the sodium salt of niflumic acid shown in FIG. 19:

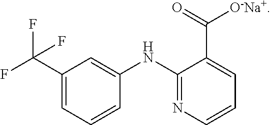

As used herein, a “NKCC modulator” is a substance that changes the activity of the NKCC. Preferably, the NKCC modulator is a NKCC inhibitor. A “NKCC inhibitor” is a substance that acts directly or indirectly to abolish or decrease the activity of the NKCC. The NKCC inhibitor may be selected from any known or to be discovered compound or composition having the above-described function. Preferably, the CaCC inhibitor is selected the group consisting of bumetanide, furosemide, torasemide, azosemide, piretanide, tripamide, etozoline and its metabolite ozolinone, cicletanine, ethacrynic acid, muzolimine, LR-14-890 (Menarini, Italy), lemidosul (Sanofi-Aventis, France), M-12285 (Mochida, Japan), alilusem (Mochida, Japan), sulosemide sodium (Sano-Aventis, France), BTS-39542 (Abbott Laboratories, Abbott Park, Ill.), AY-31906 (Pfizer, New York, N.Y.), brocrinat (Sanofi-Aventis), SA-9000 (Santen, Japan), A-52773 (Abbott Laboratories), A-53385 (Abbott Laboratories), CL-301 (Chlorion Pharma, Canada), Abbott-49816 (Abbott Laboratories), ethacrynic acid (Telor Ophthalmic Pharmaceuticals, Wilmington, Mass.), pharmaceutically acceptable salts thereof, and combinations thereof. Other forms of these drugs, including pro-drug forms, whether or not specifically identified herein, are also contemplated. In another preferred embodiment, the NKCC inhibitor is selected from the group consisting of bumetanide, furosemide, pharmaceutically acceptable salts thereof, and combinations thereof. More preferably, the NKCC inhibitor is bumetanide.

In one aspect of this embodiment, the disease characterized by altered smooth muscle contractility is selected from the group consisting of asthma, chronic obstructive pulmonary disease (COPD), cystic fibrosis, adult respiratory distress syndrome, and bronchospasm, such as those precipitated by noxious stimuli including instrumentation (tracheal intubation), inhaled irritants, drugs or perioperative complications (aspiration, infection, trauma). Other non-limiting examples of diseases characterized by altered smooth muscle contractility include hypertension, bladder spasms, and pre-term labor. Preferably, the disease is asthma or COPD, and the patient is human.

In another aspect of this embodiment, the CaCC modulator and the NKCC modulator are administered as part of a pharmaceutical composition. In the pharmaceutical composition, one or more CaCC modulator(s) are present together with one or more NKCC modulator(s). The exact physical form of the pharmaceutical composition is not critical. Thus, the CaCC and NKCC modulator(s) may be intermixed, physically separated, or otherwise formulated to achieve the desired clinical outcome. Preferably, the pharmaceutical composition is in a unit dosage form.

In this embodiment, the pharmaceutical composition may be co-administered with a β-agonist. In the present invention, “co-administration” includes administration of a pharmaceutical composition comprising a CaCC modulator and a NKCC modulator along with one or more β-agonist(s) together in the same composition, simultaneously in separate compositions, or as separate compositions administered at different times, as deemed most appropriate by a physician.

Non-limiting examples of a β-agonist according the present invention include albuterol, levalbuterol, salmeterol, formoterol, isoproterenol, pirbuterol, and combinations thereof. Co-administration of the pharmaceutical composition comprising a CaCC modulator and a NKCC modulator with a β-agonist leads to synergism (i.e., greater than additive effects). In view of this, lower doses of β-agonist(s) may be used in conjunction with a composition comprising a CaCC modulator and a NKCC modulator, which may result in lower overall side effects.

Another embodiment of the present invention is a pharmaceutical composition for treating or ameliorating the effects of a disease characterized by altered smooth muscle contractility. This composition comprises a pharmaceutically acceptable carrier, a CaCC modulator, and a NKCC modulator. Suitable CaCC modulators and NKCC modulators are as described above. In this embodiment, more than one CaCC and/or NKCC modulator(s) are also contemplated.

Preferably, the disease is selected from the group consisting of asthma, chronic obstructive pulmonary disease (COPD), cystic fibrosis, adult respiratory distress syndrome, and bronchospasm. More preferably, the disease is asthma or COPD. The pharmaceutical composition may be in a unit dosage form. Furthermore, the pharmaceutical composition may be co-administered with a β-agonist, as described above.

Yet another embodiment of the present invention is a method of relaxing airway smooth muscle. This method comprises administering to a patient in need thereof an effective amount of a CaCC modulator and a NKCC modulator. Preferably, the CaCC modulator is a CaCC inhibitor, and the NKCC modulator is a NKCC inhibitor. Suitable CaCC inhibitors and NKCC inhibitors are as exemplified above.

As used herein, “relaxing airway smooth muscle” means reducing the force, tension, or contraction of the smooth muscles related to the portion of the respiratory system through which air flows.

A further embodiment of the present invention is a method of preventing the effects of a disease characterized by altered smooth muscle contractility. This method comprises administering to a patient suffering from such a disease an effective amount of a calcium-activated chloride channel (CaCC) inhibitor and a sodium-potassium-chloride co-transporter (NKCC) inhibitor.

Suitable and preferred CaCC inhibitors, NKCC inhibitors, and diseases are as disclosed herein.

In one aspect of this embodiment, the CaCC inhibitor and the NKCC inhibitor are administered as part of a pharmaceutical composition, which may be in a unit dosage form. Preferably, the pharmaceutical composition is co-administered with a β-agonist, as described above.

An additional embodiment of the present invention is a pharmaceutical composition for preventing the effects of a disease characterized by altered smooth muscle contractility. This composition comprises a pharmaceutically acceptable carrier, a CaCC inhibitor, and a NKCC inhibitor.

Suitable and preferred CaCC inhibitors, NKCC inhibitors, and diseases are as disclosed herein.

In one aspect of this embodiment, the pharmaceutical composition is in a unit dosage form. Preferably, the pharmaceutical composition is co-administered with a β-agonist, as described above.

A further embodiment of the present invention is a method of blocking the onset of an airway contraction. This method comprises administering to a patient in need thereof an effective amount of a CaCC inhibitor and a NKCC inhibitor.

Suitable and preferred CaCC inhibitors, NKCC inhibitors, and diseases are as disclosed herein.

In one aspect of this embodiment, the CaCC inhibitor and the NKCC inhibitor are administered as part of a pharmaceutical composition, which may be in a unit dosage form. Preferably, the pharmaceutical composition is co-administered with a β-agonist, as described above.

Another embodiment of the present invention is a pharmaceutical composition for treating or ameliorating the effects of a disease characterized by altered smooth muscle contractility. This pharmaceutical composition comprises a pharmaceutically acceptable carrier and a CaCC inhibitor selected from the group consisting of

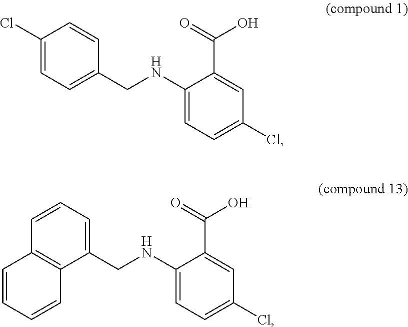 [SMILES: O=C(O)c1cc(Cl)ccc1NCc1ccc(Cl)cc1 ; O=C(O)c1cc(Cl)ccc1NCc1cccc2ccccc12]

a pharmaceutically acceptable salt thereof, and combinations thereof.

Suitable and preferred diseases are as disclosed herein.

In one aspect of this embodiment, the CaCC inhibitor is

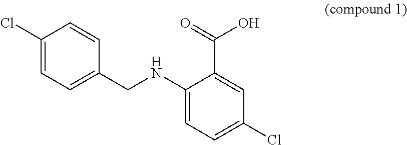 [SMILES: O=C(O)c1cc(Cl)ccc1NCc1ccc(Cl)cc1]

or a pharmaceutically acceptable salt thereof. In another aspect of this embodiment, the CaCC inhibitor is

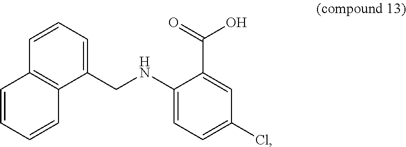 [SMILES: O=C(O)c1cc(Cl)ccc1NCc1cccc2ccccc12]

or a pharmaceutically acceptable salt thereof.

In a further aspect of this embodiment, the CaCC inhibitor is administered as part of a pharmaceutical composition, which may be in a unit dosage form. Preferably, the pharmaceutical composition is co-administered with a β-agonist, as described above.

An additional embodiment of the present invention is a method for treating or ameliorating the effects of a disease characterized by altered smooth muscle contractility. This method comprises administering to a patient suffering from such a disease an effective amount of a CaCC inhibitor selected from the group consisting of

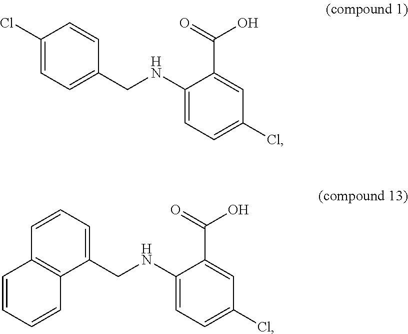 [SMILES: O=C(O)c1cc(Cl)ccc1NCc1ccc(Cl)cc1 ; O=C(O)c1cc(Cl)ccc1NCc1cccc2ccccc12]

a pharmaceutically acceptable salt thereof, and combinations thereof.

Suitable and preferred diseases are as disclosed herein.

In one aspect of this embodiment, the CaCC inhibitor is

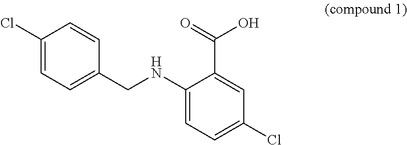 [SMILES: O=C(O)c1cc(Cl)ccc1NCc1ccc(Cl)cc1]

or a pharmaceutically acceptable salt thereof. In another aspect of this embodiment, the CaCC inhibitor is

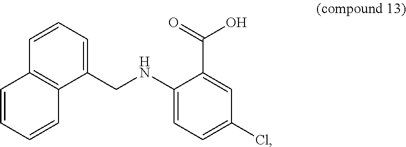 [SMILES: O=C(O)c1cc(Cl)ccc1NCc1cccc2ccccc12]

or a pharmaceutically acceptable salt thereof.

In a further aspect of this embodiment, the CaCC inhibitor is administered as part of a pharmaceutical composition, which may be in a unit dosage form. Preferably, the pharmaceutical composition is co-administered with a β-agonist, as described above.

A further embodiment of the present invention is a method of treating or ameliorating the effects of a disease characterized by altered smooth muscle contractility. This method comprises administering to a patient suffering from such a disease an effective amount of a sodium-potassium-chloride co-transporter (NKCC) inhibitor and a calcium-activated chloride channel (CaCC) inhibitor selected from the group consisting of

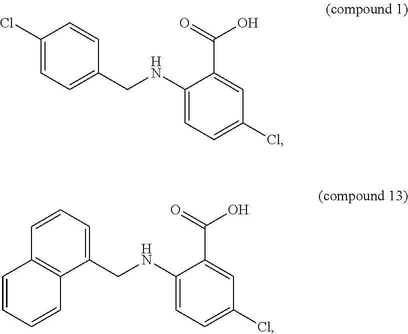 [SMILES: O=C(O)c1cc(Cl)ccc1NCc1ccc(Cl)cc1 ; O=C(O)c1cc(Cl)ccc1NCc1cccc2ccccc12]

a pharmaceutically acceptable salt thereof, and combinations thereof.

Suitable and preferred NKCC inhibitors and diseases are as disclosed herein.

In one aspect of this embodiment, the CaCC inhibitor is

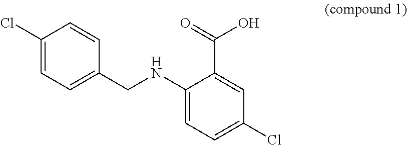 [SMILES: O=C(O)c1cc(Cl)ccc1NCc1ccc(Cl)cc1]

or a pharmaceutically acceptable salt thereof. In another aspect of this embodiment, the CaCC inhibitor is

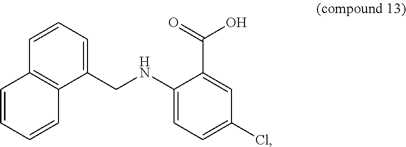 [SMILES: O=C(O)c1cc(Cl)ccc1NCc1cccc2ccccc12]

or a pharmaceutically acceptable salt thereof.

In a further aspect of this embodiment, the CaCC inhibitor and the NKCC inhibitor are administered as part of a pharmaceutical composition, which may be in a unit dosage form. Preferably, the pharmaceutical composition is co-administered with a β-agonist, as described above.

Another embodiment of the present invention is a method of treating or ameliorating the effects of a disease characterized by altered smooth muscle contractility. This method comprises administering to a patient suffering from such a disease an effective amount of a CaCC modulator, which is:

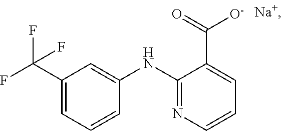 [SMILES: O=C([O-])c1cccnc1Nc1cccc(C(F)(F)F)c1]

and a NKCC modulator.

In one aspect of this embodiment, the NKCC modulator is a NKCC inhibitor. Suitable and preferred NKCC inhibitors are as disclosed herein. Furthermore, suitable and preferred disease are as disclosed herein.

In a further aspect of this embodiment, the CaCC inhibitor and the NKCC inhibitor are administered as part of a pharmaceutical composition, which may be in a unit dosage form. Preferably, the pharmaceutical composition is co-administered with a β-agonist, as described above.

An additional embodiment of the present invention is a pharmaceutical composition for treating or ameliorating the effects of a disease characterized by altered smooth muscle contractility. This composition comprises a pharmaceutically acceptable carrier, a CaCC modulator, which is

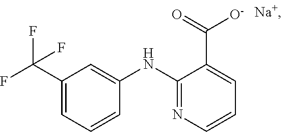 [SMILES: O=C([O-])c1cccnc1Nc1cccc(C(F)(F)F)c1]

and a NKCC modulator.

In one aspect of this embodiment, the NKCC modulator is a NKCC inhibitor. Suitable and preferred NKCC inhibitors are as disclosed herein. Furthermore, suitable and preferred disease are as disclosed herein.

The pharmaceutical composition may be in a unit dosage form. Furthermore, the pharmaceutical composition may be co-administered with a β-agonist, as described above.

In the present invention, an “effective amount” is an amount sufficient to effect beneficial or desired clinical results. An effective amount can be administered in one or more doses. In terms of treatment, an “effective amount” of a CaCC modulator or a NKCC modulator is an amount sufficient to treat or ameliorate the effects of a disease characterized by altered smooth muscle contractility. Detection and measurement of these indicators of efficacy are disclosed below.

An effective amount is generally determined by a physician on a case-by-case basis and is within the skill of one in the art. Several factors are typically taken into account when determining an appropriate dosage. These factors include age, sex and weight of the patient, the condition being treated, the severity of the condition and the form of the drug being administered.

Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of animal, and like factors well known in the arts of medicine and veterinary medicine. In general, a suitable dose of a CaCC modulator or a NKCC modulator according to the invention will be that amount of the compound, which is the lowest dose effective to produce the desired effect. The effective dose of a CaCC modulator or a NKCC modulator may be administered as two, three, four, five, six or more sub-doses, administered separately at appropriate intervals throughout the day, with the proviso that the doses of the CaCC and NKCC modulator simultaneously block the CaCC and NKCC if both CaCC and NKCC modulators are used.

A compound or pharmaceutical composition of the present invention may be administered in any desired and effective manner. Preferably, the compound or pharmaceutical composition of the present invention is administered to a patient in need thereof through a mucosal lining, by, e.g., a nasal or pulmonary spray.

Thus, compounds and pharmaceutical compositions according to the present invention may be administered in an aqueous solution as a nasal or pulmonary spray and may be dispensed in spray form by a variety of methods known to those skilled in the art. Exemplary systems for dispensing liquids as a nasal spray are disclosed in U.S. Pat. No. 4,511,069. The formulations may be presented in multi-dose containers, for example in the sealed dispensing system disclosed in U.S. Pat. No. 4,511,069. Additional aerosol delivery forms may include, e.g., compressed air-, jet-, ultrasonic-, and piezoelectric nebulizers, which deliver the compound or pharmaceutical composition according to the present invention dissolved or suspended in a pharmaceutical solvent, e.g., water, ethanol, or a mixture thereof.

For example, a nebulizer may be selected on the basis of allowing the formation of an aerosol of a modulator disclosed herein. The delivered amount of a modulator provides a therapeutic effect for the diseases disclosed herein. The nebulizer may deliver an aerosol comprising a mass median aerodynamic diameter from about 2 microns to about 5 microns with a geometric standard deviation less than or equal to about 2.5 microns, a mass median aerodynamic diameter from about 2.5 microns to about 4.5 microns with a geometric standard deviation less than or equal to about 1.8 microns, and a mass median aerodynamic diameter from about 2.8 microns to about 4.3 microns with a geometric standard deviation less than or equal to about 2 microns. In other instances, the aerosol can be produced using a vibrating mesh nebulizer. An example of a vibrating mesh nebulizer includes the PARI E-FLOW™ nebulizer or a nebulizer using PARI eFlow technology. More examples of nebulizers are provided in U.S. Pat. Nos. 4,268,460; 4,253,468; 4,046,146; 3,826,255; 4,649,911; 4,510,929; 4,624,251; 5,164,740; 5,586,550; 5,758,637; 6,644,304; 6,338,443; 5,906,202; 5,934,272; 5,960,792; 5,971,951; 6,070,575; 6,192,876; 6,230,706; 6,349,719; 6,367,470; 6,543,442; 6,584,971; 6,601,581; 4,263,907; 5,709,202; 5,823,179; 6,192,876; 6,644,304; 5,549,102; 6,083,922; 6,161,536; 6,264,922; 6,557,549; and 6,612,303; all of which are hereby incorporated by reference in their entireties. More commercial examples of nebulizers that can be used with the CaCC modulators and the NKCC modulators described herein include Respirgard II™, Aeroneb™, Aeroneb™ Pro, and Aeroneb™ Go produced by Aerogen; AERx™ and AERx Essence™ produced by Aradigm; Porta-Neb™, Freeway Freedom™ Sidestream, Ventstream and 1-neb produced by Respironics, Inc. (Murrysville, Pa.); and PARI LC-PIus™, PARI LC-Start, produced by PARI Respiratory Equipment Inc. (Midlothian, Va.). By further non-limiting example, U.S. Pat. No. 6,196,219, is hereby incorporated by reference in its entirety.

Suitable, non-limiting examples of dosages of a CaCC modulator and/or a NKCC modulator according to the present invention administered, e.g., via a nebulizer to an adult human may be from about 0.1 mg/m²/day to 100 mg/m²/day, such as from about 0.5 mg/m²/day to about 80 mg/m²/day, including from about 1 mg/m²/day to about 50 mg/m²/day, about 1 mg/m²/day to about 20 mg/m²/day, about 1 mg/m²/day to about 10 mg/m²/day, about 1 mg/m²/day to about 7 mg/m²/day, or about 3 mg/m²/day to about 7 mg/m²/day. Other representative dosages of a modulator of the present invention include about 0.1 mg/m²/day, 0.2 mg/m²/day, 0.3 mg/m²/day, 0.4 mg/m²/day 0.5 mg/m²/day, 0.6 mg/m²/day, 0.7 mg/m²/day, 0.8 mg/m²/day, 0.9 mg/m²/day, 1 mg/m²/day, 2 mg/m²/day, 3 mg/m²/day, 4 mg/m²/day, 5 mg/m²/day, 6 mg/m²/day, 7 mg/m²/day, 8 mg/m²/day, 9 mg/m²/day, 10 mg/m²/day, 11 mg/m²/day, 12 mg/m²/day, 13 mg/m²/day, 14 mg/m²/day, 15 mg/m²/day, 16 mg/m²/day, 17 mg/m²/day, 18 mg/m²/day, 19 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, 75 mg/m²/day, 80 mg/m²/day, 85 mg/m²/day, 90 mg/m²/day, 95 mg/m²/day, or 100 mg/m²/day. Dosages may be reduced in a child. As noted above, the effective dose of a modulator may be administered as two, three, four, five, six or more sub-doses, administered separately at appropriate intervals throughout the day, with the proviso that the doses of the CaCC and NKCC modulators simultaneously block the CaCC and NKCC.

Nasal and pulmonary spray solutions of the present invention typically comprise the modulators or pharmaceutical composition to be delivered, optionally formulated with a surface-active agent, such as a nonionic surfactant (e.g., polysorbate-80), and one or more buffers. In some embodiments of the present invention, the nasal spray solution further comprises a propellant. The pH of the nasal spray solution is optionally between about pH 3.0 and 6.0, such as 5.0.+/−0.3. Suitable buffers for use with the modulators or pharmaceutical compositions are as described herein or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thiomersal, chlorobutanol, benzylalkonimum chloride, and the like. Suitable surfactants include, but are not limited to, oleic acid, sorbitan trioleate, polysorbates, lecithin, phosphotidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

Within alternate embodiments, mucosal formulations of the present invention may be administered as dry powder formulations comprising the CaCC and NKCC modulators or pharmaceutical compositions according to the present invention in a dry, usually lyophilized, form of an appropriate particle size, or within an appropriate particle size range, for intranasal delivery. Minimum particle size appropriate for deposition within the nasal or pulmonary passages is often about 0.5 μm mass median equivalent aerodynamic diameter (MMEAD), commonly about 1 μm MMEAD, and more typically about 2 μm MMEAD. Maximum particle size appropriate for deposition within the nasal passages is often about 10 μm MMEAD, commonly about 8 μm MMEAD, and more typically about 4 μm MMEAD. Intranasally respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI), which rely on the patient's breath, upon pulmonary or nasal inhalation, to disperse the power into an aerosolized amount. Alternatively, the dry powder may be administered via air-assisted devices that use an external power source to disperse the powder into an aerosolized amount, e.g., a piston pump.

Dry powder devices typically require a powder mass in the range from about 1 mg to 20 mg to produce a single aerosolized dose (“puff”). If the required or desired dose of the compound or pharmaceutical composition according to the present invention is lower than this amount, the powdered active agent will typically be combined with a pharmaceutical dry bulking powder to provide the required total powder mass. Preferred dry bulking powders include sucrose, lactose, dextrose, mannitol, glycine, trehalose, human serum albumin (HSA), and starch. Other suitable dry bulking powders include cellobiose, dextrans, maltotriose, pectin, sodium citrate, sodium ascorbate, and the like.

To formulate compositions for mucosal delivery within the present invention, the compound or pharmaceutical composition according to the present invention can be combined with various pharmaceutically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about 1/3 to 3, more typically 1/2 to 2, and most often 3/4 to 1.7.

The CaCC and NKCC modulators or pharmaceutical compositions of the present invention may be dispersed in a base or vehicle, which may comprise a hydrophilic compound having a capacity to disperse the compounds or compositions of the present invention and any desired additives. The base may be selected from a wide range of suitable carriers, including but not limited to, copolymers of polycarboxylic acids or salts thereof, carboxylic anhydrides (e.g. maleic anhydride) with other monomers (e.g. methyl (meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc. can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the compound or composition according to the present invention.

The CaCC and NKCC modulators or pharmaceutical compositions of the present invention can be combined with the base or carrier according to a variety of methods, and release of the CaCC and NKCC modulators or pharmaceutical compositions of the present invention may be by diffusion, disintegration of the carrier, or associated formulation of water channels. In some circumstances, the active agent(s) is/are dispersed in microcapsules (microspheres) or nanocapsules (nanospheres) prepared from a suitable polymer, e.g., isobutyl 2-cyanoacrylate and dispersed in a biocompatible dispersing medium applied to the nasal mucosa, which yields sustained delivery and biological activity over a protracted time.

To further enhance mucosal delivery of CaCC and NKCC modulators or pharmaceutical compositions of the present invention, formulations comprising such agents may also contain a hydrophilic low molecular weight compound as a base or excipient. Such hydrophilic low molecular weight compounds provide a passage medium through which a water-soluble active agent, such as a physiologically active peptide or protein, may diffuse through the base to the body surface where the active agent is absorbed. The hydrophilic low molecular weight compound optionally absorbs moisture from the mucosa or the administration atmosphere and dissolves the water-soluble active peptide. The molecular weight of the hydrophilic low molecular weight compound is generally not more than 10,000 and preferably not more than 3,000. Exemplary hydrophilic low molecular weight compounds include polyol compounds, such as oligo-, di- and monosaccarides such as sucrose, mannitol, sorbitol, lactose, L-arabinose, D-erythrose, D-ribose, D-xylose, D-mannose, trehalose, D-galactose, lactulose, cellobiose, gentibiose, glycerin and polyethylene glycol. Other examples of hydrophilic low molecular weight compounds useful as carriers within the invention include N-methylpyrrolidone, and alcohols (e.g. oligovinyl alcohol, ethanol, ethylene glycol, propylene glycol, etc.) These hydrophilic low molecular weight compounds can be used alone or in combination with one another or with other active or inactive components of the intranasal formulation.

In sum, mucosal administration according to the invention allows effective self-administration of treatment by patients, provided that sufficient safeguards are in place to control and monitor dosing and side effects. Mucosal administration also overcomes certain drawbacks of other administration forms, such as injections, that are painful and expose the patient to possible infections and may present drug bioavailability problems. For nasal and pulmonary delivery, systems for controlled aerosol dispensing of therapeutic liquids as a spray are well known. For example, metered doses of CaCC and NKCC modulators or pharmaceutical compositions of the present invention are delivered by means of a specially constructed mechanical pump valve, U.S. Pat. No. 4,511,069.

In the present invention, other methods of delivery may also be used. Such methods include, for example, administration by oral ingestion, or as an ointment or drop for local administration to the eyes, or for parenteral or other administration in any appropriate manner such as intraperitoneal, subcutaneous, topical, intradermal, rectal, vaginal, sublingual, intramuscular, intravenous, intraarterial, intrathecal, or intralymphatic. Further, a pharmaceutical composition of the present invention may be administered in conjunction with other treatments. A pharmaceutical composition of the present invention may be encapsulated or otherwise protected against gastric or other secretions, if desired.

Suitable, non-limiting examples of dosages of a CaCC modulator according to the present invention administered, e.g., via oral ingestion or via injection, to an adult human may be from about 0.05 mg/day to 20 mg/day, such as from about 0.1 mg/day to about 10 mg/day, including from about 0.5 mg/day to about 2 mg/day. Other representative dosages of a modulator of the present invention include about 0.1 mg/day, 0.2 mg/day, 0.3 mg/day, 0.4 mg/day 0.5 mg/day, 0.6 mg/day, 0.7 mg/day, 0.8 mg/day, 0.9 mg/day, 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, or 20 mg/day. Suitable, non-limiting examples of dosages of a NKCC modulator according to the present invention administered, e.g., via oral ingestion or via topical application, to an adult human may be from about 0.1 g/day to 5 g/day, such as from about 0.25 g/day to about 2 g/day, including from about 0.75 g/day to about 1.5 g/day. Other representative dosages of a modulator of the present invention include about 0.1 g/day, 0.2 g/day, 0.3 g/day, 0.4 g/day 0.5 g/day, 0.6 g/day, 0.7 g/day, 0.8 g/day, 0.9 g/day, 1 g/day, 2 g/day, 3 g/day, 4 g/day, or 5 g/day. Dosages may be reduced in a child. As noted above, the effective dose of a modulator may be administered as two, three, four, five, six or more sub-doses, administered separately at appropriate intervals throughout the day, with the proviso that the doses of the CaCC and NKCC modulators simultaneously block the CaCC and NKCC.

The pharmaceutical compositions of the invention comprise one or more active ingredients, e.g., CaCC and NKCC modulators, in admixture with one or more pharmaceutically-acceptable carriers and, optionally, one or more other compounds, drugs, ingredients and/or materials, including, e.g., β-agonists. Regardless of the route of administration selected, the modulators/pharmaceutical compositions of the present invention are formulated into pharmaceutically-acceptable dosage forms, including unit dosage forms, by conventional methods known to those of skill in the art. See, e.g., Remington, The Science and Practice of Pharmacy (21^{st} Edition, Lippincott Williams and Wilkins, Philadelphia, Pa.).

Pharmaceutically acceptable carriers are well known in the art (see, e.g., Remington, The Science and Practice of Pharmacy (21^{st} Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier used in a pharmaceutical composition of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art. More generally, “pharmaceutically acceptable” means that which is useful in preparing a pharmaceutical composition that is generally safe, non-toxic, and neither biologically nor otherwise undesirable and includes that which is acceptable for veterinary use as well as human pharmaceutical use.

The pharmaceutical compositions of the invention may, optionally, contain additional ingredients and/or materials commonly used in such pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants as disclosed above, such as hydrofluoroalkane, particularly 1,1,1,2-tetrafluoroethane, heptafluoralkane (HFA) such as 1,1,1,2,3,3,3-heptafluoro-n-propane or mixtures thereof, as well as other chlorofluorohydrocarbons and other volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Pharmaceutical compositions suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Pharmaceutical compositions for rectal or vaginal administration may be presented as a suppository, which may be prepared by mixing one or more active ingredient(s) with one or more suitable nonirritating carriers which are solid at room temperature, but liquid at body temperature and, therefore, will melt in the rectum or vaginal cavity and release the active compound. Pharmaceutical compositions which are suitable for vaginal administration also include pessaries, tampons, creams, gels, pastes, foams or spray formulations containing such pharmaceutically-acceptable carriers as are known in the art to be appropriate.

Dosage forms for the topical or transdermal administration include powders, sprays, ointments, pastes, creams, lotions, gels, solutions, patches, drops and inhalants as previously disclosed. The modulators/pharmaceutical compositions may be mixed under sterile conditions with a suitable pharmaceutically-acceptable carrier. The ointments, pastes, creams and gels may contain excipients. Powders and sprays may contain excipients and propellants as previously disclosed.

Pharmaceutical compositions suitable for parenteral administrations comprise one or more of each kind of CaCC and NKCC modulators in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the CaCC and NKCC modulators then depends upon their rates of dissolution which, in turn, may depend upon crystal size and crystalline form. Alternatively, delayed absorption of a parenterally-administered CaCC and NKCC modulators may be accomplished by dissolving or suspending the CaCC and NKCC modulators in an oil vehicle. Injectable depot forms may be made by forming microencapsule matrices of the CaCC and NKCC modulators in biodegradable polymers. Depending on the ratio of the CaCC and NKCC modulators to polymer, and the nature of the particular polymer employed, the rate of CaCC and NKCC modulator release can be controlled. Depot injectable formulations are also prepared by entrapping the modulators in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

The following examples are provided to further illustrate the methods and compositions of the present invention. These examples are illustrative only and are not intended to limit the scope of the invention in any way.

### EXAMPLES

### Example 1

Epithelial denuded guinea pig tracheal rings were suspended in organ baths under 1 g resting tension with continuous digital recordings of muscle force. In separate studies, guinea pig airway smooth muscle cells were enzymatically dispersed and cultured.

Induced electrophysiological changes in membrane potential and current were measured using traditional whole cell patch clamp methods. Immortalized human airway smooth muscle cells were grown to confluence on collagen-treated T25 flasks. Collagenase type IV in SmBM2 medium (Lonza, Walkersville, Md.) was used to release cells adherent to the collagen matrix in the flask. Medium with cells in suspension was then harvested in a 10-ml conical tube and centrifuged at 300×g. Supernatant was removed, and the pellet was resuspended in SmBM2 medium and transferred into collagen-treated glass bottom 1-cm Petri dishes at about 10% confluence. Each dish was then incubated at 37° C. and 5% CO₂ for 1-4 hours for reattachment of cells to glass-bottom Petri dishes.

Glass-bottom dishes served as a disposable recording chamber. ALA VM-8, an 8-chamber pressure-driven drug application system, was used in a still bath of extracellular salt solution. Whole-cell intracellular voltage recordings under current clamp conditions were performed with a 2-kHz Bessel filter, recording at 10 kHz using an Axopatch 200b amplifier (Axon Instruments, Foster City, Calif., USA). Electrodes were pulled using a P-97 micropipette puller from 1.5-mm OD borosilicate capillary glass (Sutter Instruments, Novato, Calif., USA). Glass electrode resistances ranged from 5 to 10 MO with intracellular solution. Whole-cell intracellular current recordings under voltage-clamp conditions were performed with a 2-kHz Bessel filter, recording at 10 kHz using an Axopatch 200b amplifier. For current measurements (FIG. 11) intracellular solutions consisted of (in mM) 140 TEA-Cl, 5 MgATP, 5 EGTA, 1 MgCl₂, 10 HEPES, and 5 CaCl₂ (pH 7.2). Extracellular solution consisted of (in mM) 134 NaCl, 1.4 KCl, 10 HEPES, 1 MgCl₂, 1.8 CaCl₂, and 10 glucose (pH 7.4). This induced the classic spontaneously transient inward currents (STICs) and spontaneously transient outward currents (STOCs) well described in this cell type. STOCs are believed to represent activation of calcium activated potassium (K_{Ca}) channels while STICs are believed to represent activation of CaCCs. TEA-Cl was included in the buffer to enhance STICs which were then recorded during the addition of 100 μM niflumic acid. When recording TEA- and niflumic acid-induced responses on membrane potential (ΔmV), the intracellular solutions contained (in mM) 140 KCl, 5 MgATP, 5 EGTA, 1 MgCl₂, 10 HEPES, and 5 CaCl₂ (pH 7.2), and the extracellular solutions consisted of (in mM) 134 NaCl, 5 MgATP, 5 EGTA, 1 MgCl₂, 10 HEPES, and 5 CaCl₂ (pH 7.2). All recordings were analyzed on Clampfit 8.0 software (Molecular Devices).

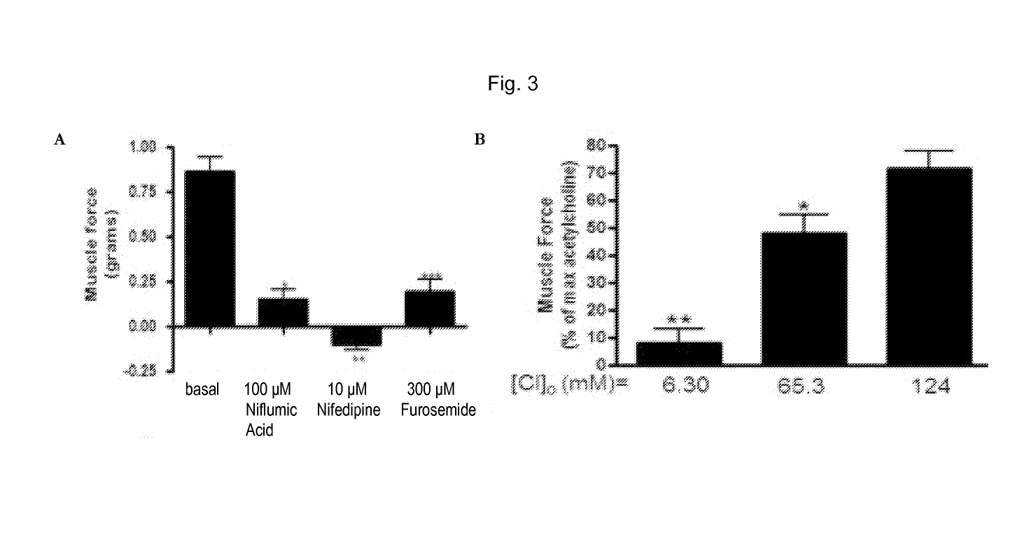

Guinea pig airway smooth muscle contractions induced by membrane depolarization by K channel blockade with 10 mM tetraethylammonium chloride (TEA) were relaxed by the L-type calcium channel blocker nifedipine (111%; p<0.001, n=5), the NKCC blocker furosemide (77.6%, p<0.001, n=4), and the CaCC channel blocker niflumic acid (82.5%, p<0.001 n=5) (FIG. 3A) directly. TEA-induced increase in muscle force was proportional to external buffer chloride concentrations (6.30567 mM, 65.3 mM, 124 mM), an electrochemical gradient in favor of inward chloride movement (7.9±5.5, 48.1±6.9, 71.7±6.3% of control acetylcholine contraction, respectively (N=4-6) (FIG. 3B). In contrast, electrophysiological recordings in isolated airway smooth muscle cells in the presence of TEA revealed a CaCC dependent outward anion flow, with a current/voltage relationship consistent with chloride (FIG. 11).

These functional and electrophysiologic results suggested that a chloride cycle comprised of outward Cl flow through CaCC channels and re-uptake via NKCC is required for maintenance of a depolarized membrane potential and the maintenance of airway smooth muscle tone. Blockade of these chloride regulatory proteins offers potential therapeutic targets.

### Example 2

Human muscle tissue was acquired from excess lung airways trimmed during surgery from healthy lung transplant donors. Acquired tissue was stored overnight at 20° C. Airway smooth muscle contractions measured ex vivo in organ baths were performed as previously described (Gallos et al., 2008; Gallos et al., 2009, Gallos et al., 2011; Gleason et al., 2010; Yim et al., 2011; Mitzuta et al., 2008). Closed guinea pig tracheal rings or strips of human airway smooth muscle (tracheal or main stem bronchus) were suspended in organ baths, which had 95% oxygen constantly perfusing through Dulbecco's Modified Eagle Medium. Rings were cut on the cartilaginous borders of the smooth muscle. The epithelial layer was dissected under microscopic assistance. Briefly, tissues were suspended in a water-jacketed (37° C.) 2-ml organ bath (Radnoti Glass Technology, Monrovia, Calif.) and attached to a Grass FT03 force transducer (Grass Telefactor, West Warwick, R.I.) coupled to a computer via BioPac hardware and Acqknowledge 7.3.3 software (Biopac Systems, Goleta, Calif.). Kreb's-Henseleit (KH) buffer was continuously bubbled with 95% oxygen and 5% carbon dioxide and tissues were allowed to equilibrate at 1 g (guinea pig) or 1.5 g (human) isotonic force for 1 hour with fresh KH buffer changes every 15 minutes.

Following equilibration, in guinea pig experiments, the capsaicin analog N-vanillylnonanamide (10 μM final) was added to the organ baths to first activate and then deplete nonadrenergic, noncholinergic nerves. After N-vanillylnonanamide induced force had returned to baseline (about 50 minutes), the tracheal rings were washed and then subjected to two cycles of increasing cumulative concentrations of acetylcholine (0.1 μM to 0.1 mM) to determine the EC₅₀ concentrations of acetylcholine required for each individual ring. In experiments with human tissue, no vanillylnonanamide pretreatment was done. To avoid bias between treatment groups, tissues were contracted to individually calculated EC₅₀ values for acetylcholine and tissues with similar Eₘₐₓ values were randomly assigned to treatments within individual experiments. Following extensive KH buffer changes (8-9 times) tissues were allowed to stabilize at isotonic resting tension (about 1.0 g). To remove confounding effects of other procontractile pathways, each bath received a complement of antagonists 20 minutes prior to subsequent contractile challenge. The antagonists included pyrilamine (10 μM; H₁ histamine receptor antagonist), and tetrodotoxin (1 μM; blocker of endogenous cholinergic or C-fiber neuronal effects) in guinea pig experiments or pyrilamine and 10 μM MK571 (leukotriene receptor antagonist) in human tissue experiments.

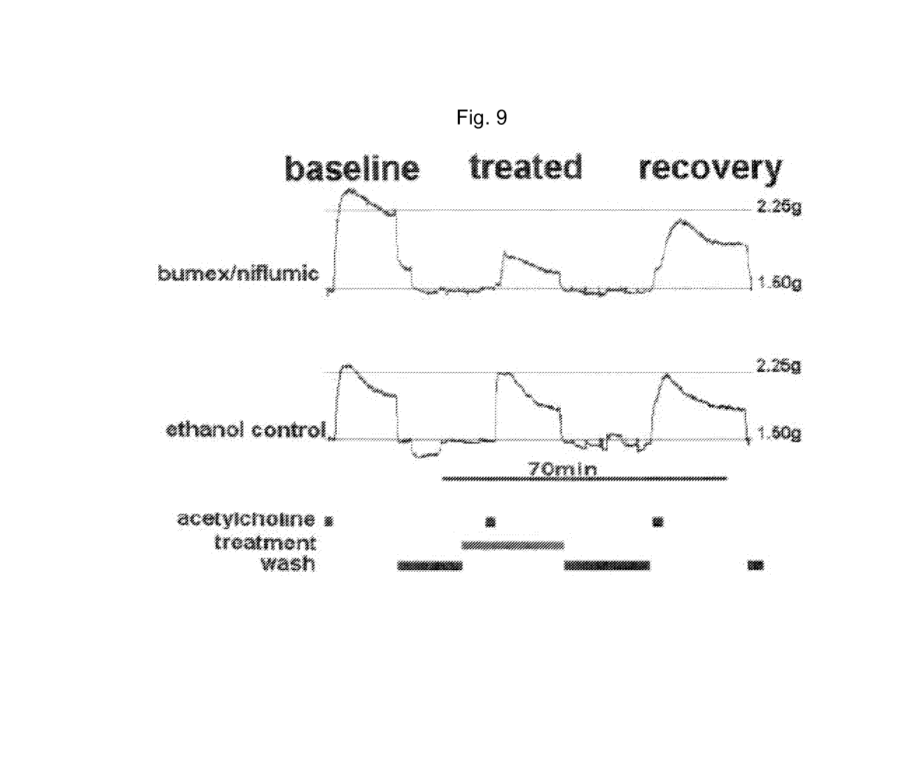

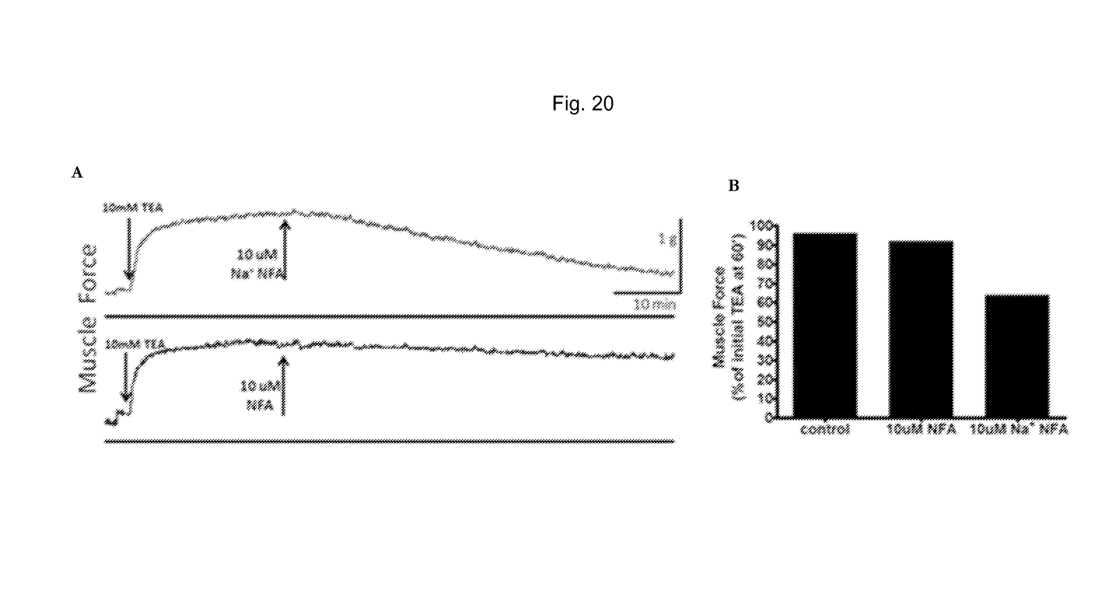

Following these preliminary contractile challenges and pretreatments, one of two paradigms was utilized; a single contractile challenge (e.g. TEA in FIG. 20) or repetitive challenges with an EC₅₀ concentration of acetylcholine, interspersed with buffer changes and redosing of pyrilamine/tetrodotoxin/MK571. For the repetitive challenges control responses were first established. After three control challenges with an EC₅₀ of acetylcholine, tissues were pretreated with either the chloride channel blocker niflumic acid (10-100 μM) alone, the NKCC blockers bumetanide (10 μM) or furosemide (100 μM) alone or a combination of niflumic acid with bumetanide. Following three repetitive acetylcholine challenges in the presence of these blockers, three recovery acetylcholine challenges were performed to determine the reversibility of the blocker effect and confirm functional recovery of smooth muscle contractile function (See, FIG. 9).

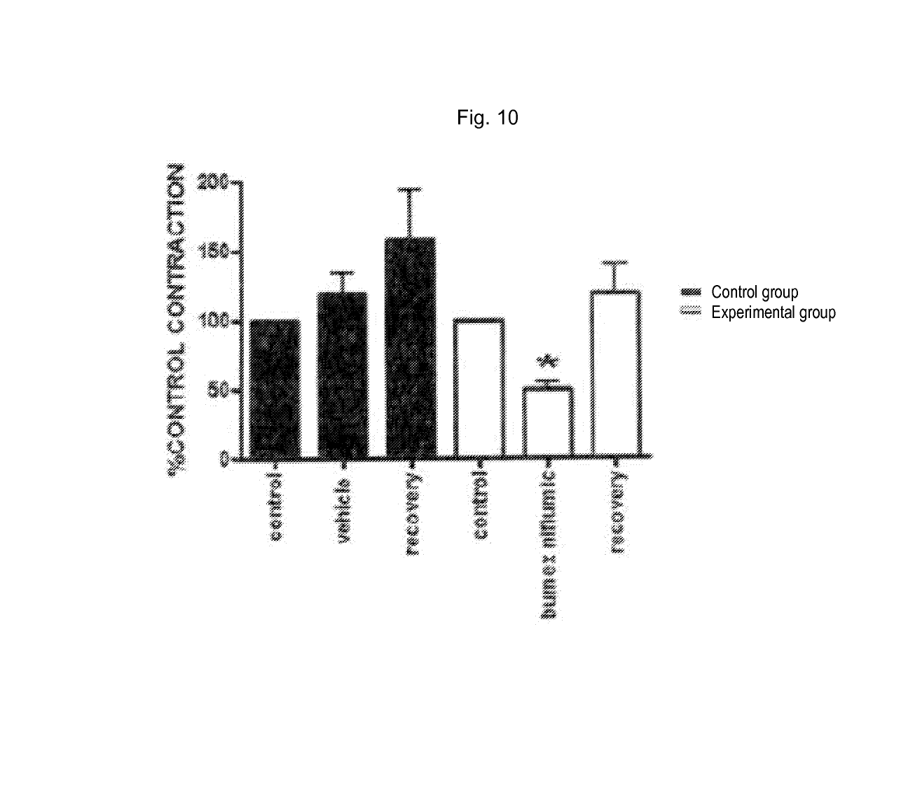

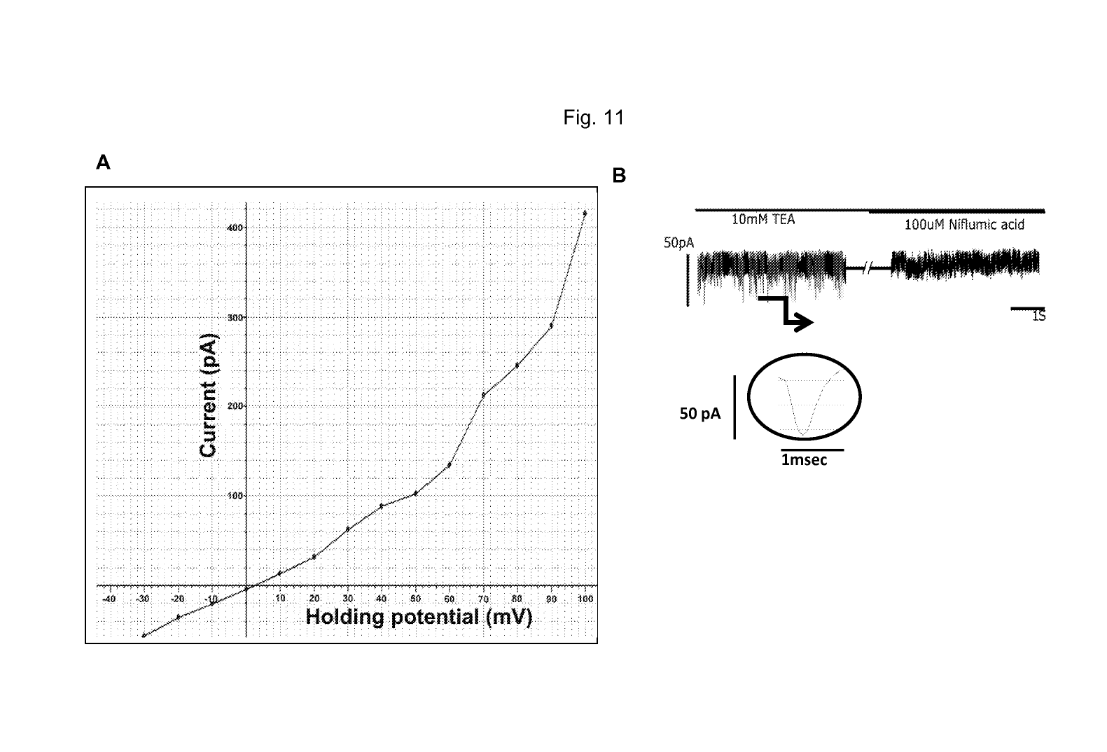

The difference was insignificant between control contractions and the vehicle treatment. Treatments of niflumic acid and bumex showed significant attenuation by 51±5.976% SEM compared to control and was statistically significant (n=3, p<0.05). Recovery contractions, after thorough washing, showed a loss of significant difference as compared to control (See, FIG. 10).

These experiments showed that the combination of calcium activated chloride channel blockade and sodium potassium chloride co-transporter blockade have significant contractile antagonistic effects on human airway smooth muscle in vitro. These data suggest that this combination of drugs deactivates contractile mechanisms involving membrane depolarization and/or membrane potential dependent calcium entry. This combination can flourish as a viable treatment option for airway hyperresponsiveness and can be a viable alternative or an adjunct to β-agonist therapy.

### Example 3

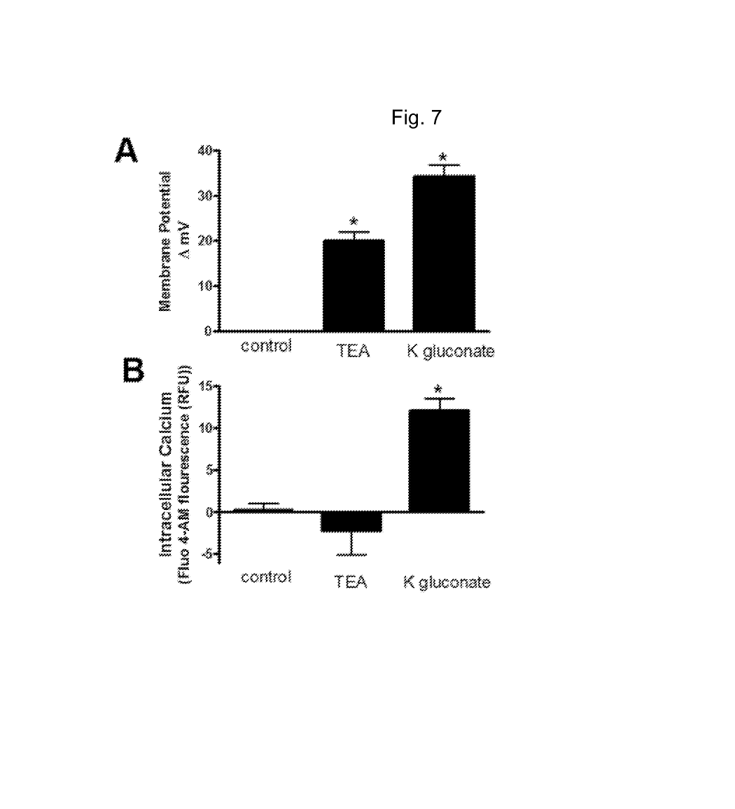

Natural native ligand acetylcholine and salts (K-gluconate and TEA-acetate) will be used for contracting/depolarizing airway smooth muscle. Acetylcholine is a common ligand used in in vitro contraction assays as it is a natural endogenous constrictor of ASM. However, cell signaling events following acetylcholine are very complex including the activation of both Gi (via M2 muscarinic receptors) and Gq (via M3 muscarinic receptors) which in turn activate calcium release from the SR following inositol triphosphate (IP₃) synthesis, inhibition of synthesis of cyclic AMP, activation of the small G proteins including RhoA (modulating calcium sensitivity) and depolarization of the membrane potential. Therefore, dissecting cellular mechanisms using acetylcholine-induced contractions are difficult. To study the isolated effects of membrane potential and increases in intracellular calcium on SR calcium refilling and activation of RhoA, two contractile agonists that are devoid of direct G protein activation will also be used. KCl induces membrane depolarization and increases intracellular calcium while tetraethylammonium (TEA) chloride depolarizes the plasma membrane without an increase in intracellular calcium (FIG. 7). Potassium gluconate and TEA-acetate will be used to retain the depolarizing effects of the cations of these reagents without the confounding effects of adding large concentrations of chloride while studying chloride handling. Thus, K-gluconate and TEA-acetate will be useful to separate out the mechanistic effects of membrane potential and increased intracellular calcium in SR calcium refilling and RhoA activation studies in both isolated native airways and isolated ASM cells.

### Example 4

Simultaneous blockade of the CaCC and NKCC is required to block acetylcholine-induced contraction of airway smooth muscle.

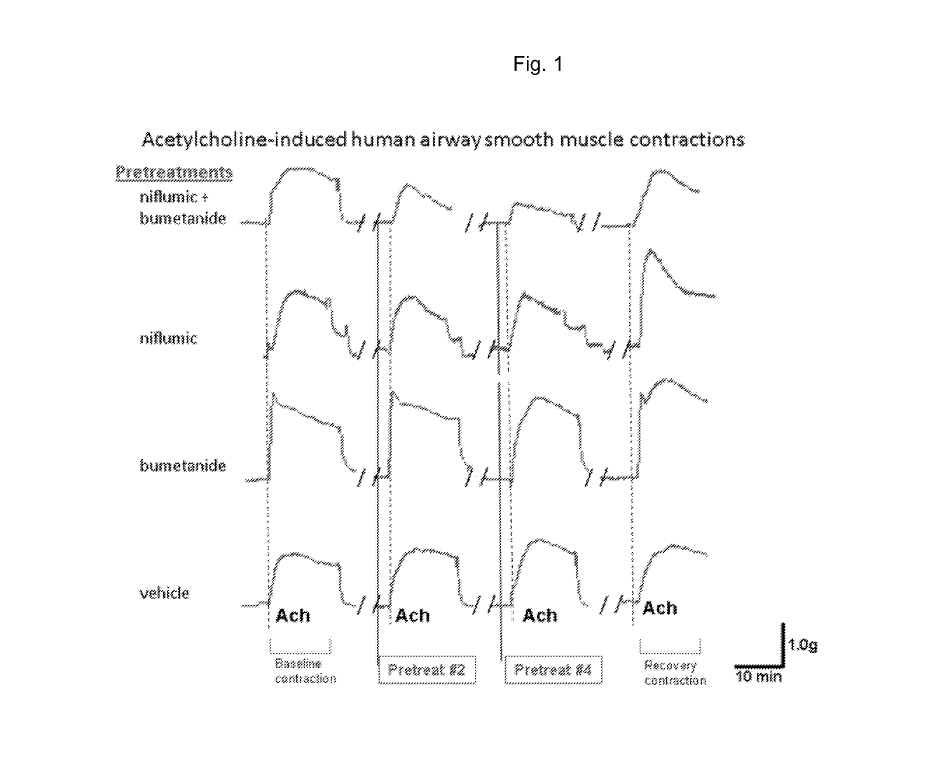

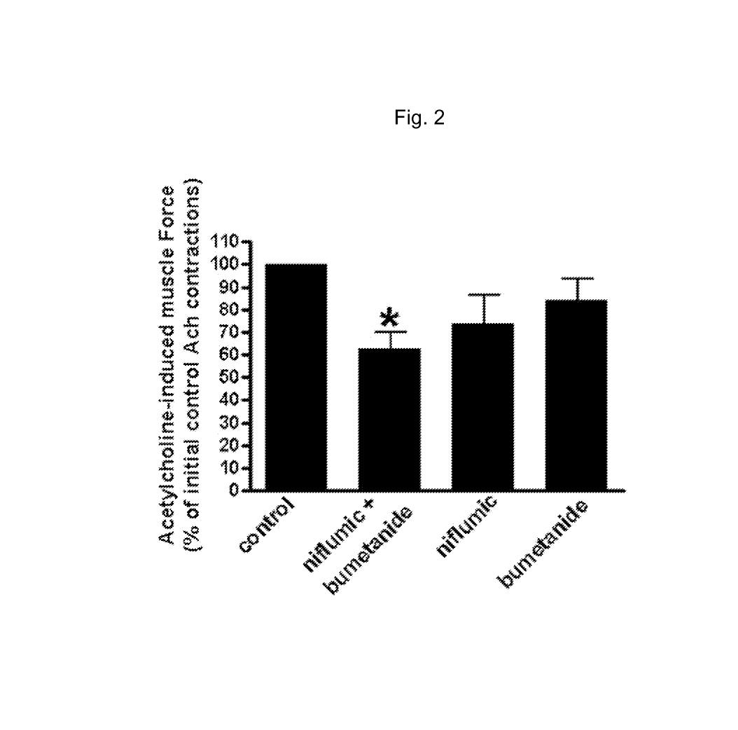

In native ASM strips from human lung transplants, simultaneous pretreatment with blockers of CaCC and NKCC reduces the magnitude of an acetylcholine induced contraction (FIG. 1, top tracings). In contrast, separate blockade of CaCC with niflumic acid or separate blockade of NKCC with bumetanide does not significantly reduce contraction (FIG. 1 and FIG. 2). These results explain why separate blockade of these chloride-handling pathways in ASM cells did not result in significant attenuation of contractions. These results are important because these are functional contractions in native human ASM and these contractions are in response to a classic endogenous constrictor of ASM (acetylcholine).

Contractile studies were then performed in guinea pig tracheal rings using 10 mM tetraethylammonium (TEA) chloride to induce a contraction dependent initially solely on membrane depolarization. It was reasoned that if the hypotheses that niflumic acid hyperpolarized the plasma membrane while another NKCC blocker, furosemide, stabilized membrane potential (resisting depolarization) that pretreatment with either of these drugs alone should attenuate a TEA-induced depolarizing contraction. These hypotheses were confirmed as demonstrated in FIG. 3A.

In vivo airway pressure in guinea pigs was measured as follows. Male Hartley guinea pigs (about 400 g) were anesthetized and instrumented in this established model of airway inflation pressure measurements in protocols previously described (Sunaga et al., 2010, Gleason et al., 2009; Jooste et al., 2007) and approved by the Columbia University Institutional Animal Care and Use Committee. Anesthesia was induced by intraperitoneal injection of urethane (1.5 g/kg) and increased by 0.5 g i.p. until lack of foot pinch response before the start of the procedure. Urethane was chosen for its long duration of action (about 10 hours) and lack of influence on respiratory nerve function. Animals received a tracheostomy with a 1-inch 14-g angiocatheter attached to a microventilator (model 683; Harvard Apparatus, South Natick, Mass.; IMV, volume control, tidal volume 2.6 ml, 66 breaths/min). The ventilator circuit was connected via side ports to two separate pressure monitors with different sensitivities (TSD160B 0-12.5 cmH₂O and TSD160C₀₋₂₅ cmH₂O; Biopac Systems, Goleta, Calif.) using rigid pressure tubing and was continuously monitored and recorded using Acqknowledge software. Animals then received bilateral external jugular catheters by using PE-50 tubing for a continuous succinylcholine infusion (5 mg·kg⁻¹·h⁻¹), started after a bolus of 1.5 mg/kg to remove any influence of chest wall or diaphragm muscle tone on airway pressures, and an independent catheter for delivery of study drugs. A carotid arterial line was placed to monitor blood pressure and heart rate and to ensure adequate depth of anesthesia by monitoring hemodynamic responses. After preparation, each animal received increasing intravenous acetylcholine (4-28 μg/kg, i.v.) until consistent increases in peak pulmonary inflation pressures (Ppi; 50-100% above baseline) were achieved. Animals were then pretreated 12 minutes before repetitive acetylcholine challenges with vehicle (100 μl DMSO, i.v.) or 5 mg niflumic acid i.v. +5 mg furosemide i.v. The optimized acetylcholine dose for each animal was then injected 6 times at 30 second intervals while continuously measuring airway pressure responses (peak pulmonary inflation pressure) and hemodynamics.

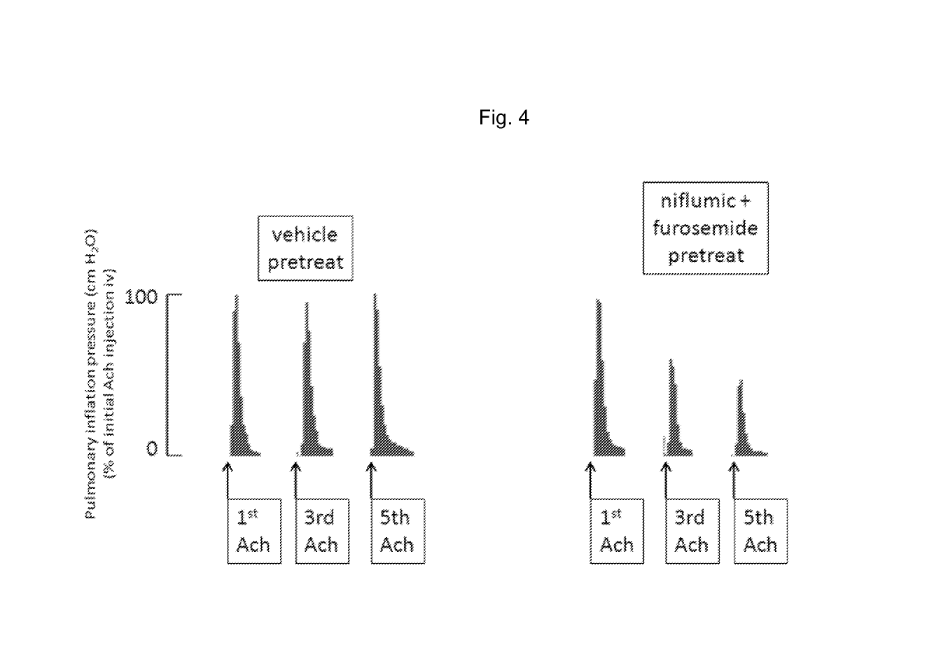

Whether simultaneous blockade of CaCC (with intravenous (i.v.) niflumic acid) and NKCC (with i.v. furosemide) could attenuate in vivo bronchoconstriction was questioned. These studies were performed in a well-characterized in vivo guinea pig model of bronchoconstriction measured by an increase in pulmonary inflation pressures following intravenous-, aerosolized- or vagal nerve-induced bronchoconstriction. As shown in FIG. 4, pretreatment with blockers of both CaCC and NKCC resulted in an attenuation of bronchoconstriction during repetitive challenges with i.v. acetylcholine at 30 second intervals. These results agree with in vitro data demonstrated in isolated airway rings in FIG. 1 and are consistent with the mechanistic hypothesis that blockade of these Cl⁻ pathways interrupts refilling of intracellular Ca²⁺ stores resulting in sequential decreases in contractile forces.

### Example 5

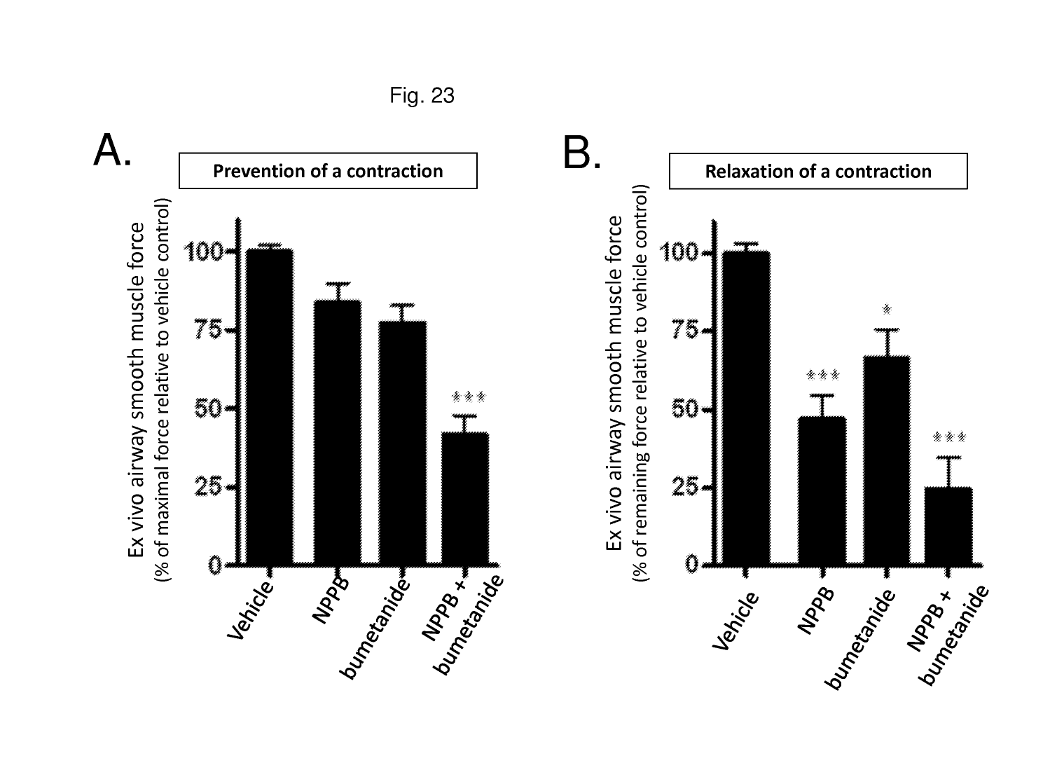

As a proof of concept for chloride channel blockade in general, another chloride channel blocker, 5-nitro-2-(3-phenylpropylamino)-benzoic acid (NPPB) was used in an ex vivo guinea pig airway smooth muscle model. The results of the experiment not only demonstrated that combined therapy with a chloride channel and chloride transporter blocker is required to block the onset of a contraction, but also that an established contraction can be relaxed by using either of these drug classes alone (FIG. 23).

### Example 6

The findings in Example 5 will be also confirmed by using additional blockers of CaCC (alone and in combination with NKCC blockade) in organ bath force measurements in human ASM strips contracted with acetylcholine, K gluconate or TEA. Specifically, NPPB, a CaCC blocker with a broader selectivity for CaCC subtypes, will be compared to niflumic acid. NPPB is believed to block CaCC on both plasma membrane and sarcoplasmic reticulum (SR) membrane while niflumic acid blockade is thought to be limited to plasma membrane CaCC. The results of this comparison will be important in the mechanistic studies to determine a potential role for CaCC on the SR in modulating Cl⁻ influx to balance charge generation during Ca²⁺ refilling of the SR. Additionally, CaCC blockers previously used clinically as anti-inflammatory (diclofenac) or anti-mucus (talnifumate (niflumic acid pro-drug) therapies may also be used.

### Example 7

Bumetanide is clinically used as a diuretic due to blockade of NKCC. To confirm that blockade of NKCC is the mechanism of action responsible for the results disclosed herein, NKCC will be inhibited using furosemide, another clinically used but chemically distinct NKCC blocker in organ bath force measurements (alone and in combination with CaCC blockade) in native ASM from humans and guinea pigs contracted with acetylcholine, K gluconate or TEA.

Although acetylcholine is a classic constrictor of ASM contraction in vivo, additional contractile agonists contribute to in vivo contraction in humans including histamine, tachykinins and leukotrienes. One effective combination of CaCC and NKCC inhibitor against these alternative contractile mediators will be used in native ASM of human and guinea pig to determine whether simultaneous blockade of CaCC and NKCC block ASM contraction induced by a wide range of contractile mediators.

ASM rings will be flash frozen from experimental paradigms outlined above for the measurement of RhoA activation via a rhotekin pull-down assay and myosin light chain phosphorylation via immunoblotting.

Guided by the results in isolated native guinea pig airway rings, the effects of separate and simultaneous blockade of CaCC and NKCC against contractile mediators will be measured in guinea pigs in vivo. A well-established model of pulmonary inflation measurements will be used in urethane-anesthetized guinea pigs in response to intravenous, aerosolized or vagal nerve induced smooth muscle constriction. Following the establishment of a repetitive baseline response to intravenous or aerosolized acetylcholine or histamine or vagal nerve stimulation, blockers of CaCC (e.g. niflumic acid, diclofenac, talnifumate) plus blockers of NKCC (furosemide, bumetanide) will be given as pretreatments (intravenously or by aerosol) before subsequent re-challenge with the contractile mediators.

### Example 8

The following experiments demonstrate that the mechanism(s) of relaxation induced by modulation of the chloride cycle at the plasma membrane is mediated by changes in membrane potential and intracellular chloride concentrations, which in turn impair refilling of the sarcoplasmic reticulum with calcium and calcium sensitization of the contractile apparatus of human airway smooth muscle. The experiments show that hyperpolarization of the plasma membrane by blockade of CaCCs results in reduced depolarization-induced activation of rhoA resulting in reduced phosphorylation of myosin light chain in both native human airway smooth muscle and cultured human airway smooth muscle. They also show that reduced intracellular concentrations of chloride due to blockade of NKCCs results in impaired refilling of the sarcoplasmic reticulum with calcium due to insufficient balance of charge generation normally accomplished by chloride influx from the cytosol to SR.

The mechanistic underpinnings of the effects of separate and simultaneous blockade of CaCC and NKCC on membrane potential, chloride flux, store-operated calcium entry (SOCE) and RhoA activation/calcium sensitivity will be studied in freshly isolated native ASM from human and guinea pig and freshly isolated and cultured human ASM cells.

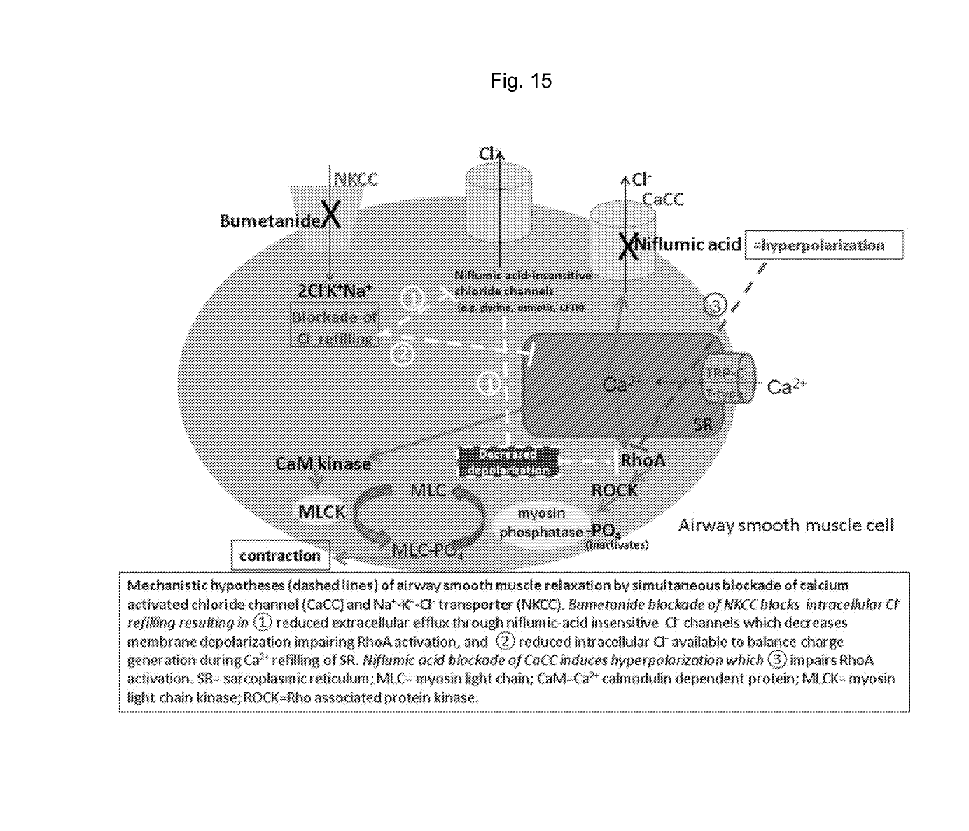

Freshly isolated native and cultured human ASM cells will be used for the measurement of membrane potential (by both classic current clamp whole cell recordings and the potentiometric fluorescent probe FLIPR), calcium activation assays (fluorescent fluo4-AM), chloride flux assays (fluorescent MQAE assays) and RhoA activation assays (rhotekin-bead binding of GTP-bound RhoA). The mechanism to be addressed in this Example is summarized in FIG. 15.

### Example 9

It is believed that the mechanisms of blockade of acetylcholine contraction in human ASM by simultaneous blockade of CaCC and NKCC is due to effects on cellular chloride and membrane potential. These effects on membrane potential in turn modulate both SR calcium refilling and activation of RhoA, which modulates calcium sensitivity.

To determine whether blockade of CaCCs or NKCCs induce membrane potential changes in cultured human airway smooth muscle cells, the FLIPR in vitro fluorescent dye assay (Molecular Devices) was used as described by Wafford et al. (2009). Briefly, human airway smooth muscle cells were grown to 100% confluence in 96-well black-walled plates and were washed with warmed (37° C.) normal-chloride buffer [consisting of (in mM) 130 sodium chloride, 2 CaCl₂, 1 MgCl₂, 10 D-glucose, and 10 HEPES, pH 7.4] four times. A stock solution (100% dye) of FLIPR blue dye (Molecular Devices) was prepared by reconstitution of 1 vial (125 mg) with 100 ml of the normal-chloride buffer (assay buffer). A 50% working stock was prepared by further diluting the reconstituted blue dye 1:1 with assay buffer and was used to load cells (90 μl/well) over 20 minutes at 37° C. All reagents were dissolved in assay buffer. Baseline fluorescence was measured for 3 minutes prior to the first control additions (assay buffer). Three minutes later, airway smooth muscle cells were exposed to 100 μM niflumic acid, 10 μM bumetanide, 10 mM tetraethylammonium (TEA) chloride (K⁺ channel blocker), 40 mM KCl (depolarization stimulus), 10 μM NS1619 (K⁺ channel opener), 60 mM potassium gluconate (depolarization stimulus), or appropriate vehicles (0.1% ethanol for niflumic acid and bumetanide). The fluorescence produced by membrane potential change following solution additions was quantified after subtracting changes induced by assay buffer alone.

To determine whether blockade of CaCCs or NKCCs induce changes in intracellular chloride concentrations in human airway smooth muscle cells, the MQAE in vitro fluorescent dye assay (Invitrogen) was used. Briefly, human airway smooth muscle cells were grown to 100% confluence in 96-well black-walled plates. Cells were loaded overnight with 10 mM MQAE in serum-free basal cell culture medium (M199, Invitrogen) and were washed once with warmed (37° C.) normal-chloride buffer [consisting of (in mM) 130 sodium chloride, 2 CaCl₂, 1 MgCl₂, 10 D-glucose, and 10 HEPES, pH 7.4]. Fluorescence was measured at 2 second intervals (excitation/emission 350/460 nm) before and after the addition of 10 mM TEA-acetate, 100 μM niflumic acid, 10 μM bumetanide, or a combination of niflumic acid/bumetanide or appropriate vehicles. An increase in fluorescence represents decreased intracellular chloride (removal of halide quenching of MQAE) while decreased fluorescence represents increased intracellular chloride (enhanced halide quenching of MQAE).

To demonstrate that not all depolarizing stimuli result in an increase in intracellular calcium and that extracellular chloride concentrations influence the magnitude of store-operated calcium entry (SOCE), human airway smooth muscle cells plated on 96-well plates were incubated in 100 μl Hanks' balanced salt solution (HBSS) (in mM: NaCl 138, KCl 5, CaCl₂ 2.5, MgSO₄ 0.4, MgCl₂ 0.5, Na₂HPO₄ 0.34, NaHCO₃ 4.2, KH₂PO₄ 0.44, D-glucose 5.5, HEPES 20, pH 7.4) containing 5 μM Fluo-4 AM (DMSO vehicle final concentration of 0.5%), 0.05% Pluronic® F-127 (DMSO vehicle final concentration of 0.25%) and 2.5 mM probenecid for 30 minutes at 37° C. Cells were washed twice with HBSS containing 2.5 mM probenecid and left for further 30 minutes at room temperature to allow complete de-esterification of the intracellular AM esters. This buffer was exchanged at 100 μl/well just before starting the measurement of fluorescence. The fluorescence was then continuously recorded every 5 seconds at wavelengths of 485 nm excitation and 528 nm emission using a microplate reader (FlexStation III, Molecular Devices). Fluorescence was measured before and after 10 mM TEA-acetate or 40 mM potassium gluconate to compare different depolarizing stimuli. In separate experiments the effect of extracellular chloride concentrations on SOCE was measured by the predepletion of extracellular calcium in low or high chloride buffer (55 mM versus 110 mM NaCl, osmotically balanced with Na gluconate) in the presence of thapsigargin to deplete intracellular calcium stores. Calcium induced fluorescence changes were measured with the reintroduction of 2 mM extracellular CaCl₂.

Measurement of activated rhoA in cultured human airway smooth muscle cells were conducted as follows. Confluent cultured human airway smooth muscle cells in 10 cm² dishes were washed once with warm Hanks balanced salt solution (HBSS) and then stimulated for various time points with 10 mM TEA-acetate or 60 mM potassium gluconate. Reaction was stopped by solubilizing cells in buffer. Following clearance of non-solubilized cell material, the solubilized cells were reacted with Rhotekin bound to beads, which isolated activated RhoA from total RhoA. Fractions were then analyzed on a commercially available ELISA plate that quantitates activated versus total RhoA (Cytoskeleton, Inc.)

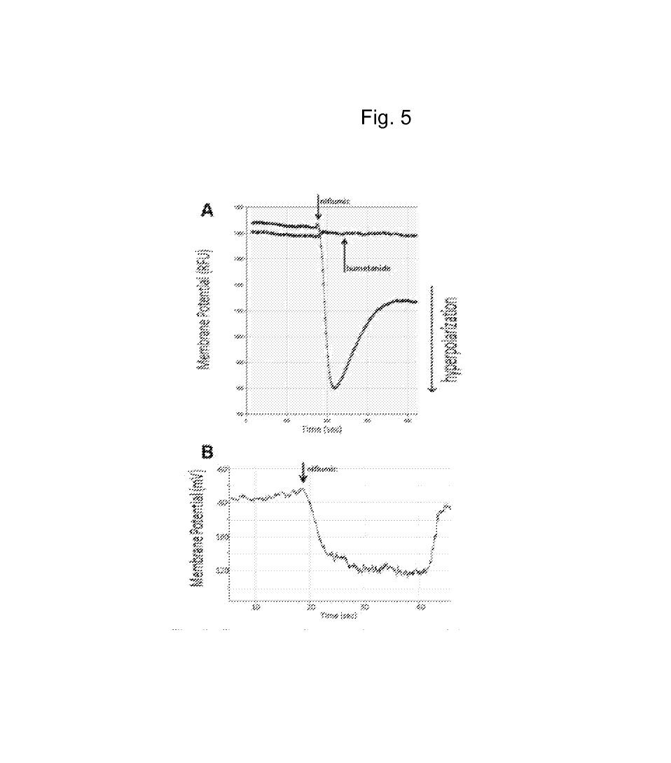

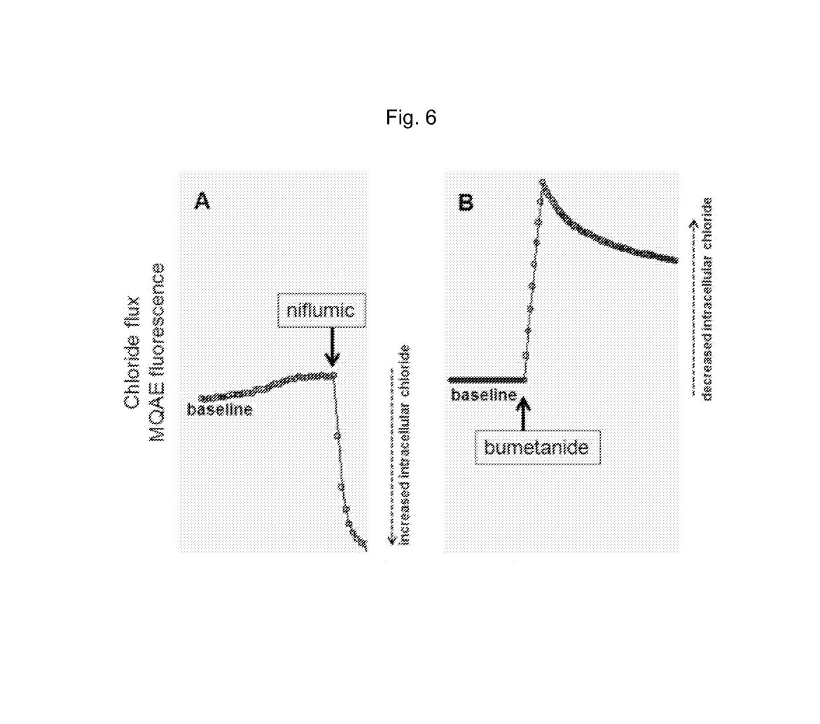

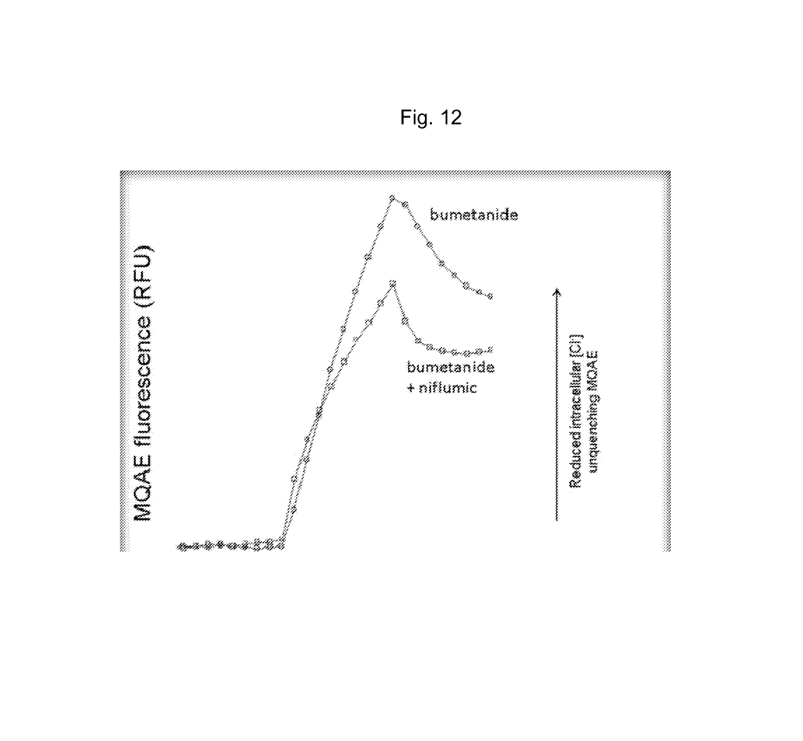

In this example, it has been demonstrated in human ASM cells that blockade of CaCC with niflumic acid hyperpolarizes the plasma membrane (demonstrated by both whole cell current clamp and FLIPR potentiometric fluorescent probe assays) (FIG. 5) while blocking efflux of cellular chloride (demonstrated by enhanced quenching of the chloride sensitive fluorescent probe, MQAE) (FIG. 6A). Furthermore, demonstrated herein is that treatment of human ASM cells with bumetanide stabilizes membrane potential (FIG. 5A) and impairs chloride entry (FIG. 6B). These opposing effects of CaCC blockade and NKCC blockade on intracellular chloride concentrations (FIG. 6) is consistent with the hypothesis but raises an additional question: if the mechanism of NKCC blockade favoring muscle relaxation involves a reduced intracellular chloride concentration impairing charge balance in the SR during Ca²⁺ refilling, then the combined treatment with niflumic acid and bumetanide should still yield a reduced intracellular chloride concentration. This indeed occurred as demonstrated in FIG. 12.

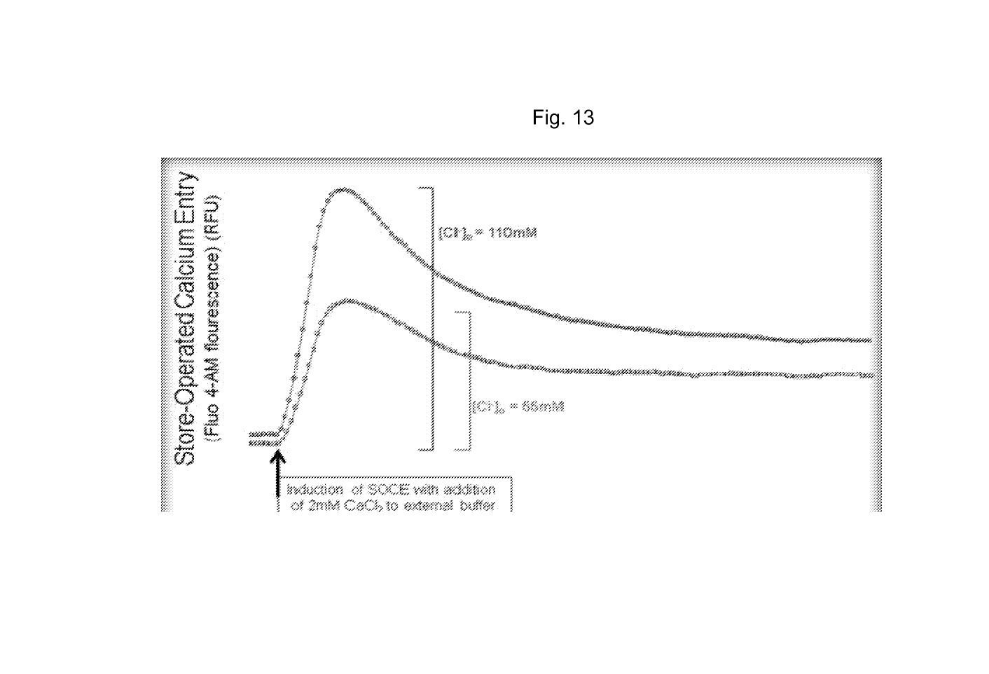

It is believed that this reduced intracellular concentration of chloride reduces the amount of cytosolic chloride available to influx into the SR to balance charge generation during Ca²⁺ refilling (Janssen, 2002). Thus, store-operated calcium entry (SOCE) was induced by pretreating human ASM cells with thapsigargin in Ca²⁺-free buffer. In parallel wells, 50% of the external chloride was replaced with gluconate. This resulted in a decrease in the magnitude of SOCE consistent with the hypothesis that reduced extracellular chloride reduces intracellular chloride, which in turn reduces charge balance and the ability of the SR to refill with Ca²⁺ (FIG. 13).

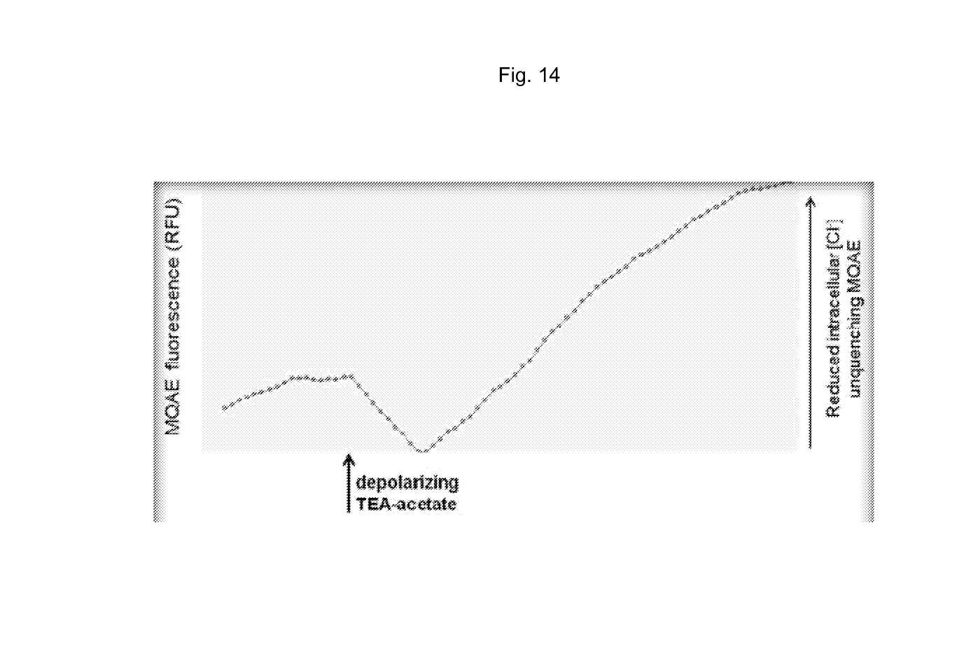

The results shown in FIG. 7A demonstrated that K-gluconate and TEA depolarized ASM cells as measured in a whole cell configuration under current clamp. However, K-gluconate but not TEA elevated intracellular Ca²⁺ as measured by fluo4-AM (FIG. 7B). Furthermore, TEA-acetate results in decreased intracellular chloride concentrations (FIG. 14), consistent with the hypothesis that depolarization includes efflux of Cl⁻. It is believed that this is due to the degree of membrane depolarization induced by high concentrations of K gluconate (60-75 mM) as opposed to low concentrations of TEA (10 mM). This suggests that the threshold for membrane depolarization is lower than the threshold for increases in intracellular calcium. This will be further clarified in dose response studies that will evaluate the dose responses of KCl, K-gluconate and TEA-acetate for membrane potential and intracellular calcium effects. The ability of low concentrations of TEA to depolarize without increasing Ca²⁺ will be exploited to address the hypothesis that activation of rhoA signaling (and thus enhanced Ca²⁺ sensitivity) can occur independent of Ca²⁺.

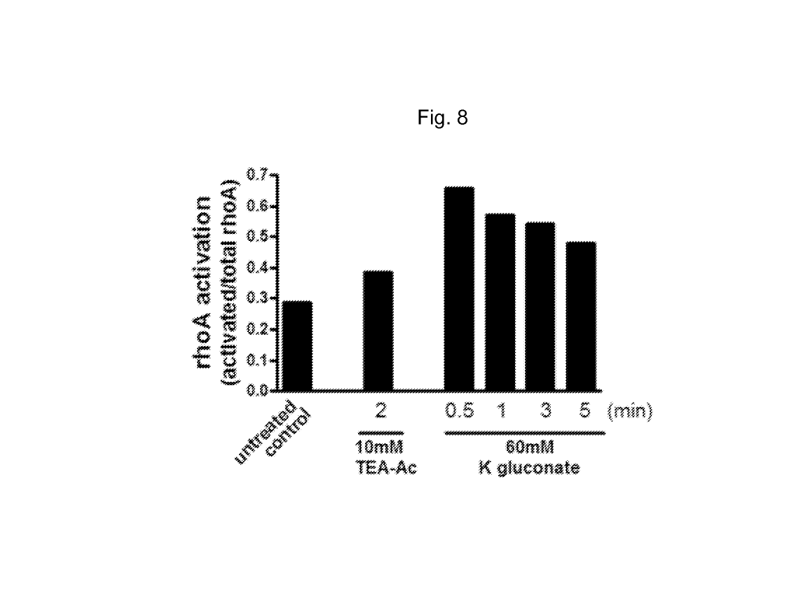

The results shown in FIG. 8 demonstrated that this low depolarizing concentration of TEA which does not increase intracellular Ca²⁺, activates rhoA.

### Example 10

Further experiments are directed at demonstrating that blockade of CaCC hyperpolarizes the cell membrane, and that blockade of NKCC resists depolarization of the cell membrane. In turn, this combined resistance to depolarization impairs activation of rhoA which classically increases the sensitivity of the contractile proteins to calcium.

Membrane potential and intracellular Cl⁻ concentrations [Cl⁻]; will be measured in cultured human ASM cells in response to varying concentrations (1 nM-1mM) of inhibitors of the CaCC (niflumic acid, flufenamic acid, 4,4′-Diisothiocyanatostilbene-2,2′-disulfonate (DIDS), indanyloxyacetic acid 94 (IAA-94), NPPB) using both fluorescent potentiometric probes (FLIPR) and classical whole cell recordings under current clamp conditions. These studies are expected to confirm that CaCC blockade hyperpolarizes the plasma membrane and increases [Cl⁻]ᵢ. Furthermore, two of these inhibitors (NPPB and DIDS) block more members of the CaCC family than niflumic acid and will address the hypothesis that the additive effect of NKCC blockade is the removal of internal chloride that continues to efflux through niflumic-insensitive channels.

Membrane potential and [Cl⁻]ᵢ will be measured in cultured human ASM cells pretreated with varying concentrations (1 nM-1 mM) of two clinically utilized inhibitors of the NKCC (furosemide and bumetanide) before attempting to depolarize the cell with 10 mM TEA using both fluorescent potentiometric probes (FLIPR) and classical whole cell recordings under current clamp conditions. These studies are expected to confirm that NKCC blockade decreases [Cl⁻], and resists depolarization of the plasma membrane.

[Cl⁻]ᵢ will be measured in cultured human ASM cells while reducing [Cl⁻]ₒ (replaced equimolar with gluconate). These experiments are expected to confirm that reducing [Cl⁻]ₒ reduces [Cl⁻]ᵢ, an important control for the following experiment.

Store-operated calcium entry (SOCE) will be measured by standard methods in human ASM cells (thapsigargin treatment in Ca²⁺-free external buffer with reintroduction of external Ca²⁺). SOCE will be measured with either blockade of NKCC or by varying the concentration of [Cl⁻]ₒ (replaced with equimolar gluconate). These experiments are expected to confirm that reducing [Cl⁻], by either reductions in [Cl⁻]ₒ or NKCC blockade result in reductions in SOCE.

A second and not mutually exclusive mechanism to explain the smooth muscle relaxation effect of membrane potential, is a direct link to rhoA activation and a change in the Ca²⁺ sensitivity of the ASM cell. rhoA activation will be measured via a rhotekin binding assay in both native and cultured human ASM cells and native guinea pig airways. The same samples will also be assayed for myosin light chain phosphorylation (a distal signaling event of rhoA activation) by immunoblotting. Native or cultured ASM will be subjected to depolarization without elevating intracellular Ca²⁺ (10 mM TEA-acetate) or depolarization with elevation of intracellular Ca²⁺ (60 mM K gluconate). These studies are expected to establish (1) a link between membrane depolarization and rhoA activation (and thus calcium sensitivity), and (2) the dependence of increases in intracellular Ca²⁺ for rhoA activation.

### Example 11

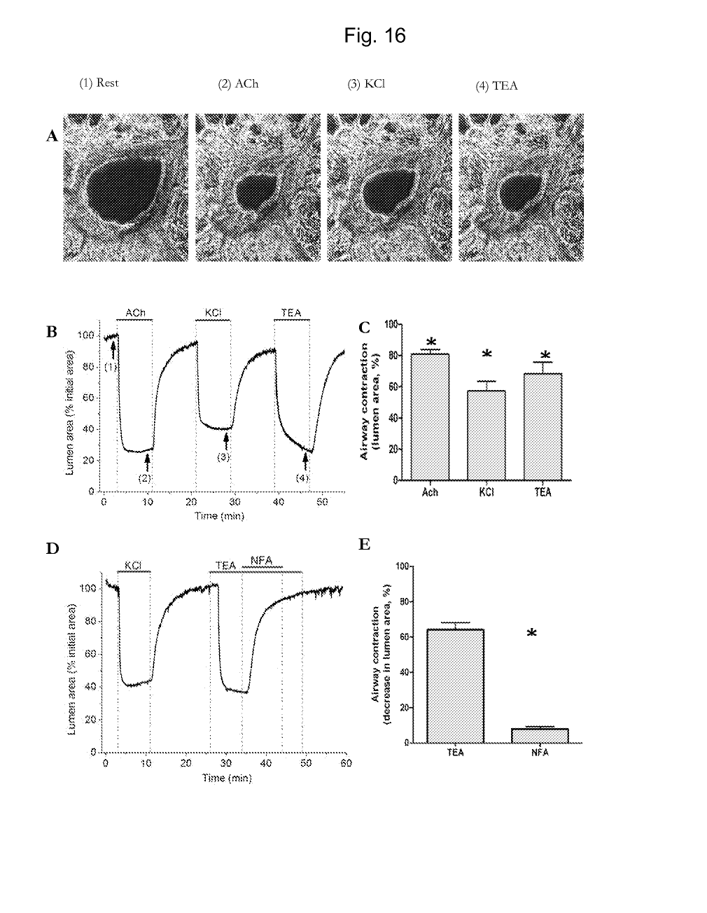

Airway smooth muscle is distributed along the branching bronchioles of the lung down to the level of the respiratory bronchioles. It is believed that the airway smooth muscle of the small airways is most important in bronchoconstriction in asthma. Relaxant effects of niflumic acid were confirmed in small peripheral airways in lung slices (FIGS. 16D and E). The results shown in FIG. 16 demonstrated that blockade of chloride channels in small airways, directly visualized in lung slices, reverses constriction induced by a depolarizing stimulus, such as tetraethylammonium chloride. These findings further augment the therapeutic feasibility of this class of drugs in relaxing the small airways thought most important in asthma.

### Example 12

The measurement of airway contraction of a luminal area of small lung airways using the ex vivo rat lung slice method was performed as described (Perez-Zoghbi et al., 2007). The trachea was cannulated with an intravenous catheter, and after the chest cavity was opened, the collapsed lungs were reinflated with 1.3±0.1 ml of 2% agarose (low-gelling temperature) in Sigma-Hanks' Balanced Salt solution (sHBSS), followed by the injection of 0.2±0.1 of air to flush the agarose-sHBSS out of the airways and into the distal alveolar space. Subsequently, a warm (37° C.) solution of gelatin (type A, porcine skin, 300 bloom, 6% in sHBSS) was perfused through the intrapulmonary blood vessels via the pulmonary artery by injecting about 0.3 ml into the right ventricle. The warm agar and gelatin were gelled with cold sHBSS. A single lung lobe was removed and cut into serial sections of about 130 μm thick with a vibratome at about 4° C., starting at the lung periphery. Slices were maintained in DMEM (Invitrogen) at 37° C. and 10% CO₂ for up to 3 days. At 37° C., the gelatin in the blood vessel lumen dissolved, leaving the blood vessel lumen empty. Lung slices were mounted in a custom-made perfusion chamber and held in place with a small sheet of nylon mesh. A second cover glass edged with silicone grease was placed over the lung slice. Perfusion of the lung slice was performed using a gravity-fed perfusion system. The volume of the chamber was about 100 μl with a perfusion rate of about 800 μl/min. For phase-contrast microscopy, the lung slice was observed with an inverted microscope with a 10× objective, and images were recorded using a charge-coupled device camera and image acquisition software (Video Savant, 10 Industries Inc., Ontario, Canada). Digital images were recorded in time lapse (30 frames/min). The area of the bronchiole and arteriole lumen was calculated, from each image, by pixel summing using custom-written software. Experiments were performed at room temperature.

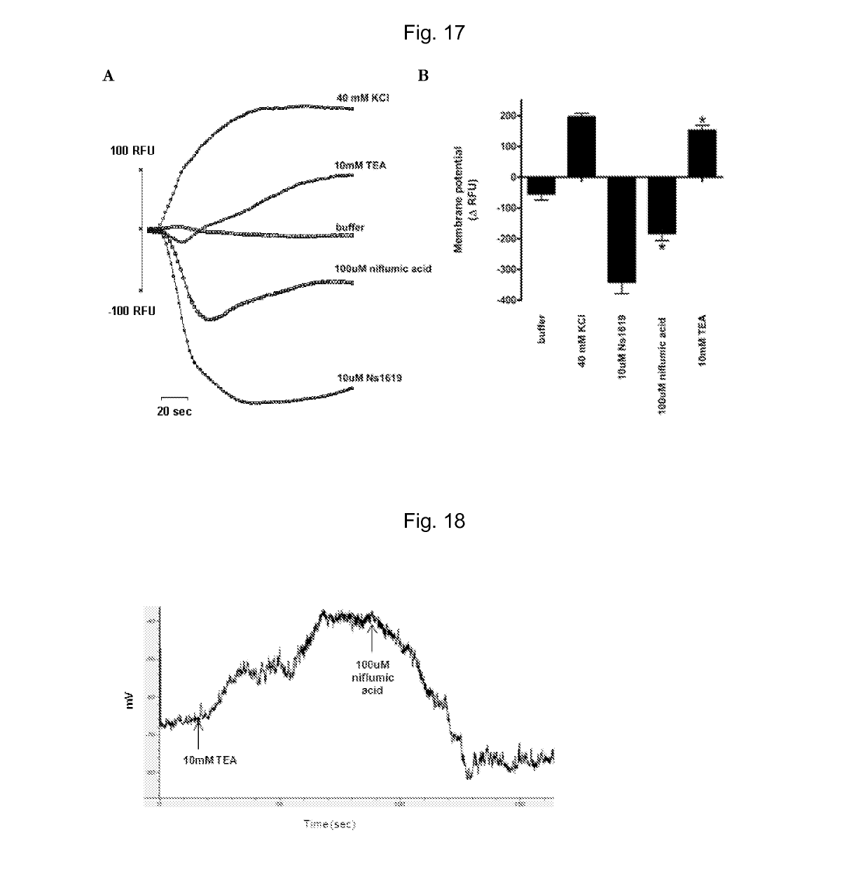

The functional relaxation of airway smooth muscle by chloride channel blockade is thought to include a change in membrane potential (hyperpolarization) which reverses depolarization induced by contractile agonists. FIG. 17 demonstrates that the chloride channel blocker niflumic acid (NFA) hyperpolarizes cell plasma membrane potential using a fluorescent dye that detects membrane potential. FIG. 18 shows this hyperpolarizing effect another way by using traditional whole cell, current clamp electrophysiology methods in human airway smooth muscle cells.

### Example 13

Traditional therapeutic delivery of effective compounds for bronchoconstriction in asthma occurs via nebulized or dry powder inhalation to the lungs. A limitation of the parent niflumic acid (NFA) compound used in previous examples was its poor water solubility, making it sub-optimal for formulations used in inhalation therapy. To remedy this, a water-soluble form of NFA was synthesized, the structure of which is given in FIG. 19.

To synthesize this water-soluble form of NFA, the parent compound (niflumic acid, Sigma-Aldrich N0630, St. Louis, Mo.) was exposed to 10% methanolic potassium hydroxide for 16 hours, followed by thin layer chromatography to confirm salt conversion. Sample was then processed via nuclear magnetic resonance (NMR) to confirm the absence of any unanticipated byproduct formation.

Airway smooth muscle contractions measured ex vivo in organ baths were performed as set forth above. Muscle force measurements taken from organ baths of airway smooth muscle relaxed with water-soluble or hydrophobic NFA demonstrated enhanced potency of water-soluble NFA at relaxing airway smooth muscle (FIG. 20).

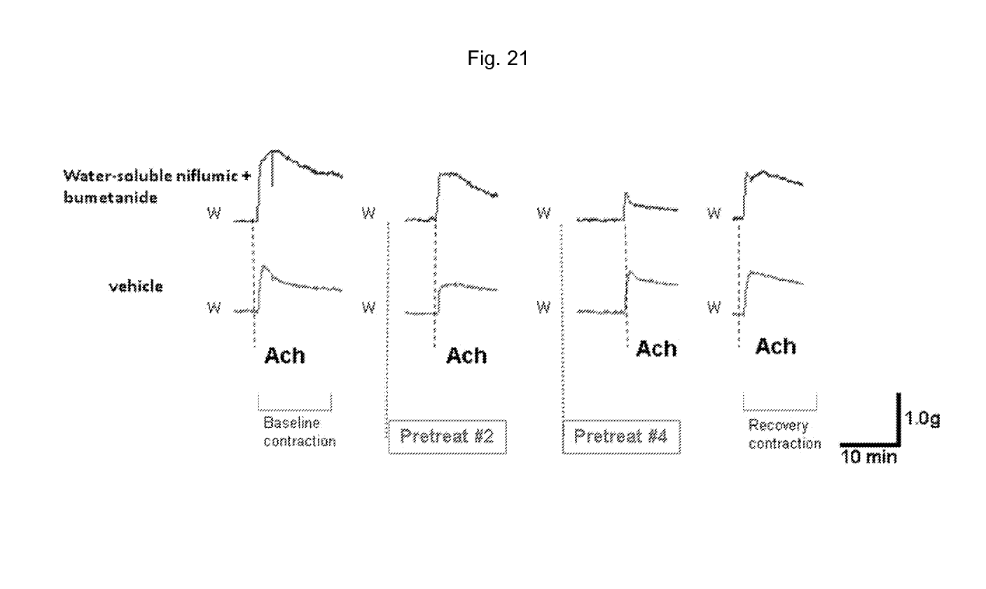

To determine if adding water solubility to NFA allowed it to retain its ability to inhibit acetylcholine-induced contractions in human airway smooth muscle, human airway smooth muscle strips were pretreated with either water-soluble NFA+bumetanide or a vehicle control in organ baths. Contraction magnitude was then recorded. Water-soluble NFA+bumetanide caused a reduction in magnitude of acetylcholine-induced contractions compared to controls. Thus, the water-soluble formulation of niflumic acid also impairs the repetitive acetylcholine-induced contractions in ex vivo human airway smooth muscle in a reversible manner (FIG. 21).

### Example 14

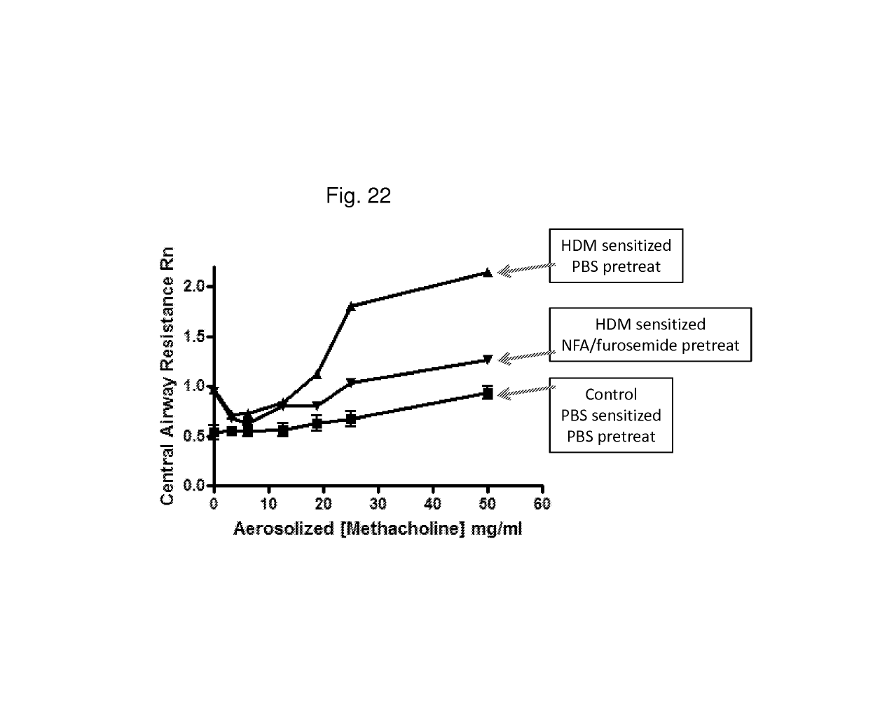

The in vivo feasibility of the drug combination therapy were investigated in a mouse model of asthma. Briefly, these C57 mice were sensitized for 3 weeks to house dust mite antigen by daily nasal inhalation. This is a standard mouse model to make the animals airways hyperreactive and thus is more like a model of human asthma. These animals were then anesthetized, intubated through a tracheostomy and connected to a Flexivent (SciReq®) device that accomplishes 3 tasks: (1) it ventilates the animal, (2) it is capable of delivering an inhaled aerosol of any drug and (3) it measures the airway resistance (a reflection of bronchoconstriction) using a technique referred to as forced oscillation. The exciting finding was that when combination therapy with a CaCC inhibitor (niflumic acid (NFA)) and a NKCC inhibitor (furosemide) was given to the mouse by inhalation (a method of drug delivery most common in human asthma), the mice have less of an airway contractile response to a standard bronchoconstrictive challenge (inhaled methacholine) just 5 minutes after this inhalation, as shown in FIG. 22. This data strongly indicate that a combination drug therapy of chloride channel and chloride transporter modulators works for human asthma.

### Example 15

The following materials and methods were used for Examples 16-22.

### Materials

All materials were obtained from Sigma-Aldrich (St. Louis, Mo.) unless otherwise specified. Tetrodotoxin was purchased from Calbiochem, EMD Biosciences (La Jolla, Calif.). MK571 was purchased from Tocris (Minneapolis, Minn.). Membrane potential dye [fluorescent imaging plate reader (FLIPR) blue reagent] was obtained from Molecular Devices (Sunnyvale, Calif.). A library of chloride channel blockers was a gift from Dr. Landry from the Department of Medicine at Columbia University.

### Synthesis of Compounds 1 and 13

Compounds 1 and 13 were synthesized according to the following general scheme.

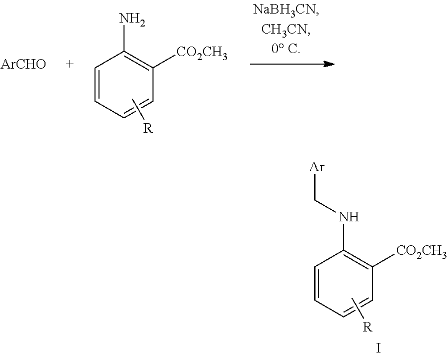

Sodium sulfate (1.5 equivs.) and the corresponding aryl aldehyde (e.g., 4-chlorobenzyl aldehyde for compound 1 or naphthaldehyde for compound 13) (1.3 equivs.) were stirred in anhydrous acetonitrile. Then methyl 2-aminobenzoate (1 equiv.) was added. The reaction mixture was stirred for 30 minutes, after which it was cooled to 0° C., and sodium cyanoborohydride was added. The reaction mixture was stirred overnight. It was subsequently treated with 2 M aqueous HCl followed by 5 M aqueous NaOH. The organic layer was extracted and dried with anhydrous sodium sulfate. The solvent was evaporated in vacuo, yielding compound I.

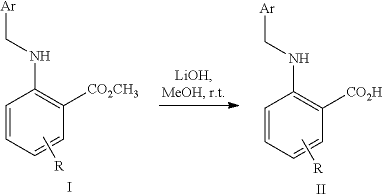

Compound I was hydrolyzed with lithium hydroxide in either methanol or ethanol. The solvent was evaporated in vacuo. Distilled water was added. The organic material is extracted with diethyl ether. The aqueous layer was acidified to a pH of about 3-4 with 1 M aqueous hydrochloric acid. The organic material was extracted with diethyl ether and dried with anhydrous sodium sulfate. The solvent was evaporated in vacuo.

### Guinea Pig Tracheal Rings

Animal protocols were approved by the Columbia University Animal Care and Use Committee. Male Hartley guinea pigs weighing approximately 400 g were anesthetized with intraperitoneal injection of sodium pentobarbital (100 mg/kg). Trachea were removed and transferred to ice cold Krebs-Henseleit (KH) buffer of the following composition (in mM): NaCl 118, KCl 5.6, CaCl₂ 0.5, MgSO₄ 0.24, NaH₂PO₄ 1.3, NaHCO₃ 25, glucose 5.6, pH 7.4. Epithelial-denuded tracheal rings were prepared using a dissecting microscope. Epithelium was removed by gently passing cotton fibers through the lumen of the tracheal rings. The rings were suspended in 4 mL-water jacketed organ baths (37° C.: Radnoti Glass Technology, Monrovia, Calif.) and connected to a Grass FT03 force transducer (Grass Telefactor, West Warwick, R.I.) using silk sutures. The transducers were coupled to the computer with BioPac hardware and data was collected using Acqknowledge 7.3.3 software (BioPac Systems, Goleta, Calif.). KH buffer was bubbled with a gas mixture of 95% oxygen and 5% carbon dioxide and buffer was exchanged every 15 minutes for 1 hour during equilibration of tracheal rings at 1 g resting tension. Unless specified otherwise in the results, all baths received 10⁻⁵M indomethacin to block the effects of endogenous prostanoids, 10⁻⁵ M N-vanillylnonanamide (capsaicin analogue) to activate and subsequently block the effects of non-adrenergic, non-cholinergic nerves, 10⁻⁶ M tetrodotoxin to block neuronal effects, and 10⁻⁵ M of pyrilamine to block effects of endogenous histamine release. Tissue was contracted with either 10 mM tetraethylammonium chloride (TEA), 80 mM potassium gluconate or EC₅₀ dose of acetylcholine (Ach).

### Human Tracheal Strips

Studies using de-identified human tissue were reviewed by the Columbia University Institutional Review Board and deemed not human subjects research. Tissues were obtained from healthy, normal organ donor discarded surgical waste subsequent to lung transplant surgery. Tissues were collected after transplant and placed in DMEM media (GIBCO) and bubbled overnight in 5% carbon dioxide and 95% oxygen at 4° C. The following morning, the smooth muscle tissue was carefully dissected under a dissecting microscope and the epithelium removed. The strips were anchored in the organ bath as described above using KH buffer of the same composition. The strips were allowed to equilibrate for 1 hour with buffer exchanges every 15 minutes and were treated with 10⁻⁶ M tetrodotoxin, 10⁻⁵ M pyrilamine, and 10⁻⁵

M MK571. Strips were contracted with an EC₅₀ dose of acetylcholine (Ach)

### Cultured Human Airway Smooth Muscle Cells

Immortalized airway smooth muscle cell lines modified to stably express human telomerase reverse transcriptase were a kind gift from Dr. William Gerthoffer (University of Southern Alabama, Mobile, Ala.) prepared as described previously (Gosens et al., 2006) and grown in Dulbecco's Modified Eagle's Medium/F12 media (GIBCO, Grand Island, N.Y.), with 10% FBS and antibiotics.

### FLIPR Membrane Potentiometric Dye Assay

Immortalized human airway smooth muscle cells were cultured on black-walled 96 well plates to 100% confluence, and were washed four times with warmed (37° C.) assay buffer of the following composition (in mM): NaCl 140, KCl 4.7, CaCl₂ 2.5, MgCl₂ 1.2, HEPES 11, D-glucose 10. A stock solution (100% dye) of FLIPR blue dye was prepared by reconstitution of 1 vial (125 mg) with 100 ml assay buffer. A 50% working stock was prepared by further diluting the reconstituted blue dye 1:1 with assay buffer and was used to load cells (90 μl/well) over 45 min at 37° C. in a humidified cell culture incubator (95% air/5% CO₂). All reagents were dissolved in assay buffer. Fluorescence was measured with a Flex Station 3 (Molecular Devices) using an excitation wavelength of 530 nm, emission wavelength of 565 nm and a cutoff filter of 550 nm. Baseline fluorescence was measured every 2 seconds for 1 minute then for 2 minutes after injection of vehicle or compound of interest as indicated (using the Flexstation's 3 integrated injection capabilities).

### Patch-Clamp Analysis

Cells were plated on poly-L-Lysine 12 mm coverslips (Becton, Dickinson and Company, Park Bedford, Mass.) coated with 0.5 mg/ml collagen Type I (Sigma). The coverslips were transferred to 0.5 ml chambers on the stage of an inverted microscope (Nikon, Japan). Membrane currents were recorded using tight-seal conventional whole-cell configuration. The extracellular solution contained (in mM) 130 NaCl, 5.5 TEA-Cl, 2.2 CaCl₂, 1 MgCl₂, 10 HEPES, 10 Glucose, pH adjusted to 7.4 with NaOH. The pipette solution contained (in mM): 75 CsCl, 64 Cs-gluconate, 1 MgCl₂, 10 HEPES, 3 Na₂ATP, pH adjusted to pH 7.3 with CsOH (Bao et al., 2008). Cesium and TEA were used in intracellular and extracellular solutions, respectively, to block K⁺ channels to allow for the study of chloride channel activity. Whole-cell currents were recorded using Axopatch 200B and digitized using 1322A. Patch pipettes had resistances of 3-6 MO. All recordings were performed at room temperature. Events were counted as spontaneous transient inward currents (STICs) if their amplitude exceeded twofold of the baseline as detected with pCLAMP10 and analysis by Origin 8 software.

### Statistics

Data were analyzed using one-way ANOVA with repeated measures. The Bonferroni correction was applied for multiple comparisons. Statistical significance was established at P<0.05, and all values are expressed as means±SE.

### Example 16

This example shows that chloride channel blockers relax a TEA-induced contraction.

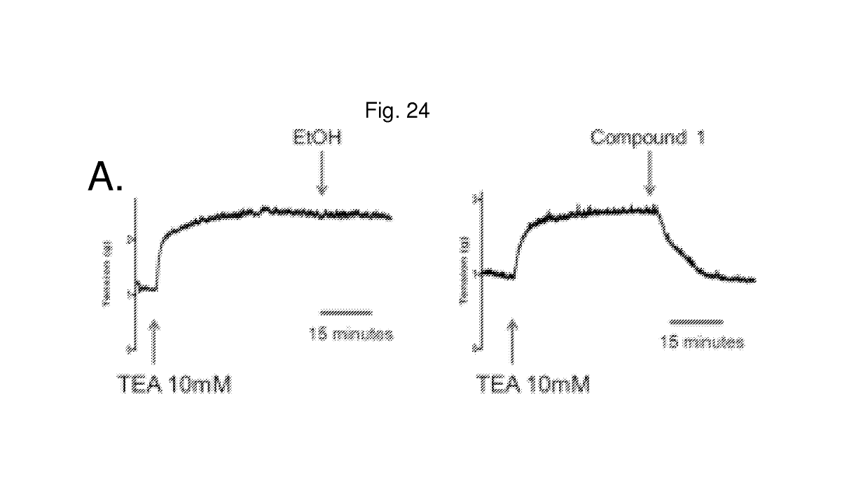

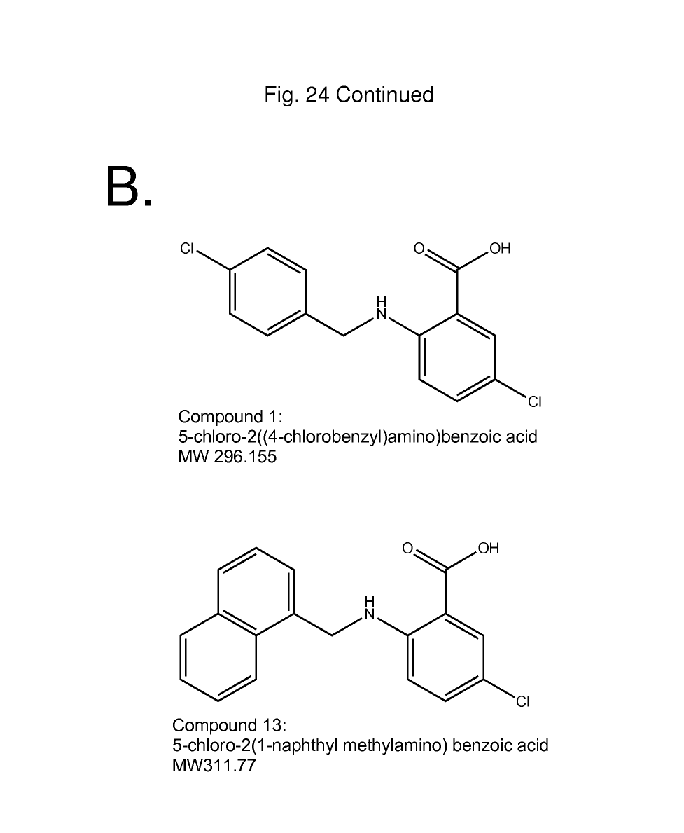

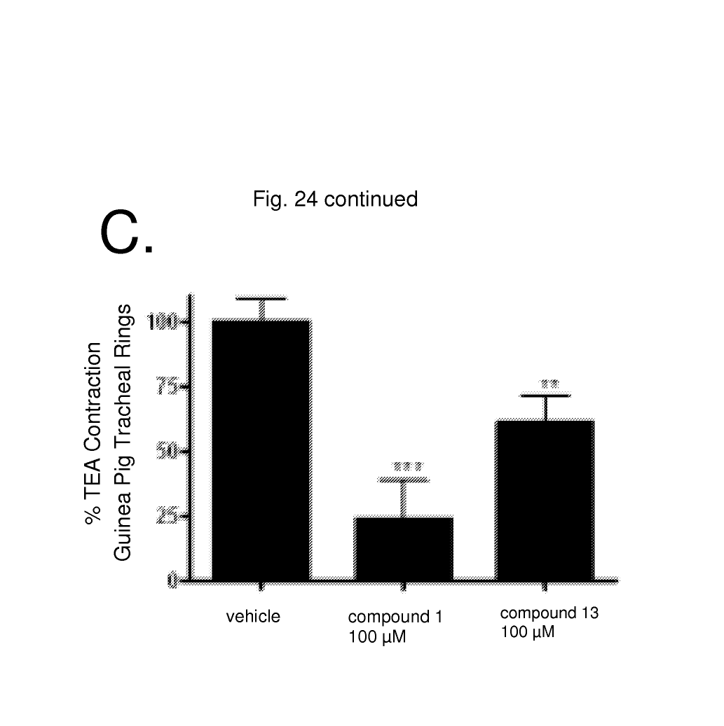

Chloride channel blockers were initially tested for their ability to reverse a depolarized-induced contraction in guinea pig airway smooth muscle, as chloride flux has been shown to be important in depolarization (Janssen et al., 1995). All twenty compounds of the chloride channel blocker library were screened against a contraction induced by tetraethylammonium chloride (TEA), a potassium channel blocker that causes a depolarization of airway smooth muscle by blocking potassium efflux. Anthranilic acid derivatives such as compounds 1 (5-chloro-2-((4-chlorobenzyl) amino)benzoic acid) and 13 (5-chloro-2-(1-naphthyl methylamino)benzoic acid) (see structures in FIG. 24B) were found to relax a TEA-induced contraction in guinea pig tracheal rings (FIG. 24A), while derivatives of indanyloxyacetic acid failed to relax (not shown). Of all 20 compounds, compounds 1 and 13 were found to most potently relax and were therefore chosen for further study. Compound 1 (100 μM) relaxed 76.2% and compound 13 (100 μM) relaxed 39.0% of a TEA-induced contraction at 15 minutes after drug addition (23.8±14.9 (p<0.001, n=9), 61.0±10.1 (p<0.01, n=9), respectively, percent of the TEA-induced contraction remaining at 15 minutes) (FIG. 24C).

### Example 17

This Example shows that chloride channel blockers relax a K-gluconate contraction.

Many chloride channel blockers are known to also stimulate potassium channels (Greenwood et al., 2007). Thus, we performed additional studies in the presence of a large extracellular concentration of potassium to ensure that our findings of compound 1 or 13 inhibiting a TEA-induced depolarizing contraction were not due to these compounds opening a potassium channel. Relaxation of airway smooth muscle by chloride channel blockers, in the presence of a large potassium gradient (80 mM K-gluconate) unfavorable for K⁺ efflux, would suggest that compounds 1 and 13 were not inducing relaxation of depolarization-induced contractions by opening of a potassium channel.

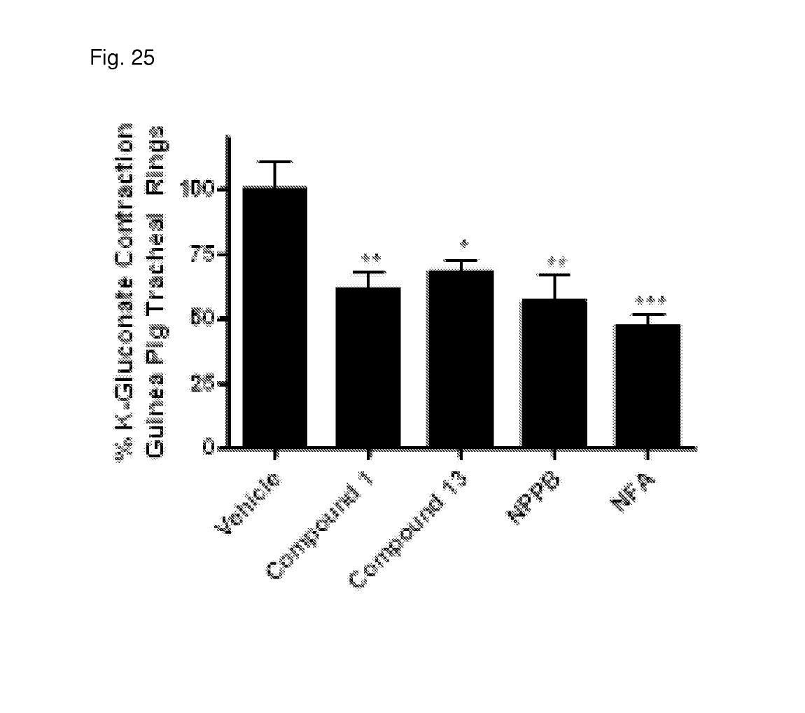

Both compounds 1 and 13 (at 100 μM) were able to relax a 80 mM potassium gluconate-induced contraction (61.46%±6.3 (p<0.01, n=6), 68.17%±4.9 (p<0.05, n=6), respectively, percent of potassium gluconate-induced contraction at 30 minutes) (FIG. 25). Classic chloride channel blockers 5-nitro-2-(3-phenylpropylamino)benzoic acid (NPPB) (100 μM) and niflumic acid (NFA) (100 μM) were used as positive controls and also showed significant relaxation (56.71%±10.6 (p<0.01, n=4), 47.36%±4.6 (p<0.01, n=4), respectively, percent of potassium gluconate-induced contraction at 30 minutes). The ability of compounds 1 and 13 to functionally mimic classic chloride channel blockers is consistent with the conclusion that compounds 1 and 13 are blockers of chloride channels in airway smooth muscle.

### Example 18

This Example shows that a combination of chloride channel blockers and NKCC blockade attenuates the initiation of an acetylcholine-induced contraction.

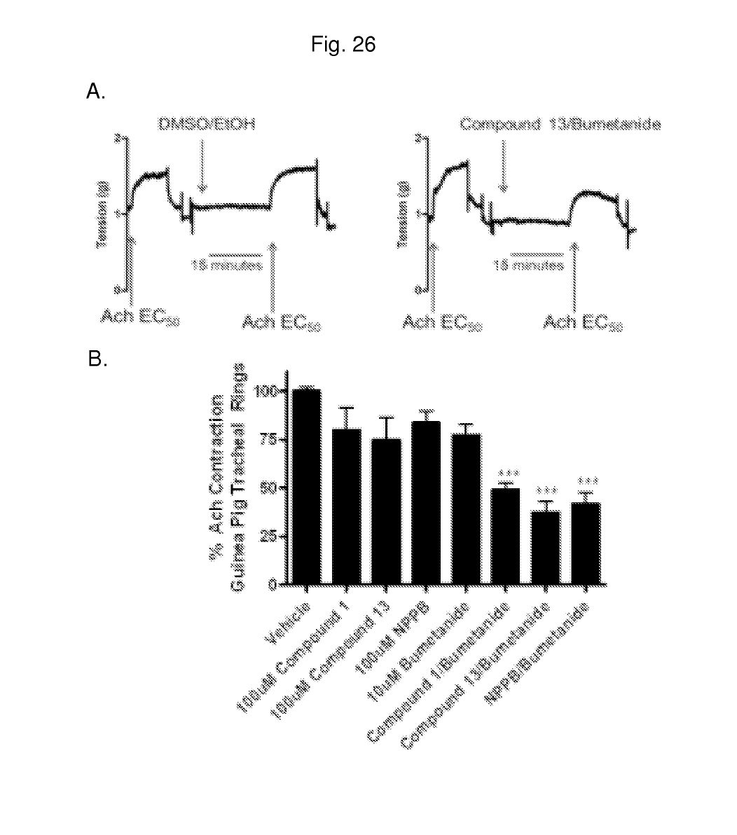

Compounds 1 and 13 were tested for their abilities to attenuate the initiation phase of an acetylcholine contraction, as acetylcholine is a natural bronchoconstrictor and prevention of contraction would have clinical utility. While pretreatment with chloride channel blockers or the sodium potassium chloride cotransporter (NKCC) blocker bumetanide separately failed to significantly attenuate a contraction, a combination of chloride channel blockers and bumetanide significantly attenuated an acetylcholine contraction compared to vehicle controls (FIG. 26A). With pretreatment with both compound 1 (100 μM) and bumetanide (10 μM) for 15 minutes, an EC₅₀ acetylcholine contraction was only 48.9%±3.6 of vehicle (0.1% ethanol and 0.1% DMSO) control contractions (p<0.001, n=5), while combination of compound 13 (100 μM) with bumetanide (10 μM) or NPPB (100 μM) with bumetanide (10 μM) showed similar levels of attenuation (37.5%±5.8 and 41.5%±6.3, respectively (p<0.001, n=5)). Compounds alone did not demonstrate a statistically significant attenuation of contraction (FIG. 26B). Therefore, in order to prevent or attenuate the initiation phase of a contraction by blockade of chloride flux, a combination of compound 1 or 13 with an NKCC blocker was required.

### Example 19

This example shows that chloride channel blockers relax an acetylcholine contraction in guinea pig tracheal rings and human airway smooth muscle.

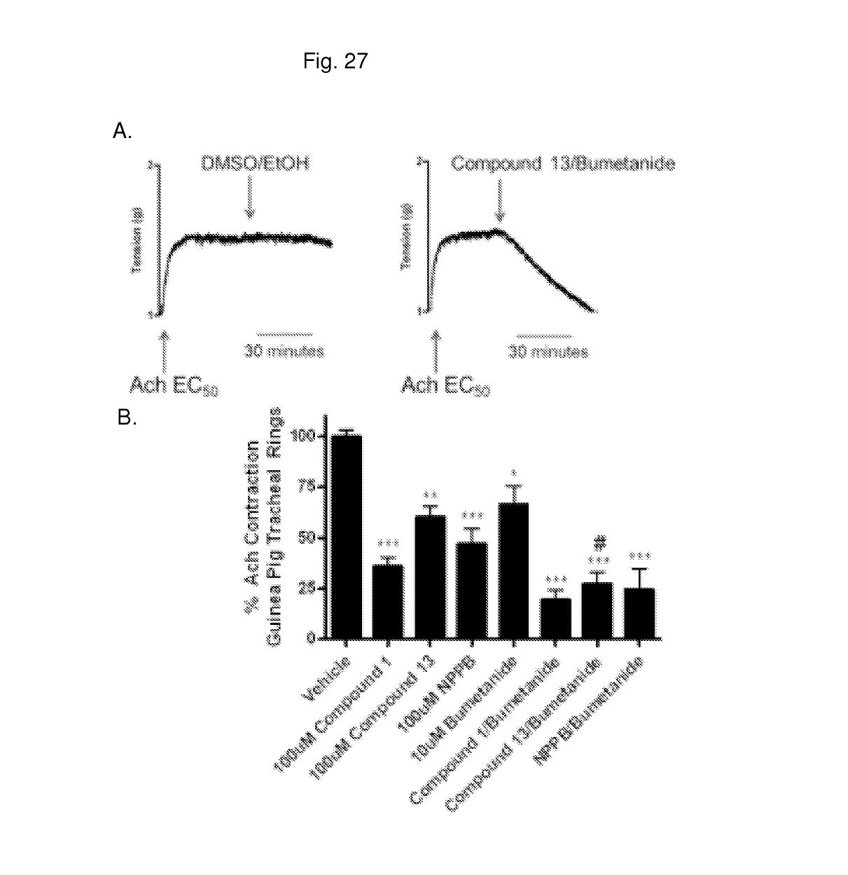

Chloride channel blockers were then tested against the plateau phase of an established acetylcholine contraction. Guinea pig tracheal rings were pre-contracted with an EC₅₀ dose of acetylcholine and then chloride channel blockers were added to the organ bath in order to investigate whether the chloride channel blockers had an effect on the maintenance phase of contraction, in addition to the initiation of contraction as demonstrated above. Treatment with chloride channel blockers alone, bumetanide alone or the combination all significantly relaxed an acetylcholine contraction (FIGS. 27A and 27B). Relaxation induced by compound 13 (100 μM) (60.1%±5.7, p<0.01, n=6) but not compound 1 (100 μM) was further potentiated when combined with bumetanide (27.3%±5.6, p<0.05 compared to compound 13 alone, n=6) (FIG. 27B). Therefore, in order to relax an established acetylcholine contraction, chloride channel blockade or NKCC blockade alone was sufficient.

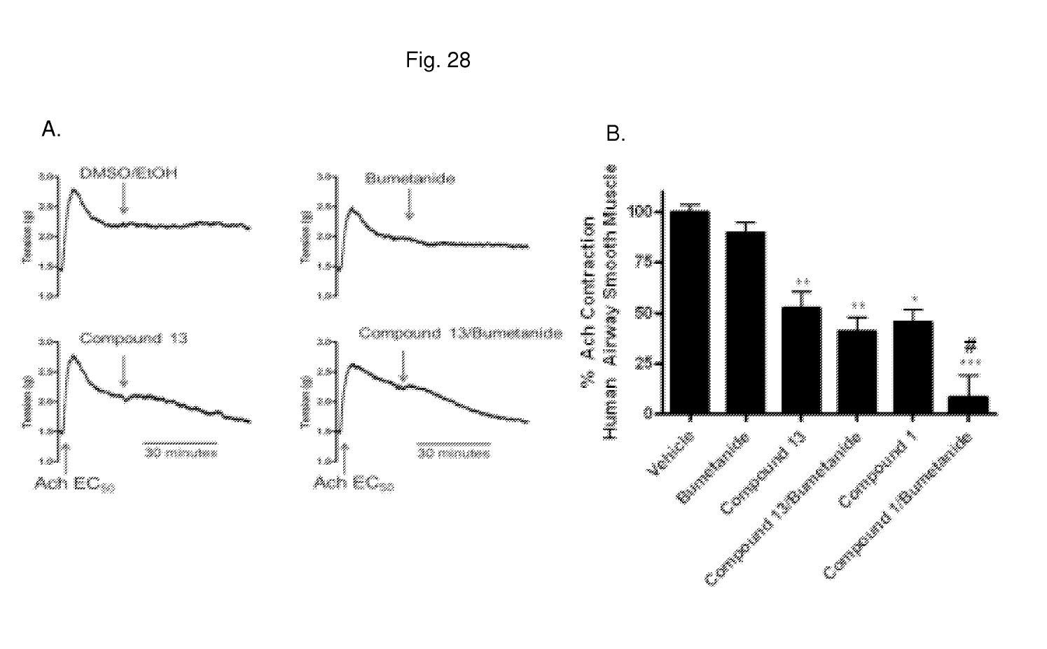

This experiment was then repeated with human airway smooth muscle to investigate clinical relevance as a model of bronchospasm in humans. In human airway smooth muscle, vehicle (0.1% ethanol, or 0.1% DMSO) or bumetanide treatment failed to relax an acetylcholine contraction, while compound 1 (100 μM) and 13 (100 μM) alone (45.4%±6.3 (p<0.05, n=9), 52.5%±8.1 (p<0.01, n=9), respectively) and in combination with bumetanide (8.2%±11.1 (p<0.001, n=13), 41.1%±6.7 (p<0.001, n=13), respectively) relaxed an acetylcholine contraction (FIGS. 28A and 28B). In the human model, only relaxation by compound 1 was significantly increased when combined with bumetanide (p<0.05 compared to compound 1 alone). Bumetanide alone did not demonstrate significant relaxation, and compound 1 relaxation instead of 13 (as seen in guinea pig organ bath data above) was significantly increased in combination with bumetanide, suggesting a potential species difference. However, in both species chloride channel blockers significantly relaxed an acetylcholine contraction.

### Example 20

This Example shows that a combination of chloride channel blockers and bumetanide potentiate β agonist relaxation by isoproterenol in guinea pig tracheal rings.

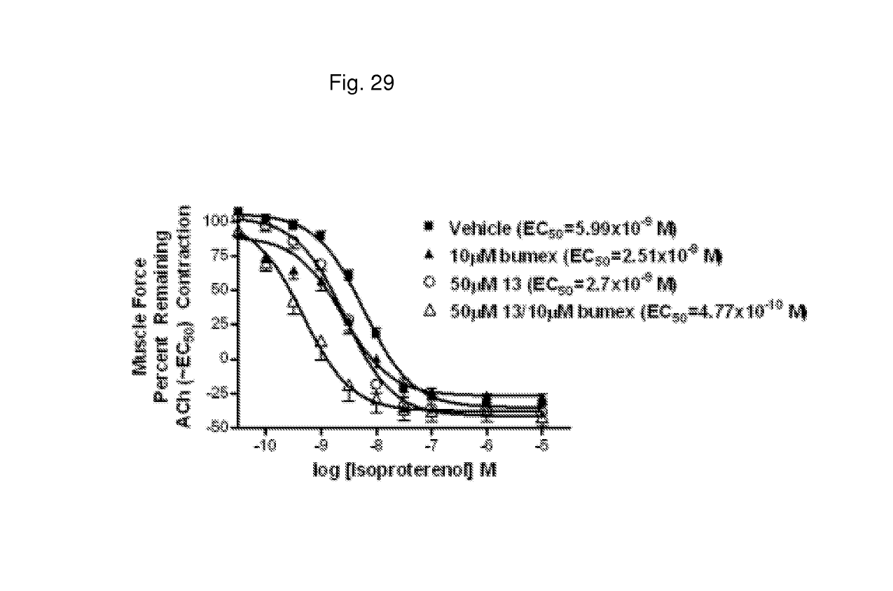

β-agonists remain the first line of treatment for asthma or acute bronchospasm, although severe asthmatics have been observed to become refractory to these agents. Therefore, we tested whether the chloride channel blockers or modulation of chloride flux could be used in combination with β-agonists to potentiate their effects. The EC₅₀ of isoproterenol-induced relaxation was shifted from 5.99×10⁻⁹M to 4.77×10⁻¹⁰ M (p<0.01, n=6) with a combination of 50 μM compound 13 and 10 μM bumex (bumetanide), which is a 12.6 fold or greater than one log shift of the EC₅₀ of isoproterenol (FIG. 29). 10 μM bumetanide or 50 μM compound 13 by themselves did not significantly shift relaxation by isoproterenol (2.51×10⁻⁹, 2.7×10⁻⁹ M, respectively (p>0.05)), with a 2.4 fold shift and 2.2 fold shift, respectively. Therefore, a combination of treatment with compound 13 and bumetanide potentiated the relaxation effect of isoproterenol.

### Example 21

This Example shows that chloride channel blockers hyperpolarize human airway smooth muscle cells.

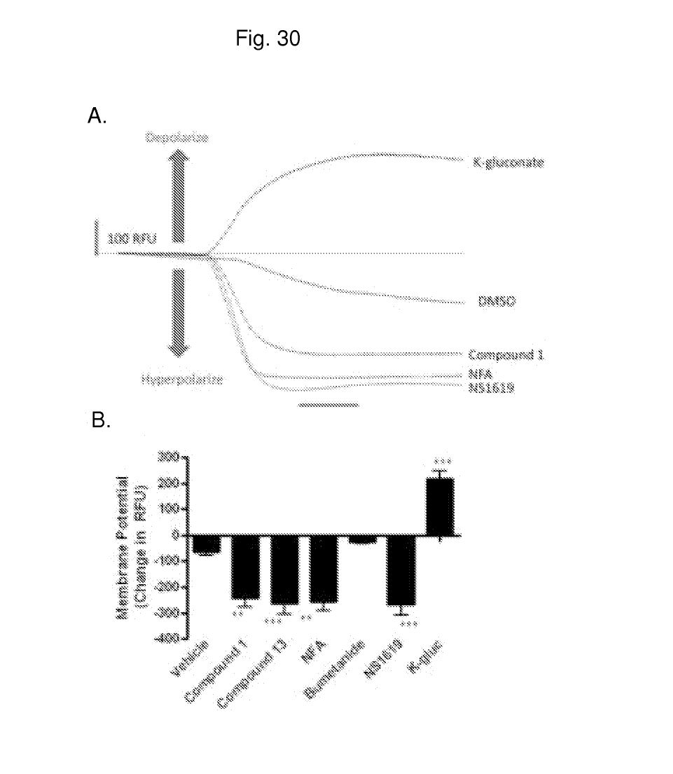

Human airway smooth muscle cells were incubated with FLIPR membrane potentiometric dye to assess if the chloride channel blockers changed membrane potential at the cellular level. FLIPR fluorescence will increase with depolarization, and decrease with hyperpolarization. 100 μM compound 1 (−240.3+/−35 RFU, p<0.01, n=12), compound 13 (−264.2−/−40 RFU, p<0.001, n=12) and niflumic acid (−254.7+/−37 RFU, p<0.01, n=12) all caused a hyperpolarization, while 10 μM bumetanide alone did not cause a significant change (FIG. 30B). Therefore, chloride channel blockade, but not NKCC blockade, hyperpolarized human airway smooth muscle cells. Chloride channel blockers hyperpolarized membrane potential in human airway smooth muscle cells, which is consistent with this being a cellular mechanism by which relaxation was demonstrated in organ bath studies.

### Example 22

This example shows that chloride channel blockers inhibit spontaneous transient inward current (STIC) activity in human airway smooth muscle cells.

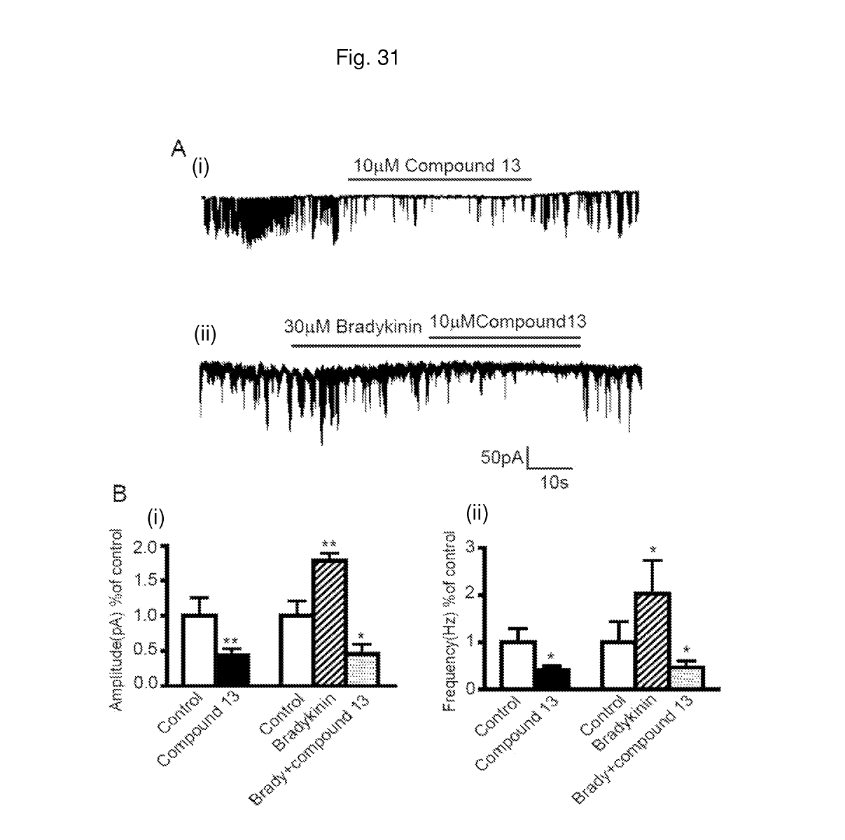

Many different types of chloride channels exist on human airway smooth muscle cells, but one of the most important types of chloride channels involved in depolarization and airway hyperresponsiveness is the calcium-activated chloride channel (CaCC) (Huang et al., 2012; Zhang et al., 2013). Calcium sparks activate CaCCs, leading to chloride efflux and depolarization of the cell, which in turn leads to further contraction with the opening of voltage-dependent calcium channels (Janssen et al., 1995). Therefore, compound 13 was tested for its specific ability to block this channel. The activity of this channel can be measured via electrophysiology by measuring STICs (Janssen et al., 1994). Under whole-cell voltage-clamp configuration, STICS were identified from human airway smooth muscle cells using a holding potential of −60 mV (FIG. 31A(i)).

The average peak amplitude and frequency of the current under control conditions was 124.2±2 pA and 5.61, 2.7 Hz, respectively (calculated 599 events from 14 cells). Compound 13 decreased both the amplitude (44% of control, p<0.01) and frequency of STICs (40% of control, p<0.05) under basal conditions (n=9) (FIG. 31B(i) and (ii)). Elevations of intracellular calcium will increase STIC activity and thus bradykinin was used in the present study due to its well established ability to increase intracellular calcium in these cells. Bradykinin stimulation effect was inhibited by 10 μM compound 13 (FIG. 31A(ii)). While bradykinin increased the amplitude (179%) and frequency (203%) when compared to control, in the presence of 10 μM compound 13 and bradykinin the amplitude (46%) and frequency (40%) decreased to below control values (n=5, p<0.05) (FIG. 31B(ii)). This study suggests that compound 13 is a potential Ca²⁺-activated chloride channel blocker, which may play a modulate role on bradykinin induced depolarization and muscle contraction in human airway smooth muscle.

In sum, we have shown that chloride channels and transporters are important in both the initiation and maintenance of airway smooth muscle contraction. A library of chloride channel blockers were first screened for their ability to relax a contraction induced by TEA depolarization. Two of these compounds that demonstrated potent relaxation were then evaluated for their ability to either prevent the initiation of a contraction induced by an endogenous Gq-coupled bronchoconstrictor, acetylcholine, or to relax an established acetylcholine contraction. Furthermore, the effect of blockade of the chloride transporter, NKCC1, either alone or in combination with chloride channel blockade, against the initiation or maintenance of an acetylcholine contraction was measured. Chloride channel blockers were more effective in relaxing a pre-established acetylcholine contraction (chloride channel blockers alone, or NKCC blockade alone both effectively relaxed a contraction) than in inhibiting the initiation of a contraction (which required co-blockade with NKCC inhibitors to significantly inhibit contraction).

Not wishing to be bound by a particular theory, but one possible explanation for this difference is the subcellular localization of chloride channels in airway smooth muscle cells and the effect of chloride channel blockade on calcium signaling at the plasma versus sarcoplasmic reticulum membranes. Janssen et al previously showed in bovine airway smooth muscle that treatment with the chloride channel blocker NPPB reduced the magnitude of successive acetylcholine contractions, while another chloride channel blocker niflumic acid (NFA) did not (Hirota et al., 2006). They argued that NPPB had a higher selectively for chloride channels on the sarcoplasmic reticulum (SR) than NFA (Pollock et al., 1998), and that NPPB may also be inhibiting chloride flux at the SR with resultant run down in the SR calcium pools after repetitive contractile challenges. Although chloride efflux through calcium activated chloride channels on the plasma membrane has been shown to be important in initial depolarization (Janssen et al., 1995), which subsequently induces voltage-gated calcium entry into the cell and calcium induced calcium release from the SR, Janssen, et al. hypothesized that chloride flux on the SR membrane dictates SR calcium release and refilling by neutralizing the charge balance across the SR membrane. Thus, by blocking chloride channels on the SR, calcium may not be able to leave the SR to maintain a contraction. Our compounds were compared against NPPB and were found to have a similar potency and relaxation profile. Therefore, they may also play an important role in calcium signaling regulated at the SR in airway smooth muscle. Chloride channel blockers may be more effective in blocking the maintenance of a contraction as it requires constant refilling of calcium and release from the SR which may be inhibited by blocking chloride flux.

Previous studies have shown the importance of calcium activated chloride channel blockade in airway smooth muscle. Huang et al. (2012) showed that pretreatment with benzbromarone, a CaCC blocker, can partially attenuate a methacholine contraction in human airway smooth muscle isolated from a single individual. Zhang et al. (2013) have used an ovalbumin-sensitized mouse model to show that intravenous pretreatment with niflumic acid or benzbromarone prevented airway hyperresponsiveness following inhaled methacholine. These studies differ from the present study in that pretreatment with our chloride channel blockers was insufficient to attenuate an acetylcholine-induced contraction unless combined with an NKCC blocker (i.e. bumetanide). There are several variations in study design that may account for these differences. The studies conducted in isolated human bronchial rings were from a single individual and used 10 μM benzobromarone, a compound believed to act by inhibiting TMEM16 or anoctamin channels, the recently identified family of calcium-activated chloride channels. The specificity and potency of compound 1 and 13 used in the present study for the TMEM16 family of channels is currently unknown. Zhang et. al, used an in vivo sensitized mouse model while our studies were performed in non-sensitized ex vivo airway smooth muscle from guinea pigs and humans. Thus, our results may differ from these mouse studies due to species differences, the mode of delivery or differences in potency and specificity between the chloride channel blockers used, or the study of ex vivo airway smooth muscle contraction versus in vivo lung resistance. Importantly, we also investigated the ability of our compounds to relax an existing pre-established acetylcholine contraction, which may more closely mimic a constricted airway during an asthmatic exacerbation. Thus, our study is the first to show relaxation of a pre-established acetylcholine contraction in ex vivo human and guinea pig airway smooth muscle by chloride channel blockade.

NKCC was previously shown to be present in human (Iwamoto et al., 2003) and guinea pig (Rhoden et al., 1995) airway smooth muscle. As seen in our current study, NKCC inhibitors such as furosemide have been shown to play a role in airway smooth muscle relaxation in guinea pig airway smooth muscle (Stevens et al., 1992). In these studies, more significant relaxation was seen in fetal and newborn airway tissue than adult guinea pig tissue. Clinically, furosemide has effectiveness in reducing airway resistance in premature human infants (Rastogi et al., 1994; Kao et al., 1983) and in selective challenges in adults (Bianco et al., 1988; Grubbe et al., 1990; Prandota et al., 2002), but is not universally effective in all studies.

We have observed a significant relaxation in the adult guinea pig, but did not observe any significant relaxation with bumetanide treatment alone in adult human airway smooth muscle. However, bumetanide did increase relaxation in combination with compound 1 when compared with compound 1 alone in adult human airway smooth muscle. Therefore, although NKCC blockade alone does not cause significant relaxation in adult human airway smooth muscle, combination with another chloride channel blocker can be augmented with NKCC blockade. Interestingly, compound 13 relaxation was augmented in guinea pig with bumetanide, but compound 1 was augmented by bumetanide in human airway smooth muscle, suggesting a potential species or age difference in compound 1 versus compound 13's effects. Our studies are the first to use NKCC blockade in combination with CaCC blockade to prevent contraction, or to augment relaxation of a preexisting contraction.

We have also shown that a combination of CaCC and NKCC blockade was able to potentiate relaxation of the beta agonist isoproterenol. As chloride channel blockers could be clinically indicated in combination with first line agents such as beta agonists and severe asthmatics can become resistant, potentiation of beta agonist relaxation can be of great clinical utility. These studies also strongly suggest that chloride channel blockers show promise as a bronchodilator, as potentiation would imply that chloride channel blockers work via a different mechanism or signaling pathway than beta agonists.

In isolated human airway smooth muscle cells our novel chloride channel blockers hyperpolarized the plasma cell membrane which would favor relaxation of airway smooth muscle. Furthermore, we utilized spontaneous transient inward currents (STICs), an electrophysiological event resulting from calcium activation of calcium-activated chloride channels (CaCCs) in airway smooth muscle (Janssen et al., 1994) to implicate this specific family of chloride channels in our compounds' effects. The reversible blockade of STICs by compound 13 would imply inhibition of plasma membrane CaCCs by our novel chloride channel blockers.

In conclusion, inter alia, we have identified two chloride channel blockers that relax an acetylcholine contraction, and in combination with NKCC blockade prevent initiation of an acetylcholine contraction and potentiate beta agonist relaxation. These two novel chloride channel blockers also hyperpolarize airway smooth muscle cells, and compound 13 inhibits calcium activated chloride channel activity. Modulation of chloride flux is a promising new treatment of asthma and bronchospasm.

### DOCUMENTS

Bao, R., et al., A close association of RyRs with highly dense clusters of Ca2+-activated Cl— channels underlies the activation of STICs by Ca2+ sparks in mouse airway smooth muscle. J Gen Physiol, 2008. 132(1): p. 145-60.
Ben-Chaim Y, Chanda B, Dascal N, Bezanilla F, Parnas I, Parnas H: Movement of ‘gating charge’ is coupled to ligand binding in a G-protein-coupled receptor. Nature 2006; 444: 106-9
Bianco, S., et al., Prevention of exercise-induced bronchoconstriction by inhaled frusemide. Lancet, 1988. 2(8605): p. 252-5.
Billups D, Billups B, Challiss R A, Nahorski S R: Modulation of Gq-protein-coupled inositol trisphosphate and Ca2+ signaling by the membrane potential. Journal of Neuroscience 2006; 26: 9983-95
Corteling R L, Li S, Giddings J, Westwick J, Poll C, Hall I P: Expression of transient receptor potential C6 and related transient receptor potential family members in human airway smooth muscle and lung tissue. American Journal of Respiratory Cell and Molecular Biology 2004; 30: 145-54
Deshpande D A, Wang W C, Mcllmoyle E L, Robinett K S, Schillinger R M, An S S, Sham J S, Liggett S B: Bitter taste receptors on airway smooth muscle bronchodilate by localized calcium signaling and reverse obstruction. Nat. Med. 2010; 16: 1299-304
Duan Q L, Tantisira K G: Pharmacogenetics of asthma therapy. Curr. Pharm. Des 2009; 15: 3742-53
Engelhardt B, Elliott S, Hazinski T A. Short- And Long-Term Effects Of Furosemide On Lung Function In Infants With Bronchopulmonary Dysplasia. J. Pediatr. 1986; 109(6):1034-9.
Gallos G, Gleason N R, Virag L, Zhang Y, Mizuta K, Whittington R A, Emala C W: Endogenous gamma-aminobutyric acid modulates tonic guinea pig airway tone and propofol-induced airway smooth muscle relaxation. Anesthesiology 2009; 110: 748-58
Gallos G, Gleason N R, Zhang Y, Pak S W, Sonett J R, Yang J, Emala C W: Activation of endogenous GABAA channels on airway smooth muscle potentiates isoproterenol mediated relaxation. Am. J. Physiol Lung Cell Mol. Physiol 2008
Gallos G, Yim P, Chang S, Zhang Y, Xu D, Cook J M, Gerthoffer W T, Emala C W, Sr.: Targeting the restricted alpha subunit repertoire of airway smooth muscle GABAA receptors augments airway smooth muscle relaxation. Am. JPhysiol Lung Cell Mol. Physiol 2011.
Gallos, G., et al., Targeting the restricted alpha-subunit repertoire of airway smooth muscle GABAA receptors augments airway smooth muscle relaxation. Am J Physiol Lung Cell Mol Physiol, 2012. 302(2): p. L248-56.
Gleason N R, Gallos G, Zhang Y, Emala C W: Propofol preferentially relaxes neurokinin receptor-2-induced airway smooth muscle contraction in guinea pig trachea. Anesthesiology 2010; 112: 1335-44
Gleason N R, Gallos G, Zhang Y, Emala C W: The GABAA agonist muscimol attenuates induced airway constriction in guinea pigs in vivo. J. Appl. Physiol 2009; 106: 1257-63
Gosens, R., et al., Role of caveolin-1 in p42/p44 MAP kinase activation and proliferation of human airway smooth muscle. Am J Physiol Lung Cell Mol Physiol, 2006. 291(3): p. L523-34.
Greenwood, I. A. and N. Leblanc, Overlapping pharmacology of Ca2+-activated Cl— and K+ channels. Trends Pharmacol Sci, 2007. 28(1): p. 1-5.
Gromada J, Dissing S: Membrane potential and cytosolic free calcium levels modulate acetylcholine-induced inositol phosphate production in insulin-secreting BTC3 cells. Biochimica et Biophysica Acta 1996; 1310: 145-8
Grubbe, R. E., et al., Effect of inhaled furosemide on the bronchial response to methacholine and cold-air hyperventilation challenges. J Allergy Clin Immunol, 1990. 85(5): p. 881-4.
Gupta S, Meena H S, Chopra R, Bidani R: Effect of aerosolised verapamil and nifedipine in bronchial asthma. J. Assoc. Physicians India 1993; 41: 425-7
Hirota, S., et al., Intracellular Cl— fluxes play a novel role in Ca2+ handling in airway smooth muscle. Am J Physiol Lung Cell Mol Physiol, 2006. 290(6): p. L1146-53.
Huang, F., et al., Calcium-activated chloride channel TMEM16A modulates mucin secretion and airway smooth muscle contraction. Proc Natl Acad Sci USA, 2012. 109(40): p. 16354-9.
Iwamoto, L. M., D. M. Gries, and K. T. Nakamura, Loop diuretics and in vitro relaxation of human fetal and newborn mouse airways. Pediatr Res, 2001. 50(2): p. 273-6.
Iwamoto, L. M., K. T. Nakamura, and R. K. Wada, Immunolocalization of a Na—K-2Cl cotransporter in human tracheobronchial smooth muscle. J Appl Physiol, 2003. 94(4): p. 1596-601.
Janssen L J, Sims S M: Ca²⁺-dependent Cl⁻ current in canine tracheal smooth muscle cells. Am. J. Physiol 1995; 269: C163-C169
Janssen L J, Tazzeo T, Zuo J, Pertens E, Keshavjee S: KCl evokes contraction of airway smooth muscle via activation of RhoA and Rho-kinase. Am. J. Physiol Lung Cell Mol. Physiol 2004; 287: L852-L858
Janssen L J: Ionic mechanisms and Ca²⁺ regulation in airway smooth muscle contraction: do the data contradict dogma? Am. J. Physiol Lung Cell Mol. Physiol 2002; 282: L1161-L1178
Janssen L J: T-type and L-type Ca²⁺ currents in canine bronchial smooth muscle: characterization and physiological roles. Am. J. Physiol 1997; 272: C1757-C1765
Janssen, L. J. and K. Killian, Airway smooth muscle as a target of asthma therapy: history and new directions. Respir Res, 2006. 7: p. 123.
Janssen, L. J. and S. M. Sims, Ca(2+)-dependent Cl— current in canine tracheal smooth muscle cells. Am J Physiol, 1995. 269(1 Pt 1): p. C163-9.
Janssen, L. J. and S. M. Sims, Spontaneous transient inward currents and rhythmicity in canine and guinea-pig tracheal smooth muscle cells. Pflugers Arch, 1994. 427(5-6): p. 473-80.
Jia Y, Wang X, Varty L, Rizzo C A, Yang R, Correll C C, Phelps P T, Egan R W, Hey J A: Functional TRPV4 channels are expressed in human airway smooth muscle cells. Am. J. Physiol Lung Cell Mol. Physiol 2004; 287: L272-L278
Jooste E, Zhang Y, Emala C W: Neuromuscular blocking agents' differential bronchoconstrictive potential in Guinea pig airways. Anesthesiology 2007; 106: 763-72
Kao, L. C., et al., Furosemide acutely decreases airways resistance in chronic bronchopulmonary dysplasia. J Pediatr, 1983. 103(4): p. 624-9.
Kazani S, Ware J H, Drazen J M, Taylor D R, Sears M R: The safety of long-acting beta-agonists: more evidence is needed. Respirology. 2010; 15: 881-5
Kotlikoff M I, Kume H, Tomasic M: Muscarinic regulation of membrane ion channels in airway smooth muscle cells. Biochemical Pharmacology 1992; 43: 5-10
   Landry, D. W., et al., Epithelial chloride channel. Development of inhibitory ligands. J Gen Physiol, 1987. 90(6): p. 779-98.
Liccardi, G., et al., Bronchial asthma. Curr Opin Anaesthesiol, 2012. 25(1): p. 30-7.
Liu C, Zuo J, Janssen L J: Regulation of airway smooth muscle RhoA/ROCK activities by cholinergic and bronchodilator stimuli. European Respiratory Journal 2006; 28: 703-11
Liu C, Zuo J, Pertens E, Helli P B, Janssen L J: Regulation of Rho/ROCK signaling in airway smooth muscle by membrane potential and [Ca²⁺]i. Am. J. Physiol Lung Cell Mol. Physiol 2005; 289: L574-L582
Masoli, M., et al., The global burden of asthma: executive summary of the GINA Dissemination Committee report. Allergy, 2004. 59(5): p. 469-78.
Mizuta K, Xu D, Pan Y, Comas G, Sonett J R, Zhang Y, Panettieri R A, Jr., Yang J, Emala C W, Sr.: GABAA receptors are expressed and facilitate relaxation in airway smooth muscle. Am. J. Physiol Lung Cell Mol. Physiol 2008; 294: L1206-L1216
Perez-Zoghbi J F, Sanderson M J: Endothelin-induced contraction of bronchiole and pulmonary arteriole smooth muscle cells is regulated by intracellular Ca²⁺ oscillations and Ca²⁺ sensitization. Am. J. Physiol Lung Cell Mol. Physiol. 2007; 293: L1000-L1011
Pollock, N. S., M. E. Kargacin, and G. J. Kargacin, Chloride channel blockers inhibit Ca2+ uptake by the smooth muscle sarcoplasmic reticulum. Biophys J, 1998. 75(4): p. 1759-66.
Prabhu V G, Keszler M, Dhanireddy R. Dose-Dependent Evaluation Of The Effects Of Nebulized Furosemide On Pulmonary Function In Ventilated Preterm Infants. J. Perinatol. 1998; 18(5):357-60.
Prabhu V G, Keszler M, Dhanireddy R. Pulmonary Function Changes After Nebulised And Intravenous Frusemide In Ventilated Premature Infants. Arch Dis Child Fetal Neonatal Ed. 1997; 77(1):F32-5.
Prandota, J., Furosemide: progress in understanding its diuretic, anti-inflammatory, and bronchodilating mechanism of action, and use in the treatment of respiratory tract diseases. Am J Ther, 2002. 9(4): p. 317-28.
Rastogi, A., et al., Nebulized furosemide in infants with bronchopulmonary dysplasia. J Pediatr, 1994. 125(6 Pt 1): p. 976-9.
Rhoden, K. J. and J. S. Douglas, Evidence of Na—K—Cl cotransport in airway smooth muscle. Am J Physiol, 1995. 268(4 Pt 1): p. L551-7.
Rubin A S, Cardoso P F: Bronchial thermoplasty in asthma. J. Bras. Pneumol. 2010; 36: 506-12
Stevens, E. L., et al., Furosemide differentially relaxes airway and vascular smooth muscle in fetal, newborn, and adult guinea pigs. Am Rev Respir Dis, 1992. 146(5 Pt 1): p. 1192-7.
Sunaga H, Zhang Y, Savarese J J, Emala C W: Gantacurium and CW002 do not potentiate muscarinic receptor-mediated airway smooth muscle constriction in guinea pigs. Anesthesiology 2010; 112: 892-9
Talwar D, Jindal S K: Effect of calcium channel antagonists on cholinergic bronchial responsiveness in asthma. J. Assoc. Physicians India 1993; 41: 279-80
Townsend, E. A., et al., Can we find better bronchodilators to relieve asthma symptoms?J Allergy (Cairo), 2012. 2012: p. 321949.
Verkman, A. S, and L. J. Galietta, Chloride channels as drug targets. Nat Rev Drug Discov, 2009. 8(2): p. 153-71.
Wafford K A, van Niel M B, Ma Q P, Horridge E, Herd M B, Peden D R, Belelli D, Lambert J J: Novel compounds selectively enhance delta subunit containing GABA A receptors and increase tonic currents in thalamus. Neuropharmacology 2009; 56: 182-9
Walker N M, Simpson J E, Levitt R C, Boyle K T, Clarke L L: Talniflumate increases survival in a cystic fibrosis mouse model of distal intestinal obstructive syndrome. Journal of Pharmacology and Experimental Therapeutics 2006; 317: 275-83
White T A, Xue A, Chini E N, Thompson M, Sieck G C, Wylam M E: Role of transient receptor potential C3 in TNF-alpha-enhanced calcium influx in human airway myocytes. American Journal of Respiratory Cell and Molecular Biology 2006; 35: 243-51
Woods, B. D. and R. N. Sladen, Perioperative considerations for the patient with asthma and bronchospasm. Br J Anaesth, 2009. 103 Suppl 1: p. i57-65.
Yamakage M, Chen X, Tsujiguchi N, Kamada Y, Namiki A: Different inhibitory effects of volatile anesthetics on T- and L-type voltage-dependent Ca²⁺ channels in porcine tracheal and bronchial smooth muscles. Anesthesiology 2001; 94: 683-93
Yim, P. D., et al., Novel expression of a functional glycine receptor chloride channel that attenuates contraction in airway smooth muscle. FASEB J, 2011. 25(5): p. 1706-17.
Zhang G H, Melvin J E: Membrane potential regulates Ca²⁺ uptake and inositol phosphate generation in rat sublingual mucous acini. Cell Calcium 1993; 14: 551-62
Zhang, C. H., et al., The transmembrane protein 16A Ca(2+)-activated Cl— channel in airway smooth muscle contributes to airway hyperresponsiveness. Am J Respir Crit. Care Med, 2013. 187(4): p. 374-81.

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

What is claimed is:

1. A method of treating or ameliorating the effects of a disease characterized by altered smooth muscle contractility comprising administering to a patient suffering from such a disease an effective amount of a calcium-activated chloride channel (CaCC) modulator and a sodium-potassium-chloride co-transporter (NKCC) modulator.

2. The method according to claim 1, wherein the CaCC modulator is a CaCC inhibitor.

3. The method according to claim 2, wherein the CaCC inhibitor is selected from the group consisting of niflumic acid, 5-nitro-2-(3-phenylpropylamino)-benzoate (NPPB), talnifumate, flufenamic acid, 4,4′-diisothiocyanatostilbene-2,2′-disulfonate (DIDS), indanyloxyacetic acid 94 (IAA-94), tamoxifen, 4-acetamido-4′-isothiocyanatostilbene-2,2′-disulfonic acid (SITS), anthracene-9-carboxylic acid (A9C), diphenylamine-2-carboxyl acid (DPC), 6-t-butyl-2-(furan-2-carboxamido)-4,5,6,7-tetrahydrobenzo[b]thiophene-3-carboxylic acid (CaCCᵢₙₕ-A01), 2-hydroxy-4-(4-p-tolylthiazol-2-ylaminobenzoic acid (CaCCᵢₙₕ-B01), morniflumate (Sanofi-Aventis, France), calcium-sensitive chloride channel antagonist (Takeda Pharmaceutical Co. Ltd., Japan), a pharmaceutically acceptable salt thereof, and combinations thereof.

4. The method according to claim 3, wherein the CaCC inhibitor is niflumic acid or a pharmaceutically acceptable salt thereof.

5. The method according to claim 3, wherein the CaCC inhibitor is

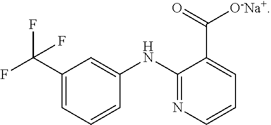

6. The method according to claim 1, wherein the NKCC modulator is a NKCC inhibitor.

7. The method according to claim 6, wherein the NKCC inhibitor is selected from the group consisting of bumetanide, furosemide, torasemide, azosemide, piretanide, tripamide, etozoline and its metabolite ozolinone, cicletanine, ethacrynic acid, muzolimine, LR-14-890 (Menarini, Italy), lemidosul (Sanofi-Aventis, France), M-12285 (Mochida, Japan), alilusem (Mochida, Japan), sulosemide sodium (Sano-Aventis, France), BTS-39542 (Abbott Laboratories, Abbott Park, Ill.), AY-31906 (Pfizer, New York, N.Y.), brocrinat (Sanofi-Aventis), SA-9000 (Santen, Japan), A-52773 (Abbott Laboratories), A-53385 (Abbott Laboratories), CL-301 (Chlorion Pharma, Canada), Abbott-49816 (Abbott Laboratories), ethacrynic acid (Telor Ophthalmic Pharmaceuticals, Wilmington, Mass.), a pharmaceutically acceptable salt thereof, and combinations thereof.

8. The method according to claim 7, wherein the NKCC inhibitor is selected from the group consisting of bumetanide, furosemide, a pharmaceutically acceptable salt thereof, and combinations thereof.

9. The method according to claim 8, wherein the NKCC inhibitor is bumetanide.

10. The method according to claim 1, wherein the disease is selected from the group consisting of asthma, chronic obstructive pulmonary disease (COPD), cystic fibrosis, adult respiratory distress syndrome, and bronchospasm.

11. The method according to claim 1, wherein the disease is asthma or COPD.

12. The method according to claim 1, wherein the CaCC modulator and the NKCC modulator are administered as part of a pharmaceutical composition.

13. The method according to claim 12, wherein the pharmaceutical composition is in a unit dosage form.

14. The method according to claim 12, wherein the pharmaceutical composition is co-administered with a β-agonist.

15. A pharmaceutical composition for treating or ameliorating the effects of a disease characterized by altered smooth muscle contractility, the composition comprising a pharmaceutically acceptable carrier, a CaCC modulator, and a NKCC modulator.

16. The pharmaceutical composition according to claim 15, wherein the disease is selected from the group consisting of asthma, chronic obstructive pulmonary disease (COPD), cystic fibrosis, adult respiratory distress syndrome, and bronchospasm.

17. The pharmaceutical composition according to claim 15, wherein the disease is asthma or COPD.

18. The pharmaceutical composition according to claim 15, which is in a unit dosage form.

19. The pharmaceutical composition according to claim 15, which is co-administered with a β-agonist.

20. A method of relaxing airway smooth muscle comprising administering to a patient in need thereof an effective amount of a CaCC modulator and a NKCC modulator.

21. The method according to claim 20, wherein the CaCC modulator is a CaCC inhibitor, and the NKCC modulator is a NKCC inhibitor.

22. A method of preventing the effects of a disease characterized by altered smooth muscle contractility comprising administering to a patient suffering from such a disease an effective amount of a calcium-activated chloride channel (CaCC) inhibitor and a sodium-potassium-chloride co-transporter (NKCC) inhibitor.

23. The method according to claim 22, wherein the CaCC inhibitor is selected from the group consisting of niflumic acid, 5-nitro-2-(3-phenylpropylamino)-benzoate (NPPB), talnifumate, flufenamic acid, 4,4′-diisothiocyanatostilbene-2,2′-disulfonate (DIDS), indanyloxyacetic acid 94 (IAA-94), tamoxifen, 4-acetamido-4′-isothiocyanatostilbene-2,2′-disulfonic acid (SITS), anthracene-9-carboxylic acid (A9C), diphenylamine-2-carboxyl acid (DPC), 6-t-butyl-2-(furan-2-carboxamido)-4,5,6,7-tetrahydrobenzo[b]thiophene-3-carboxylic acid (CaCCᵢₙₕ-A01), 2-hydroxy-4-(4-p-tolylthiazol-2-ylaminobenzoic acid (CaCCᵢₙₕ-B01), morniflumate (Sanofi-Aventis, France), calcium-sensitive chloride channel antagonist (Takeda Pharmaceutical Co. Ltd., Japan), 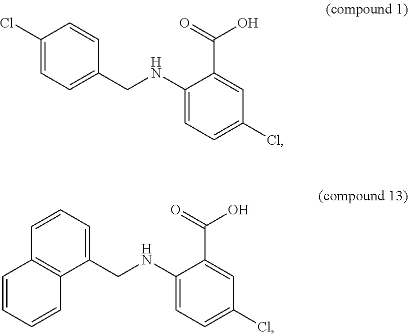 [SMILES: O=C(O)c1cc(Cl)ccc1NCc1ccc(Cl)cc1 ; O=C(O)c1cc(Cl)ccc1NCc1cccc2ccccc12]
a pharmaceutically acceptable salt thereof, and combinations thereof.

24. The method according to claim 23, wherein the CaCC inhibitor is niflumic acid or a pharmaceutically acceptable salt thereof.

25. The method according to claim 23, wherein the CaCC inhibitor is NPPB.

26. The method according to claim 23, wherein the CaCC inhibitor is

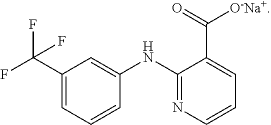

27. The method according to claim 23, wherein the CaCC inhibitor is

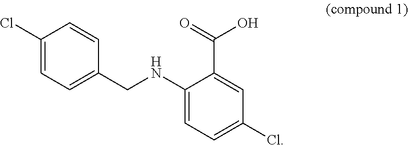 [SMILES: O=C(O)c1cc(Cl)ccc1NCc1ccc(Cl)cc1]

28. The method according to claim 23, wherein the CaCC inhibitor is

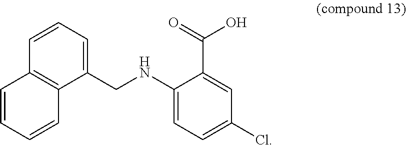 [SMILES: O=C(O)c1cc(Cl)ccc1NCc1cccc2ccccc12]

29. The method according to claim 22, wherein the NKCC inhibitor is selected from the group consisting of bumetanide, furosemide, torasemide, azosemide, piretanide, tripamide, etozoline and its metabolite ozolinone, cicletanine, ethacrynic acid, muzolimine, LR-14-890 (Menarini, Italy), lemidosul (Sanofi-Aventis, France), M-12285 (Mochida, Japan), alilusem (Mochida, Japan), sulosemide sodium (Sano-Aventis, France), BTS-39542 (Abbott Laboratories, Abbott Park, Ill.), AY-31906 (Pfizer, New York, N.Y.), brocrinat (Sanofi-Aventis), SA-9000 (Santen, Japan), A-52773 (Abbott Laboratories), A-53385 (Abbott Laboratories), CL-301 (Chlorion Pharma, Canada), Abbott-49816 (Abbott Laboratories), ethacrynic acid (Telor Ophthalmic Pharmaceuticals, Wilmington, Mass.), a pharmaceutically acceptable salt thereof, and combinations thereof.

30. The method according to claim 29, wherein the NKCC inhibitor is selected from the group consisting of bumetanide, furosemide, a pharmaceutically acceptable salt thereof, and combinations thereof.

31. The method according to claim 30, wherein the NKCC inhibitor is bumetanide.

32. The method according to claim 22, wherein the disease is selected from the group consisting of asthma, chronic obstructive pulmonary disease (COPD), cystic fibrosis, adult respiratory distress syndrome, and bronchospasm.

33. The method according to claim 22, wherein the disease is asthma or COPD.

34. The method according to claim 22, wherein the CaCC inhibitor and the NKCC inhibitor are administered as part of a pharmaceutical composition.

35. The method according to claim 32, wherein the pharmaceutical composition is in a unit dosage form.

36. The method according to claim 32, wherein the pharmaceutical composition is co-administered with a β-agonist.

37. A pharmaceutical composition for preventing the effects of a disease characterized by altered smooth muscle contractility, the composition comprising a pharmaceutically acceptable carrier, a CaCC inhibitor, and a NKCC inhibitor.

38. The pharmaceutical composition according to claim 37, wherein the disease is selected from the group consisting of asthma, chronic obstructive pulmonary disease (COPD), cystic fibrosis, adult respiratory distress syndrome, and bronchospasm.

39. The pharmaceutical composition according to claim 37, wherein the disease is asthma or COPD.

40. The pharmaceutical composition according to claim 37, which is in a unit dosage form.

41. The pharmaceutical composition according to claim 37, which is co-administered with a β-agonist.

42. A method of blocking the onset of an airway contraction comprising administering to a patient in need thereof an effective amount of a CaCC inhibitor and a NKCC inhibitor.

43. A pharmaceutical composition for treating or ameliorating the effects of a disease characterized by altered smooth muscle contractility comprising a pharmaceutically acceptable carrier and a CaCC inhibitor selected from the group consisting of 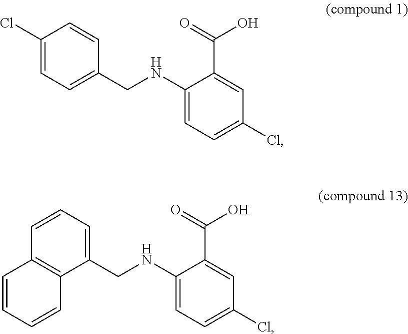 [SMILES: O=C(O)c1cc(Cl)ccc1NCc1ccc(Cl)cc1 ; O=C(O)c1cc(Cl)ccc1NCc1cccc2ccccc12]
a pharmaceutically acceptable salt thereof, and combinations thereof.

44. The pharmaceutical composition according to claim 43, wherein the disease is asthma or COPD.

45. The pharmaceutical composition according to claim 43, wherein the CaCC inhibitor is 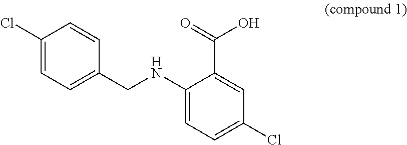 [SMILES: O=C(O)c1cc(Cl)ccc1NCc1ccc(Cl)cc1]
or a pharmaceutically acceptable salt thereof.

46. The pharmaceutical composition according to claim 43, wherein the CaCC inhibitor is 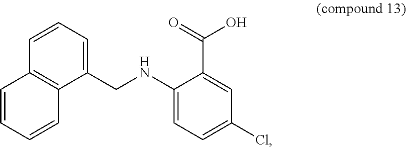 [SMILES: O=C(O)c1cc(Cl)ccc1NCc1cccc2ccccc12]
or a pharmaceutically acceptable salt thereof.

47. A method for treating or ameliorating the effects of a disease characterized by altered smooth muscle contractility comprising administering to a patient suffering from such a disease an effective amount of a CaCC inhibitor selected from the group consisting of 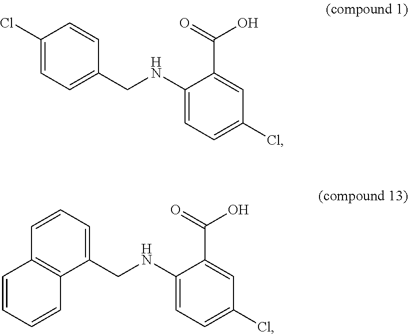 [SMILES: O=C(O)c1cc(Cl)ccc1NCc1ccc(Cl)cc1 ; O=C(O)c1cc(Cl)ccc1NCc1cccc2ccccc12]
a pharmaceutically acceptable salt thereof, and combinations thereof.

48. The method according to claim 47, wherein the disease is asthma or COPD.

49. The method according to claim 47, wherein the CaCC inhibitor is 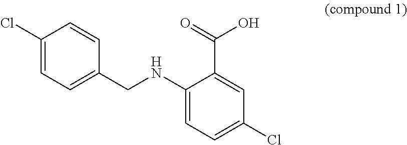 [SMILES: O=C(O)c1cc(Cl)ccc1NCc1ccc(Cl)cc1]
or a pharmaceutically acceptable salt thereof.

50. The method according to claim 47, wherein the CaCC inhibitor is 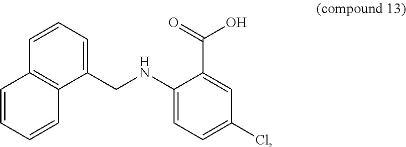 [SMILES: O=C(O)c1cc(Cl)ccc1NCc1cccc2ccccc12]
or a pharmaceutically acceptable salt thereof.

51. A method of treating or ameliorating the effects of a disease characterized by altered smooth muscle contractility comprising administering to a patient suffering from such a disease an effective amount of a sodium-potassium-chloride co-transporter (NKCC) inhibitor and a calcium-activated chloride channel (CaCC) inhibitor selected from the group consisting of 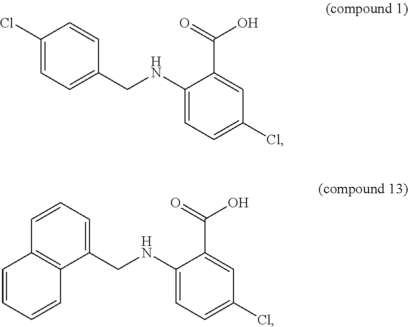 [SMILES: O=C(O)c1cc(Cl)ccc1NCc1ccc(Cl)cc1 ; O=C(O)c1cc(Cl)ccc1NCc1cccc2ccccc12]
a pharmaceutically acceptable salt thereof, and combinations thereof.

52. A method of treating or ameliorating the effects of a disease characterized by altered smooth muscle contractility comprising administering to a patient suffering from such a disease an effective amount of a CaCC modulator, which is: 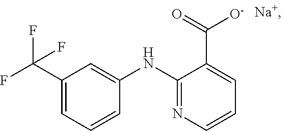 [SMILES: O=C([O-])c1cccnc1Nc1cccc(C(F)(F)F)c1]
and a NKCC modulator.

53. The method according to claim 52, wherein the NKCC modulator is a NKCC inhibitor.

54. The method according to claim 53, wherein the NKCC inhibitor is selected from the group consisting of bumetanide, furosemide, torasemide, azosemide, piretanide, tripamide, etozoline and its metabolite ozolinone, cicletanine, ethacrynic acid, muzolimine, LR-14-890 (Menarini, Italy), lemidosul (Sanofi-Aventis, France), M-12285 (Mochida, Japan), alilusem (Mochida, Japan), sulosemide sodium (Sano-Aventis, France), BTS-39542 (Abbott Laboratories, Abbott Park, Ill.), AY-31906 (Pfizer, New York, N.Y.), brocrinat (Sanofi-Aventis), SA-9000 (Santen, Japan), A-52773 (Abbott Laboratories), A-53385 (Abbott Laboratories), CL-301 (Chlorion Pharma, Canada), Abbott-49816 (Abbott Laboratories), ethacrynic acid (Telor Ophthalmic Pharmaceuticals, Wilmington, Mass.), a pharmaceutically acceptable salt thereof, and combinations thereof.

55. The method according to claim 52, wherein the disease is selected from the group consisting of asthma, chronic obstructive pulmonary disease (COPD), cystic fibrosis, adult respiratory distress syndrome, and bronchospasm.

56. The method according to claim 52, wherein the disease is asthma or COPD.

57. The method according to claim 52, wherein the CaCC modulator and the NKCC modulator are administered as part of a pharmaceutical composition.

58. The method according to claim 57, wherein the pharmaceutical composition is in a unit dosage form.

59. The method according to claim 57, wherein the pharmaceutical composition is co-administered with a β-agonist.

60. A pharmaceutical composition for treating or ameliorating the effects of a disease characterized by altered smooth muscle contractility, the composition comprising a pharmaceutically acceptable carrier, a CaCC modulator, which is 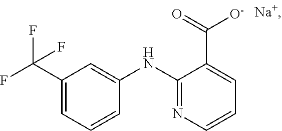 [SMILES: O=C([O-])c1cccnc1Nc1cccc(C(F)(F)F)c1]
and a NKCC modulator.

61. The pharmaceutical composition according to claim 60, wherein the NKCC modulator is a NKCC inhibitor.

62. The pharmaceutical composition according to claim 61, wherein the NKCC inhibitor is selected from the group consisting of bumetanide, furosemide, torasemide, azosemide, piretanide, tripamide, etozoline and its metabolite ozolinone, cicletanine, ethacrynic acid, muzolimine, LR-14-890 (Menarini, Italy), lemidosul (Sanofi-Aventis, France), M-12285 (Mochida, Japan), alilusem (Mochida, Japan), sulosemide sodium (Sano-Aventis, France), BTS-39542 (Abbott Laboratories, Abbott Park, Ill.), AY-31906 (Pfizer, New York, N.Y.), brocrinat (Sanofi-Aventis), SA-9000 (Santen, Japan), A-52773 (Abbott Laboratories), A-53385 (Abbott Laboratories), CL-301 (Chlorion Pharma, Canada), Abbott-49816 (Abbott Laboratories), ethacrynic acid (Telor Ophthalmic Pharmaceuticals, Wilmington, Mass.), a pharmaceutically acceptable salt thereof, and combinations thereof.

63. The pharmaceutical composition according to claim 60, wherein the disease is selected from the group consisting of asthma, chronic obstructive pulmonary disease (COPD), cystic fibrosis, adult respiratory distress syndrome, and bronchospasm.

64. The pharmaceutical composition according to claim 60, wherein the disease is asthma or COPD.

65. The pharmaceutical composition according to claim 60, which is in a unit dosage form.

66. The pharmaceutical composition according to claim 60, which is co-administered with a β-agonist.
